SEQUENCE LISTING (199 sequences)

SEQ ID NO: 1 - protein, 34 aa, Momordica cochinchinensis
CGSGSDGGVCPKILQRCRRDSDCPGACICRGNGY

SEQ ID NO: 2 - protein, 34 aa, Artificial Sequence
  source: /note=“Description of Artificial Sequence Synthetic polypeptide”
CGSGSDGGVCAKILQRCRRDSDCPGACICRGNGY

SEQ ID NO: 3 - protein, 34 aa, Artificial Sequence
  source: /note=“Description of Artificial Sequence Synthetic polypeptide”
CGSGSDGGVCPAILQRCRRDSDCPGACICRGNGY

SEQ ID NO: 4 - protein, 34 aa, Artificial Sequence
  source: /note=“Description of Artificial Sequence Synthetic polypeptide”
CGSGSDGGVCPKTLQRCRRDSDCPGACICRGNGY

SEQ ID NO: 5 - protein, 34 aa, Artificial Sequence
  source: /note=“Description of Artificial Sequence Synthetic polypeptide”
CGSGSDGGVCPKIAQRCRRDSDCPGACICRGNGY

SEQ ID NO: 6 - protein, 34 aa, Artificial Sequence
  source: /note=“Description of Artificial Sequence Synthetic polypeptide”
CGSGSDGGVCPKILGRCRRDSDCPGACICRGNGY

SEQ ID NO: 7 - protein, 34 aa, Artificial Sequence
  source: /note=“Description of Artificial Sequence Synthetic polypeptide”
CGSGSDGGVCPKILMRCRRDSDCPGACICRGNGY

SEQ ID NO: 8 - protein, 34 aa, Artificial Sequence
  source: /note=“Description of Artificial Sequence Synthetic polypeptide”
CGSGSDGGVCPKILQACRRDSDCPGACICRGNGY

SEQ ID NO: 9 - protein, 34 aa, Artificial Sequence
  source: /note=“Description of Artificial Sequence Synthetic polypeptide”
CGSGSDGGVCPKILQRCRRASDCPGACICRGNGY

SEQ ID NO: 10 - protein, 34 aa, Artificial Sequence
  source: /note=“Description of Artificial Sequence Synthetic polypeptide”
CGSGSDGGVCPKILQRCRRDADCPGACICRGNGY

SEQ ID NO: 11 - protein, 34 aa, Artificial Sequence
  source: /note=“Description of Artificial Sequence Synthetic polypeptide”
CGSGSDGGVCPKILQRCRRDSDCPAACICRGNGY

SEQ ID NO: 12 - protein, 34 aa, Artificial Sequence
  source: /note=“Description of Artificial Sequence Synthetic polypeptide”
CGSGSDGGVCPKILQRCRRDSDCPGGCICRGNGY

SEQ ID NO: 13 - protein, 34 aa, Artificial Sequence
  source: /note=“Description of Artificial Sequence Synthetic polypeptide”
CGSGSDGGVCPKILQRCRRDSDCPGACICRGNGA

SEQ ID NO: 14 - protein, 34 aa, Artificial Sequence
  source: /note=“Description of Artificial Sequence Synthetic polypeptide”
CGSGSDGGACPKILQRCRRDSDCPGACICRGNGY

SEQ ID NO: 15 - protein, 34 aa, Artificial Sequence
  source: /note=“Description of Artificial Sequence Synthetic polypeptide”
CGSGSDGGSCPKILQRCRRDSDCPGACICRGNGY

SEQ ID NO: 16 - protein, 34 aa, Artificial Sequence
  source: /note=“Description of Artificial Sequence Synthetic polypeptide”
CGSGSDGGVCPKILQRCARDSDCPGACICRGNGY

SEQ ID NO: 17 - protein, 34 aa, Artificial Sequence
  source: /note=“Description of Artificial Sequence Synthetic polypeptide”
CGSGSDGGVCPKILQRCGRDSDCPGACICRGNGY

SEQ ID NO: 18 - protein, 34 aa, Artificial Sequence
  source: /note=“Description of Artificial Sequence Synthetic polypeptide”
CGSGSDGGVCPKILQRCRVDSDCPGACICRGNGY

SEQ ID NO: 19 - protein, 34 aa, Artificial Sequence
  source: /note=“Description of Artificial Sequence Synthetic polypeptide”
CGSGSDGGVCPKILQRCRRDSACPGACICRGNGY

SEQ ID NO: 20 - protein, 34 aa, Artificial Sequence
  source: /note=“Description of Artificial Sequence Synthetic polypeptide”
CGSGSDGGVCPKILQRCRRDSDCAGACICRGNGY

SEQ ID NO: 21 - protein, 34 aa, Artificial Sequence
  source: /note=“Description of Artificial Sequence Synthetic polypeptide”
CGSGSDGGVCPKILQRCRRDSDCPGACGCRGNGY

SEQ ID NO: 22 - protein, 34 aa, Artificial Sequence
  source: /note=“Description of Artificial Sequence Synthetic polypeptide”
CGSGSDGGVCPKILQRCRRDSDCPGACICWGNGY

SEQ ID NO: 23 - protein, 34 aa, Artificial Sequence
  source: /note=“Description of Artificial Sequence Synthetic polypeptide”
CGSGSDGGVCPKILQRCRRDSDCPGACICRANGY

SEQ ID NO: 24 - protein, 34 aa, Artificial Sequence
  source: /note=“Description of Artificial Sequence Synthetic polypeptide”
CGSGSDGGVCPKILQRCRRDSDCPGACICRGWGY

SEQ ID NO: 25 - protein, 34 aa, Artificial Sequence
  source: /note=“Description of Artificial Sequence Synthetic polypeptide”
CGSGSDGGVCPKILQRCRRDSDCPGACICRGNPY

SEQ ID NO: 26 - protein, 34 aa, Artificial Sequence
  source: /note=“Description of Artificial Sequence Synthetic polypeptide”
CGSGSDGGVCPKILQRCRRDSDCPGACICRGNGW

SEQ ID NO: 27 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtggtaacggttac

SEQ ID NO: 28 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcaggattttcgggcaaacaccaccgtcagaaccagaaccgcaca

SEQ ID NO: 29 - DNA, 106 bp, Artificial Sequence
  source: /note=“Description of Artificial Sequence Synthetic polynucleotide”
tatgtgcggttctggttctgacggtggtgcttgcccgaaaatcctgcagcgttgccgtcgtgactctgactgcccgggtgcttgcatctgccgtggtaacggttac

SEQ ID NO: 30 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcaggattttcgggcaagcaccaccgtcagaaccagaaccgcaca

SEQ ID NO: 31 - DNA, 106 bp, Artificial Sequence
  source: /note=“Description of Artificial Sequence Synthetic polynucleotide”
tatgtgcggttctggttctgacggtggttcttgcccgaaaatcctgcagcgttgccgtcgtgactctgactgcccgggtgcttgcatctgccgtggtaacggttac

SEQ ID NO: 32 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcaggattttcgggcaagaaccaccgtcagaaccagaaccgcaca

SEQ ID NO: 33 - DNA, 106 bp, Artificial Sequence
  source: /note=“Description of Artificial Sequence Synthetic polynucleotide”
tatgtgcggttctggttctgacggtggtgtttgcgctaaaatcctgcagcgttgccgtcgtgactctgactgcccgggtgcttgcatctgccgtggtaacggttac

SEQ ID NO: 34 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcaggattttagcgcaaacaccaccgtcagaaccagaaccgcaca

SEQ ID NO: 35 - DNA, 106 bp, Artificial Sequence
  source: /note=“Description of Artificial Sequence Synthetic polynucleotide”
tatgtgcggttctggttctgacggtggtgtttgcccggctatcctgcagcgttgccgtcgtgactctgactgcccgggtgcttgcatctgccgtggtaacggttac

SEQ ID NO: 36 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcaggatagccgggcaaacaccaccgtcagaaccagaaccgcaca

SEQ ID NO: 37 - DNA, 106 bp, Artificial Sequence
  source: /note=“Description of Artificial Sequence Synthetic polynucleotide”
tatgtgcggttctggttctgacggtggtgtttgcccgaaaaccctgcagcgttgccgtcgtgactctgactgcccgggtgcttgcatctgccgtggtaacggttac

SEQ ID NO: 38 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgcagggttttcgggcaaacaccaccgtcagaaccagaaccgcaca

SEQ ID NO: 39 - DNA, 106 bp, Artificial Sequence
  source: /note=“Description of Artificial Sequence Synthetic polynucleotide”
tatgtgcggttctggttctgacggtggtgtttgcccgaaaatcgctcagcgttgccgtcgtgactctgactgcccgggtgcttgcatctgccgtggtaacggttac

SEQ ID NO: 40 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgctgagcgattttcgggcaaacaccaccgtcagaaccagaaccgcaca

SEQ ID NO: 41 - DNA, 106 bp, Artificial Sequence
  source: /note=“Description of Artificial Sequence Synthetic polynucleotide”
tatgtgcggttctggttctgacggtggtgtttgcccgaaaatcctgggtcgttgccgtcgtgactctgactgcccgggtgcttgcatctgccgtggtaacggttac

SEQ ID NO: 42 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgacccaggattttcgggcaaacaccaccgtcagaaccagaaccgcaca

SEQ ID NO: 43 - DNA, 106 bp, Artificial Sequence
  source: /note=“Description of Artificial Sequence Synthetic polynucleotide”
tatgtgcggttctggttctgacggtggtgtttgcccgaaaatcctgatgcgttgccgtcgtgactctgactgcccgggtgcttgcatctgccgtggtaacggttac

SEQ ID NO: 44 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaacgcatcaggattttcgggcaaacaccaccgtcagaaccagaaccgcaca

SEQ ID NO: 45 - DNA, 106 bp, Artificial Sequence
  source: /note=“Description of Artificial Sequence Synthetic polynucleotide”
tatgtgcggttctggttctgacggtggtgtttgcccgaaaatcctgcaggcttgccgtcgtgactctgactgcccgggtgcttgcatctgccgtggtaacggttac

SEQ ID NO: 46 - DNA, 107 bp, Artificial Sequence
  source: /note=“Description of Artificial Sequence Synthetic polynucleotide”
gcagtaaccgttaccacggcagatgcaagcacccgggcagtcagagtcacgacggcaagcctgcaggattttcgggcaaacaccaccgtcagaaccagaaccgcaca

SEQ ID NO: 47 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgcgctcgtgactctgactgcccgggtgcttgcatctgccgtggtaacggttac

SEQ ID NO: 48 - DNA, 107 bp, Artificial Sequence
  source: /note=“Description of Artificial Sequence Synthetic polynucleotide”
gcagtaaccgttaccacggcagatgcaagcacccgggcagtcagagtcacgagcgcaacgctgcaggattttcgggcaaacaccaccgtcagaaccagaaccgcaca

SEQ ID NO: 49 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgcggtcgtgactctgactgcccgggtgcttgcatctgccgtggtaacggttac

SEQ ID NO: 50 - DNA, 107 bp, Artificial Sequence
  source: /note=“Description of Artificial Sequence Synthetic polynucleotide”
gcagtaaccgttaccacggcagatgcaagcacccgggcagtcagagtcacgaccgcaacgctgcaggattttcgggcaaacaccaccgtcagaaccagaaccgcaca

SEQ ID NO: 51 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtgttgactctgactgcccgggtgcttgcatctgccgtggtaacggttac

SEQ ID NO: 52 - DNA, 107 bp, Artificial Sequence
  source: /note=“Description of Artificial Sequence Synthetic polynucleotide”
gcagtaaccgttaccacggcagatgcaagcacccgggcagtcagagtcaacacggcaacgctgcaggattttcgggcaaacaccaccgtcagaaccagaaccgcaca

SEQ ID NO: 53 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgcttctgactgcccgggtgcttgcatctgccgtggtaacggttac

SEQ ID NO: 54 - DNA, 107 bp, Artificial Sequence
  source: /note=“Description of Artificial Sequence Synthetic polynucleotide”
gcagtaaccgttaccacggcagatgcaagcacccgggcagtcagaagcacgacggcaacgctgcaggattttcgggcaaacaccaccgtcagaaccagaaccgcaca

SEQ ID NO: 55 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgacgctgactgcccgggtgcttgcatctgccgtggtaacggttac

SEQ ID NO: 56 - DNA, 107 bp, Artificial Sequence
  source: /note=“Description of Artificial Sequence Synthetic polynucleotide”
gcagtaaccgttaccacggcagatgcaagcacccgggcagtcagcgtcacgacggcaacgctgcaggattttcgggcaaacaccaccgtcagaaccagaaccgcaca

SEQ ID NO: 57 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgcttgcccgggtgcttgcatctgccgtggtaacggttac

SEQ ID NO: 58 - DNA, 107 bp, Artificial Sequence
  source: /note=“Description of Artificial Sequence Synthetic polynucleotide”
gcagtaaccgttaccacggcagatgcaagcacccgggcaagcagagtcacgacggcaacgctgcaggattttcgggcaaacaccaccgtcagaaccagaaccgcaca

SEQ ID NO: 59 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcgctggtgcttgcatctgccgtggtaacggttac

SEQ ID NO: 60 - DNA, 107 bp, Artificial Sequence
  source: /note=“Description of Artificial Sequence Synthetic polynucleotide”
gcagtaaccgttaccacggcagatgcaagcaccagcgcagtcagagtcacgacggcaacgctgcaggattttcgggcaaacaccaccgtcagaaccagaaccgcaca

SEQ ID NO: 61 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccggctgcttgcatctgccgtggtaacggttac

SEQ ID NO: 62 - DNA, 107 bp, Artificial Sequence
  source: /note=“Description of Artificial Sequence Synthetic polynucleotide”
gcagtaaccgttaccacggcagatgcaagcagccgggcagtcagagtcacgacggcaacgctgcaggattttcgggcaaacaccaccgtcagaaccagaaccgcaca

SEQ ID NO: 63 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtggttgcatctgccgtggtaacggttac

SEQ ID NO: 64 - DNA, 107 bp, Artificial Sequence
  source: /note=“Description of Artificial Sequence Synthetic polynucleotide”
gcagtaaccgttaccacggcagatgcaaccacccgggcagtcagagtcacgacggcaacgctgcaggattttcgggcaaacaccaccgtcagaaccagaaccgcaca

SEQ ID NO: 65 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcggttgccgtggtaacggttac

SEQ ID NO: 66 - DNA, 107 bp, Artificial Sequence
  source: /note=“Description of Artificial Sequence Synthetic polynucleotide”
gcagtaaccgttaccacggcaaccgcaagcacccgggcagtcagagtcacgacggcaacgctgcaggattttcgggcaaacaccaccgtcagaaccagaaccgcaca

SEQ ID NO: 67 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgctggggtaacggttac

SEQ ID NO: 68 - DNA, 107 bp, Artificial Sequence
  source: /note=“Description of Artificial Sequence Synthetic polynucleotide”
gcagtaaccgttaccccagcagatgcaagcacccgggcagtcagagtcacgacggcaacgctgcaggattttcgggcaaacaccaccgtcagaaccagaaccgcaca

SEQ ID NO: 69 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtgctaacggttac

SEQ ID NO: 70 - DNA, 107 bp, Artificial Sequence
  source: /note=“Description of Artificial Sequence Synthetic polynucleotide”
gcagtaaccgttagcacggcagatgcaagcacccgggcagtcagagtcacgacggcaacgctgcaggattttcgggcaaacaccaccgtcagaaccagaaccgcaca

SEQ ID NO: 71 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtggttggggttac

SEQ ID NO: 72 - DNA, 107 bp, Artificial Sequence
  source: /note=“Description of Artificial Sequence Synthetic polynucleotide”
gcagtaaccccaaccacggcagatgcaagcacccgggcagtcagagtcacgacggcaacgctgcaggattttcgggcaaacaccaccgtcagaaccagaaccgcaca

SEQ ID NO: 73 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtggtaacccgtac

SEQ ID NO: 74 - DNA, 107 bp, Artificial Sequence
  source: /note=“Description of Artificial Sequence Synthetic polynucleotide”
gcagtacgggttaccacggcagatgcaagcacccgggcagtcagagtcacgacggcaacgctgcaggattttcgggcaaacaccaccgtcagaaccagaaccgcaca

SEQ ID NO: 75 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtggtaacggttgg

SEQ ID NO: 76 - DNA, 107 bp, Artificial Sequence
  source: /note=“Description of Artificial Sequence Synthetic polynucleotide”
gcaccaaccgttaccacggcagatgcaagcacccgggcagtcagagtcacgacggcaacgctgcaggattttcgggcaaacaccaccgtcagaaccagaaccgcaca

SEQ ID NO: 77 - DNA, 106 bp, Artificial Sequence
  source: /note=“Description of Artificial Sequence Synthetic polynucleotide”
tatgtgcggttctggttctgacggtggtgtttgcccgaaaatcctgcagcgttgccgtcgtgactctgactgcccgggtgcttgcatctgccgtggtaacggtgct

SEQ ID NO: 78 - DNA, 107 bp, Artificial Sequence
  source: /note=“Description of Artificial Sequence Synthetic polynucleotide”
gcaagcaccgttaccacggcagatgcaagcacccgggcagtcagagtcacgacggcaacgctgcaggattttcgggcaaacaccaccgtcagaaccagaaccgcaca

SEQ ID NO: 79 - protein, 34 aa, Artificial Sequence
  source: /note=“Description of Artificial Sequence Synthetic polypeptide”
VCPKILQRCRRDSDCPGACICRGNGYCGSGSDGG

SEQ ID NO: 80 - protein, 34 aa, Artificial Sequence
  source: /note=“Description of Artificial Sequence Synthetic polypeptide”
VCPAILQRCRRDSDCPGACICRGNGYCGSGSDGG

SEQ ID NO: 81 - protein, 15 aa, Artificial Sequence
  source: /note=“Description of Artificial Sequence Synthetic consensus peptide”
RRKKVYPYPMEGTIA

SEQ ID NO: 82 - protein, 18 aa, Artificial Sequence
  source: /note=“Description of Artificial Sequence Synthetic peptide”
GGSGGSGGSGGSGGSGGS

SEQ ID NO: 83 - DNA, 29 bp, Artificial Sequence
  source: /note=“Description of Artificial Sequence Synthetic primer”
ggccaggagtgctagcatgtctaaaggtg

SEQ ID NO: 84 - DNA, 32 bp, Artificial Sequence
  source: /note=“Description of Artificial Sequence Synthetic primer”
ggtggtggtgggatcctttgtacaattcatcc

SEQ ID NO: 85 - DNA, 33 bp, Artificial Sequence
  source: /note=“Description of Artificial Sequence Synthetic primer”
cactaaggccaggaaagcttcgatgtctaaagg

SEQ ID NO: 86 - DNA, 33 bp, Artificial Sequence
  source: /note=“Description of Artificial Sequence Synthetic primer”
ccttagtggtggtgctcgagttatttgtacaat

SEQ ID NO: 87 - DNA, 33 bp, Artificial Sequence
  source: /note=“Description of Artificial Sequence Synthetic primer”
atatatgagctctagcatgtctaaaggtgaaga

SEQ ID NO: 88 - DNA, 33 bp, Artificial Sequence
  source: /note=“Description of Artificial Sequence Synthetic primer”
aatataggtaccttgtacaattcattcataccc

SEQ ID NO: 89 - DNA, 27 bp, Artificial Sequence
  source: /note=“Description of Artificial Sequence Synthetic primer”
taaggatccggcgggcggtcatggtga

SEQ ID NO: 90 - DNA, 21 bp, Artificial Sequence
  source: /note=“Description of Artificial Sequence Synthetic primer”
gcatctcccgtgatgcaggaa

SEQ ID NO: 91 - DNA, 52 bp, Artificial Sequence
  source: /note=“Description of Artificial Sequence Synthetic oligonucleotide”
gatcccgtcgtaaaaaagtttatccgtatccgatggaaggtaccatcgccca

SEQ ID NO: 92 - DNA, 52 bp, Artificial Sequence
  source: /note=“Description of Artificial Sequence Synthetic oligonucleotide”
agcttgggcgatggtaccttccatcggatacggataaacttttttacgacgg

SEQ ID NO: 93 - DNA, 70 bp, Artificial Sequence
  source: /note=“Description of Artificial Sequence Synthetic oligonucleotide”
gatccggtggcagccgtcgtaaaaaagtttatccgtatccgatggaaccgaccatcgccggtggcagcca

SEQ ID NO: 94 - DNA, 70 bp, Artificial Sequence
  source: /note=“Description of Artificial Sequence Synthetic oligonucleotide”
agcttggctgccaccggcgatggtcggttccatcggatacggataaacttttttacgacggctgccaccg

SEQ ID NO: 95 - DNA, 88 bp, Artificial Sequence
  source: /note=“Description of Artificial Sequence Synthetic oligonucleotide”
gatccggtggcagcggtggcagccgtcgtaaaaaagtttatccgtatccgatggaaccgaccatcgccggtggcagcggtggcagcca

SEQ ID NO: 96 - DNA, 88 bp, Artificial Sequence
  source: /note=“Description of Artificial Sequence Synthetic oligonucleotide”
agcttggctgccaccgctgccaccggcgatggtcggttccatcggatacggataaacttttttacgacggctgccaccgctgccaccg

SEQ ID NO: 97 - DNA, 61 bp, Artificial Sequence
  source: /note=“Description of Artificial Sequence Synthetic oligonucleotide”
gatccggtggcagcggtggcagcggtggcagcggtggcagccgtcgtaaaaaagtttatcc

SEQ ID NO: 98 - DNA, 62 bp, Artificial Sequence
  source: /note=“Description of Artificial Sequence Synthetic oligonucleotide”
gatacggataaacttttttacgacggctgccaccgctgccaccgctgccaccgctgccaccg

SEQ ID NO: 99 - DNA, 63 bp, Artificial Sequence
  source: /note=“Description of Artificial Sequence Synthetic oligonucleotide”
gtatccgatggaaccgaccatcgccggtggcagcggtggcagcggtggcagcggtggcagcca

SEQ ID NO: 100 - DNA, 62 bp, Artificial Sequence
  source: /note=“Description of Artificial Sequence Synthetic oligonucleotide”
agcttggctgccaccgctgccaccgctgccaccgctgccaccggcgatggtcggttccatcg

SEQ ID NO: 101 - DNA, 79 bp, Artificial Sequence
  source: /note=“Description of Artificial Sequence Synthetic oligonucleotide”
gatccggtggcagcggtggcagcggtggcagcggtggcagcggtggcagcggtggcagccgtcgtaaaaaagtttatcc

SEQ ID NO: 102 - DNA, 80 bp, Artificial Sequence
  source: /note=“Description of Artificial Sequence Synthetic oligonucleotide”
gatacggataaacttttttacgacggctgccaccgctgccaccgctgccaccgctgccaccgctgccaccgctgccaccg

SEQ ID NO: 103 - DNA, 81 bp, Artificial Sequence
  source: /note=“Description of Artificial Sequence Synthetic oligonucleotide”
gtatccgatggaaccgaccatcgccggtggcagcggtggcagcggtggcagcggtggcagcggtggcagcggtggcagcca

SEQ ID NO: 104 - DNA, 80 bp, Artificial Sequence
  source: /note=“Description of Artificial Sequence Synthetic oligonucleotide”
agcttggctgccaccgctgccaccgctgccaccgctgccaccgctgccaccgctgccaccggcgatggtcggttccatcg

SEQ ID NO: 105 - protein, 29 aa, Oldenlandia affinis
VCGETCVGGTCNTPGCTCSWPVCTRNGLP

SEQ ID NO: 106 - protein, 29 aa, Oldenlandia affinis
VCGETCFGGTCNTPGCSCTWPICTRDGLP

SEQ ID NO: 107 - protein, 30 aa, Oldenlandia affinis
TCGETCFGGTCNTPGCTCDPWPICTRDGLP

SEQ ID NO: 108 - protein, 34 aa, Momordica cochinchinensis; identical to SEQ ID NO: 79

SEQ ID NO: 109 - protein, 34 aa, Momordica cochinchinensis
VCPKILKKCRRDSDCPGACICRGNGYCGSGSDGG

SEQ ID NO: 110 - protein, 33 aa, Momordica cochinchinensis
VCPKILQRCRRDSDCPGACICRGNGYCGSGSDG

SEQ ID NO: 111 - protein, 30 aa, Momordica charantia
ERRCPRILKQCKRDSDCPGECICMAHGFCG

SEQ ID NO: 112 - protein, 30 aa, Momordica charantia
ERGCPRILKQCKQDSDCPGECICMAHGFCG

SEQ ID NO: 113 - protein, 29 aa, Bryonia dioica
RGCPRILMRCKRDSDCLAGCVCQKNGYCG

SEQ ID NO: 114 - protein, 32 aa, Cucurbita pepo
HEERVCPKILMECKKDSDCLAECICLEHGYCG

SEQ ID NO: 115 - protein, 32 aa, Cucurbita maxima
HEERVCPRILMKCKKDSDCLAECVCLEHGYCG

SEQ ID NO: 116 - protein, 29 aa, Ecballium elaterium
GCPRILMRCKQDSDCLAGCVCGPNGFCGS

SEQ ID NO: 117 - protein, 30 aa, Cucumis sativus
MMCPRILMKCKHDSDCLPGCVCLEHIEYCG

SEQ ID NO: 118 - protein, 31 aa, Cucumis sativus
MVCPKILMKCKHDSDCLLDCVCLEDIGYCGV

SEQ ID NO: 119 - protein, 29 aa, Momordica repens
CICPRILMECKRDSDCLAQCVCKRQCYCC

SEQ ID NO: 120 - protein, 28 aa, Luffa cyclindrica
ICPRILMPCSSDSDCLAECICLENGFCG

SEQ ID NO: 121 - protein, 29 aa, Cucurbita maxima
RVCPRILMECKKDSDCLAECVCLEHGYCG

SEQ ID NO: 122 - protein, 30 aa, Cucumis melo
QRMCPKILMKCKQDSDCLLDCVCLKEGFCG

SEQ ID NO: 123 - protein, 28 aa, Cucumis melo
MCPKILMKCKQDSDCLLDCVCLKEGFCG

SEQ ID NO: 124 - protein, 29 aa, Luffa cyclindrica
RICPRILMECSSDSDCLAECICLENGFCG

SEQ ID NO: 125 - protein, 30 aa, Luffa cyclindrica
RICPRILMECSSDSDCLAECICLEQDGFCG

SEQ ID NO: 126 - protein, 29 aa, Luffa cyclindrica
RICPRILMECSSDSDCLAECICLEQGFCG

SEQ ID NO: 127 - protein, 28 aa, Momordica charantia
RICPRIWMECKRDSDCMAQCICVDGHCG

SEQ ID NO: 128 - protein, 86 aa, Bacillus amyloliquefaciens
ITKSEAQALGWVASKGNLADVAPGKSIGGDIFSNREGKLPGKSGRTWREADINYTSGFRNSDRILYSSDWLIYKTTDHYQTFTKIR

SEQ ID NO: 129 - protein, 86 aa, Bacillus amyloliquefaciens
ITKSEAQALGWVASKGNLADVAPGKSIGGDIFSNREGKLPAKSGRTWREADINYTSGFRNSDRILYSSDWLIYKTTDHYKTFTKIR

SEQ ID NO: 130 - protein, 86 aa, Bacillus amyloliquefaciens
ITKSQASALGWVASKGNLAEVAPGKSIGGDVFSNREGRLPSASGRTWREADINYVSGFRNADRLVYSSDWLIYKTTDHYATFTRIR

SEQ ID NO: 131 - protein, 86 aa, Bacillus amyloliquefaciens; identical to SEQ ID NO: 130

SEQ ID NO: 132 - protein, 29 aa, Oldenlandia affinis
CGETCVGGTCNTPGCTCSWPVCTRNGLPV

SEQ ID NO: 133 - protein, 30 aa, Oldenlandia affinis
MCGETCVGGTCNTPGCTCSWPVCTRNGLPV

SEQ ID NO: 134 - protein, 30 aa, Oldenlandia affinis
MCVGGTCNTPGCTCSWPVCTRNGLPVCGET

SEQ ID NO: 135 - protein, 30 aa, Oldenlandia affinis
MCNTPGCTCSWPVCTRNGLPVCGETCVGGT

SEQ ID NO: 136 - protein, 30 aa, Oldenlandia affinis
MCTCSWPVCTRNGLPVCGETCVGGTCNTPG

SEQ ID NO: 137 - protein, 30 aa, Oldenlandia affinis
MCSWPVCTRNGLPVCGETCVGGTCNTPGCT

SEQ ID NO: 138 - protein, 30 aa, Oldenlandia affinis
MCTRNGLPVCGETCVGGTCNTPGCTCSWPV

SEQ ID NO: 139 - protein, 34 aa, Momordica cochinchinensis; identical to SEQ ID NO: 79

SEQ ID NO: 140 - protein, 51 aa, Artificial Sequence
  source: /note=“Description of Artificial Sequence Synthetic polypeptide”
GGSGGSGGSGGSGGSGGSRRKKVYPYPMEGTIAGGSGGSGGSGGSGGSGGS

SEQ ID NO: 141 - protein, 18 aa, Artificial Sequence
  source: /note=“Description of Artificial Sequence Synthetic peptide”
GGSGGSGGSGGSGGSGGS

SEQ ID NO: 142 - DNA, 143 bp, Momordica cochinchinensis
  CDS (1)..(143)
  modified_base (67)..(68): a, c, t, g, unknown or other
aaagaattccatatgtgcggttctggttctgacggtggtgtttgcccgaaaatcctgcagcgttgcnnknnknnknnknnktgcccgggtgcttgcatctgccgtggtaacggttactgcatcacgggagatgcactagtaaa

SEQ ID NO: 143 - protein, 48 aa, Momordica cochinchinensis
  MOD_RES (23)..(27): Any amino acid
KEFHMCGSGSDGGVCPKILQRCXXXXXCPGACICRGNGYCITGDALVK

SEQ ID NO: 144 - DNA, 20 bp, Artificial Sequence
  source: /note=”Description of Artificial Sequence Synthetic primer“
aaagaattccatatgtgcgg

SEQ ID NO: 145 - DNA, 31 bp, Artificial Sequence
  source: /note=”Description of Artificial Sequence Synthetic primer“
tttactagtgcatctcccgtgatgcagtaac

SEQ ID NO: 146 - protein, 34 aa, Artificial Sequence
  source: /note=”Description of Artificial Sequence Synthetic polypeptide“
CGSGSDGGVCPKILQRCLRNKECPGACICRGNGY

SEQ ID NO: 147 - protein, 34 aa, Artificial Sequence
  source: /note=”Description of Artificial Sequence Synthetic polypeptide“
CGSGSDGGVCPKILQRCAEQWGCPGACICRGNGY

SEQ ID NO: 148 - protein, 34 aa, Artificial Sequence
  source: /note=”Description of Artificial Sequence Synthetic polypeptide“
CGSGSDGGVCPKILQRCFTPTNCPGACICRGNGY

SEQ ID NO: 149 - protein, 34 aa, Artificial Sequence
  source: /note=”Description of Artificial Sequence Synthetic polypeptide“
CGSGSDGGVCPKILQRCSPQLQCPGACICRGNGY

SEQ ID NO: 150 - protein, 34 aa, Artificial Sequence
  source: /note=”Description of Artificial Sequence Synthetic polypeptide“
CGSGSDGGVCPKILQRCRWREVCPGACICRGNGY

SEQ ID NO: 151 - protein, 34 aa, Artificial Sequence
  source: /note=”Description of Artificial Sequence Synthetic polypeptide“
CGSGSDGGVCPKILQRCRLDVLCPGACICRGNGY

SEQ ID NO: 152 - protein, 34 aa, Artificial Sequence
  source: /note=”Description of Artificial Sequence Synthetic polypeptide“
CGSGSDGGVCPKILQRCVAIRWCPGACICRGNGY

SEQ ID NO: 153 - protein, 34 aa, Artificial Sequence
  source: /note=”Description of Artificial Sequence Synthetic polypeptide“
CGSGSDGGVCPKILQRCYVQAGCPGACICRGNGY

SEQ ID NO: 154 - protein, 34 aa, Artificial Sequence
  source: /note=”Description of Artificial Sequence Synthetic polypeptide“
CGSGSDGGVCPKILQRCYLQLQCPGACICRGNGY

SEQ ID NO: 155 - protein, 34 aa, Artificial Sequence
  source: /note=”Description of Artificial Sequence Synthetic polypeptide“
CGSGSDGGVCPKILQRCGRCSVCPGACICRGNGY

SEQ ID NO: 156 - protein, 34 aa, Artificial Sequence
  source: /note=”Description of Artificial Sequence Synthetic polypeptide“
CGSGSDGGVCPKILQRCTQTLGCPGACICRGNGY

SEQ ID NO: 157 - protein, 34 aa, Artificial Sequence
  source: /note=”Description of Artificial Sequence Synthetic polypeptide“
CGSGSDGGVCPKILQRCQVFVTCPGACICRGNGY

SEQ ID NO: 158 - protein, 34 aa, Artificial Sequence
  source: /note=”Description of Artificial Sequence Synthetic polypeptide“
CGSGSDGGVCPKILQRCEVPCPCPGACICRGNGY

SEQ ID NO: 159 - protein, 34 aa, Artificial Sequence
  source: /note=”Description of Artificial Sequence Synthetic polypeptide“
CGSGSDGGVCPKILQRCLQRARCPGACICRGNGY

SEQ ID NO: 160 - protein, 34 aa, Artificial Sequence
  source: /note=”Description of Artificial Sequence Synthetic polypeptide“
CRSGSDGGVCPKILQRCPRTKGCPGACICRGNGY

SEQ ID NO: 161 - protein, 34 aa, Artificial Sequence
  source: /note=”Description of Artificial Sequence Synthetic polypeptide“
CGSGSDGGVCPKILQRCFRLAKCPGACICRGNGY

SEQ ID NO: 162 - protein, 34 aa, Artificial Sequence
  source: /note=”Description of Artificial Sequence Synthetic polypeptide“
CGSGSDGGVCPKILQRCHHPIXCPGACICRGNGY

SEQ ID NO: 163 - protein, 34 aa, Artificial Sequence
  source: /note=”Description of Artificial Sequence Synthetic polypeptide“
CGSGSDGGVCPKILQRCKGNNMCPGACICRGNGY

SEQ ID NO: 164 - protein, 34 aa, Artificial Sequence
  source: /note=”Description of Artificial Sequence Synthetic polypeptide“
CGSGSDGGVCPKILQRCLSLLKCPGACICRGNGY

SEQ ID NO: 165 - protein, 34 aa, Artificial Sequence
  source: /note=”Description of Artificial Sequence Synthetic polypeptide“
CGSGSDGGVCPKILQRCRDARECPGACICRGNGY

SEQ ID NO: 166 - protein, 34 aa, Artificial Sequence
  source: /note=”Description of Artificial Sequence Synthetic polypeptide“
CGSGSDGGVCPKILQRCQVSAPCPGACICRGNGY

SEQ ID NO: 167 - protein, 34 aa, Artificial Sequence
  source: /note=”Description of Artificial Sequence Synthetic polypeptide“
CGSGSDGGVCPKILQRCNVRPCCPGACICRGNGY

SEQ ID NO: 168 - protein, 34 aa, Artificial Sequence
  source: /note=”Description of Artificial Sequence Synthetic polypeptide“
CGSGSDGGVCPKILQRCCRSVXCPGACICRGNGY

SEQ ID NO: 169 - protein, 34 aa, Artificial Sequence
  source: /note=”Description of Artificial Sequence Synthetic polypeptide“
CGSGSDGGVCPKILQRCLNSMNCPGACICRGNGY

SEQ ID NO: 170 - protein, 34 aa, Artificial Sequence
  source: /note=”Description of Artificial Sequence Synthetic polypeptide“
CGSGSDGGVCPKILQRCKRISMCPGACICRGNGY

SEQ ID NO: 171 - protein, 34 aa, Artificial Sequence
  source: /note=”Description of Artificial Sequence Synthetic polypeptide“
CGSGSDGGVCPKILQRCEEYRPCPGACICRGNGY

SEQ ID NO: 172 - protein, 34 aa, Artificial Sequence
  source: /note=”Description of Artificial Sequence Synthetic polypeptide“
CGSGSDGGVCPKILQRCRCSGRCPGACICRGNGY

SEQ ID NO: 173 - protein, 34 aa, Artificial Sequence
  source: /note=”Description of Artificial Sequence Synthetic polypeptide“
CGSGSDGGVCPKILQRCSRLMGCPGACICRGNGY

SEQ ID NO: 174 - protein, 34 aa, Artificial Sequence
  source: /note=”Description of Artificial Sequence Synthetic polypeptide“
CGSGSDGGVCPKILQRCTKWRRCPGACICRGNGY

SEQ ID NO: 175 - protein, 34 aa, Artificial Sequence
  source: /note=”Description of Artificial Sequence Synthetic polypeptide“
CGSGSDGGVCPKILQRCNVFGSCPGACICRGNGY

SEQ ID NO: 176 - protein, 34 aa, Artificial Sequence
  source: /note=”Description of Artificial Sequence Synthetic polypeptide“
CGSGSDGGVCPKILQRCALXMSCPGACICRGNGY

SEQ ID NO: 177 - protein, 34 aa, Artificial Sequence
  source: /note=”Description of Artificial Sequence Synthetic polypeptide“
CGSGSDGGVCPKILQRCLWMRPCPGACICRGNGY

SEQ ID NO: 178 - protein, 34 aa, Artificial Sequence
  source: /note=”Description of Artificial Sequence Synthetic polypeptide“
CGSGSDGGVCPKILQRCCCYITCPGACICRGNGY

SEQ ID NO: 179 - protein, 34 aa, Artificial Sequence
  source: /note=”Description of Artificial Sequence Synthetic polypeptide“
CGSGSDGGVCPKILQRCSHLRICPGACICRGNGY

SEQ ID NO: 180 - protein, 34 aa, Artificial Sequence
  source: /note=”Description of Artificial Sequence Synthetic polypeptide“
CGSGSDGGVCPKILQRCHTRGECPGACICRGNGY

SEQ ID NO: 181 - protein, 34 aa, Artificial Sequence
  source: /note=”Description of Artificial Sequence Synthetic polypeptide“
CGSGSDGGVCPKILQRCRDPVGCPGACICRGNGY

SEQ ID NO: 182 - protein, 34 aa, Artificial Sequence
  source: /note=”Description of Artificial Sequence Synthetic polypeptide“
CGSGSDGGVCPKILQRCDDIFSCPGACICRGNGY

SEQ ID NO: 183 - protein, 34 aa, Artificial Sequence
  source: /note=”Description of Artificial Sequence Synthetic polypeptide“
CGSGSDGGVCPKILQRCKLSVGCPGACICRGNGY

SEQ ID NO: 184 - protein, 34 aa, Artificial Sequence
  source: /note=”Description of Artificial Sequence Synthetic polypeptide“
CGSGSDGGVCPKILQRCDTPXDCPGACICRGNGY

SEQ ID NO: 185 - protein, 34 aa, Artificial Sequence
  source: /note=”Description of Artificial Sequence Synthetic polypeptide“
CGSGSDGGVCPKILQRCTKPGRCPGACICRGNGY

SEQ ID NO: 186 - protein, 34 aa, Artificial Sequence
  source: /note=”Description of Artificial Sequence Synthetic polypeptide“
CGSGSDGGVCPKILQRCTRAPRCPGACICRGNGY

SEQ ID NO: 187 - protein, 34 aa, Artificial Sequence
  source: /note=”Description of Artificial Sequence Synthetic polypeptide“
CGSGSDGGVCPKILQRCRLVAHCPGACICRGNGY

SEQ ID NO: 188 - protein, 34 aa, Artificial Sequence
  source: /note=”Description of Artificial Sequence Synthetic polypeptide“
CGSGSDGGVCPKILQRCEDMGVCPGACICRGNGY

SEQ ID NO: 189 - protein, 34 aa, Artificial Sequence
  source: /note=”Description of Artificial Sequence Synthetic polypeptide“
CGSGSDGGVCPKILQRCLCLLRCPGACICRGNGY

SEQ ID NO: 190 - protein, 34 aa, Artificial Sequence
  source: /note=”Description of Artificial Sequence Synthetic polypeptide“
CGSGSDGGVCPKILQRCSDTSHCPGACICRGNGY

SEQ ID NO: 191 - protein, 34 aa, Artificial Sequence
  source: /note=”Description of Artificial Sequence Synthetic polypeptide“
CGSGSDGGVCPKILQRCPAERGCPGACICRGNGY

SEQ ID NO: 192 - protein, 34 aa, Artificial Sequence
  source: /note=”Description of Artificial Sequence Synthetic polypeptide“
CGSGSDGGVCPKILQRCSGARLCPGACICRGNGY

SEQ ID NO: 193 - protein, 34 aa, Artificial Sequence
  source: /note=”Description of Artificial Sequence Synthetic polypeptide“
CGSGSDGGVCPKILQRCIEKPXCPGACICRGNGY

SEQ ID NO: 194 - protein, 34 aa, Artificial Sequence
  source: /note=”Description of Artificial Sequence Synthetic polypeptide“
CGSGSDGGVCPKILQRCFTLLLCPGACICRGNGY

SEQ ID NO: 195 - protein, 34 aa, Artificial Sequence
  source: /note=”Description of Artificial Sequence Synthetic polypeptide“
CGSGSDGGVCPKILQRCYTAWVCPGACICRGNGY

SEQ ID NO: 196 - protein, 34 aa, Artificial Sequence
  source: /note=”Description of Artificial Sequence Synthetic polypeptide“
CGSGSDGGVCPKILQRCSRPVPCPGACICRGNGY

SEQ ID NO: 197 - protein, 34 aa, Artificial Sequence
  source: /note=”Description of Artificial Sequence Synthetic polypeptide“
CGSGSDGGVCPKILQRCRNPRGCPGACICRGNGY

SEQ ID NO: 198 - protein, 34 aa, Artificial Sequence
  source: /note=”Description of Artificial Sequence Synthetic polypeptide“
CGSGSDGGVCPKILQRCLADRHCPGACICRGNGY

SEQ ID NO: 199 - protein, 34 aa, Artificial Sequence
  source: /note=”Description of Artificial Sequence Synthetic polypeptide“
CGSGSDGGVCPKILQRCXTMSRCPGACICRGNGY

(19) United States
(12) Patent Application Publication
(10) Pub. No.: US 2012/0283136 A1
(43) Pub. Date: Nov. 8, 2012

(54) COMPOSITIONS AND METHODS FOR THE RAPID BIOSYNTHESIS AND IN VIVO SCREENING OF BIOLOGICALLY RELEVANT PEPTIDES

(75) Inventor: Julio A. Camarero, Los Angeles, CA (US)

(73) Assignee: THE UNIVERSITY OF SOUTHERN CALIFORNIA

(21) Appl. No.: 13/379,340

(22) PCT Filed: Jun. 23, 2010
(86) PCT No.: PCT/US10/39720

§ 371 Date: May 7, 2012

Related U.S. Application Data
(60) Provisional application No. 61/220,169, filed on Jun. 24, 2009.

(51) Int. Cl. C40B 30/06; G01N 21/64; G01N 21/76; C12N 1/21; C12N 5/10; C12N 1/19; C12N 1/15; C12N 1/13; C12Q 1/68; C12N 15/63

(52) U.S. Cl. 506/10; 435/6.13; 435/320.1; 435/325; 435/348; 435/419; 435/363; 435/254.2; 435/254.11; 435/354; 435/353; 435/350; 435/349; 435/366; 435/257.2; 435/252.31; 435/252.33; 435/252.3; 435/351

(57) ABSTRACT

This invention provides new combinatorial approaches for the biosynthesis and screening of cyclic peptides inside living cells. These novel approaches are useful for finding biologically relevant molecules, e.g., those able to inhibit the cytotoxicity of Anthrax Edema Factor. Key to this ‘living combinatorial’ approach is the use of a living cell as a micro-chemical factory for both synthesis and screening of potential inhibitors for a given molecular recognition event.

### CROSS-REFERENCE TO RELATED APPLICATIONS

This application claims priority under 35 U.S.C. §119(e) to U.S. Provisional Application Ser. No. 61/220,169, filed Jun. 24, 2009, the contents of which are hereby incorporated by reference into the present disclosure.

### STATEMENT OF GOVERNMENT SUPPORT

This invention was made with government support under Grant No. R01 GM090323-01 awarded by the National Institutes of Health. The government has certain rights in the invention.

### BACKGROUND

In order to be useful, high-throughput screens should be fast—allowing fast and efficient analysis in a short period of time. With respect to available screens for biologically relevant molecules that facilitate or impede protein-protein interactions, the available methods for solving this daunting problem are either based on rational or combinatorial approaches. The rational approach uses the molecular structure of the target to be knocked out, and then using docking software is able to find potential binders within a virtual library of small organic molecules. Despite the big advances in computing technology, however, this is still a slow process yielding around one ligand per year. Furthermore, most of the docking software available is based on static docking programs which reduces considerably the efficiency of this technique. Combinatorial approaches, on the other hand, use a random approach to generate as many compounds as possible with the hope that some of the members in these huge mixtures of compounds (also called libraries) will have activity against the molecular target (this is a common approach in nature, active antibodies are created and selected in that way). Combinatorial libraries can be generated using chemical or biological tools. Chemical libraries are usually generated in solid-phase and consequently the libraries are limited to contain 10⁶ different compounds as maximum. Biological libraries can reach up to 10⁹ members per library (as in phage display technology), however, they are mostly limited to the generation of peptide/proteins libraries only. In both cases, however, the main limitation is the screening process, which is carried in vitro and is time consuming.

### SUMMARY OF THE INVENTION

This invention relates to the development of a new combinatorial approaches for the biosynthesis and screening of cyclic peptides inside living cells. These novel approaches are useful for finding biologically relevant molecules, e.g., those able to inhibit the cytotoxicity of Anthrax Edema Factor. Key to this ‘living combinatorial’ approach is the use of a living cell as a micro-chemical factory for both synthesis and screening of potential inhibitors for a given molecular recognition event. The great advantage of this approach is that all the processes (i.e. biosynthesis of the library and screening) happen in the cell and therefore no in vitro screening is required. This considerably speeds up the whole process. Furthermore, because the screening process is taking place in a complex media composed by thousand of proteins (i.e. inside the cytoplasma's cell) it is expected that only members of the library with high specificity for the target will be selected. This will minimize the selection of universal binders, a real problem when in vitro screening methods are employed.

In summary, the development of this novel approach introduces a generic technology for fast and efficient identification of high-affinity ligands that can be used as effective countermeasures to biological threats.

Unique to this ‘living combinatorial’ approach is the use of a living cell as a micro-chemical factory for both synthesis and screening of potential inhibitors for a given molecular recognition event. This technique has the advantage that both processes synthesis and screening happen inside the cell thus accelerating the whole process of selection. Most of the combinatorial approaches developed so far to screen biological libraries rely on in vitro screening processes that are very time consuming and prone to select binders with poor specificity.

A recombinant peptide for use for the manufacture of peptide libriaries is also provided herein. This peptide comprises, or alternatively consists essentially of, or yet further consist of, a peptide template to be cyclized fused to an engineered C-intein fused to the N-terminal of the peptide template and an engineered N-intein fused to the C-terminal of the peptide template. As used herein, the term “peptide template” intends a protein or protein fragments that in one aspect, is an agent that is or may facilitate or inhibit protein-protein interactions in a cell. The peptide templates are one member of a peptide library that is screened for biologically relevant molecules or peptides. The peptides may optionally further comprise, or alternatively consists essentially of, or yet further, consists of, a label or tag, e.g., a fluorescent tag or a CBD affinity tag. Polynucleotides encoding the polypeptides are further provided herein as well as use of the composition for screening for biologically relvent agents such as peptides or small molecules.

This invention also provides an isolated host cell having one or more peptides as described herein. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

This invention also provides an isolated host cell having one or more polynucleotide encoding the polypeptides as described herein. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

### BRIEF DESCRIPTION OF THE FIGURES

FIG. 1 graphically shows the principle for the cell-based screening of genetically-encoded libraries of cyclotides for interfering/binding to particular protein interactions/protein targets. This approach proceeds a high through platform for the selection of a new set of highly stable cyclotide-based protein capture reagents.

FIGS. 2A and 2B illustrate the following: A. Primary and tertiary structure of MCoTI and Kalata cyclotides isolated from Momordica cochinchinensis and Oldenlandia affinis, respectively. Heitz et al. (2001) Biochemistry 40(27):7973-7983; Saether et al. (1995) Biochemistry. 34(13):4147-4158; Felizmenio-Quimio et al. (2001) J. Biol. Chem. 276(25):22875-22882. B. Multiple sequence alignment of cyclotide MCoTI-I with other squash trypsin inhibitors. Multiple sequence alignment was performed using TCoffee (available at the web address ca.expasy.org/cgi-bin/hub, last accessed Jun. 21, 2010) and visualized using Jalview.

FIG. 3 graphically shows biosynthetic approach for in vivo production of cyclotides Kalata B1 and MCoTI-II inside live E. coli cells. Backbone cyclization of the linear precursor is mediated by a modified protein splicing unit or intein. The cyclized product then folds spontaneously in the bacterial cytoplasm.

FIG. 4 graphically shows the principle for the cell-based screening of biosynthesized genetically-encoded cyclotide libraries using a FRET-based approach. See Table 1 for proteins used as A and B.

FIG. 5 graphically shows the principle for the cell-based screening of biosynthesized cyclotides using a lethality-based screening approach. Key is the use of conditional protein splicing through the interaction between proteins A and B to produce full-length Barnase. See Table 1 for proteins used as A and B.

FIG. 6 shows a three-dimensional structure of Bacillus amyloliquefaciens Barnase showing the ligation site that can be used to reconstitute the active enzyme through conditional protein trans-splicing. The residues forming the active site are Arg, His and Lys in the center of the structure. The fragments that can be used as N- and C-extein polypeptides are from about amino acid 30 to about amino acid 66, or from about amino acid 67 to about 110, respectively. The ligation site is located in a solvent-exposed loop away form the active site. Furthermore, multiple sequence alignment of different Barnases from several Bacillus strains shows that this site may tolerate well the introduction of the mutations required to facilitate protein trans-splicing.

FIG. 7 shows site-specific and traceless immobilization of proteins onto solid supports through protein trans-splicing. Maltose binding protein (MPB) was directly immobilized without performing any purification or reconcentration step from a) soluble cellular fraction of E. coli cells overexpressing MBP-IN, and b) MBP-IN expressed in vitro using a cell-free expression system. MBP was detected by immunofluorescence. MBP-IN concentration on the different samples spotted was estimated by Western-blot analysis.

FIGS. 8A and B show the secondary structures of EF alone (FIG. 8A) and complexed with CaM in the presence of an ATP substrate analogue (FIG. 8B). CaM is held in one side by C_{A} and in the other side by switch C and the helical domain. The substrate analogue and metal are bound between domains C_{A} and C_{B}, which form the active site. McPherson, J. D. et al. (2001) Nature 409(6822):934-941.

FIG. 9 is a comparison of CaM conformations in different CaM-effector complexes (A-D). The C-terminal and N-terminal domain of CaM are (I to IV) and (IV′ to VII), respectively. The effector poly-peptides are in outlined and boxed. E, Secondary structure of CaM with Ca²⁺-binding sites are identified as circled discs and as Sites 1 through 4. Figure modified from McPherson et al. (2001) Nature 409(6822):934-941.

FIG. 10A shows the principle of Native Chemical Ligation (NCL).

FIG. 10B shows that intramolecular NCL leads to the formation of backbone cyclized polypeptides.

FIGS. 11A and B schematically show biosysnthesis of recombinant polypeptide alpha-thioesters using protein splicing tools. FIG. 11A is a scheme representing the proposed mechanism for protein trans-splicing. FIG. 11B shows expression, purification and cleavage of a polypeptide-intein-CBD fusion protein (the asterisk refers to the mutation of the last residue of the intein, Asn to Ala and CBD refers to a chitin binding domain) with a soluble thiol.

FIG. 12 show biosynthetic approach for the in vivo production of cyclotides in E. coli cells.

FIG. 13A shows the primary structure and disulfide connectivity of cyclotide KB1.

FIG. 13B shows the sequences of the different linear precursors that will be used for the backbone cyclization of cyclotide KB1. A similar approach can be used for cyclotide MCoTI-II.

FIG. 14 is a schematic strategy for assembling a double stranded DNA coding for a full-length cyclotide with degenerated inserts for loops 2 and 5.

FIG. 15 shows the mechanism of protein splicing in cis (left panel) and in trans (righ panel).

FIGS. 16A and B illustrate principles of conditional protein splicing (CPS). FIG. 16A illustrates the conditional protein trans-splicing between two complementary intein halves can be promoted by interactions between two interacting proteins and results in the formation of a native peptide bond between the N- and C-extein polypeptides. FIG. 16B show crystal structure of the wild-type VMA intein, which is composed of the catalytic intein domain and the intervening endonuclease domain. Model showing the arrangement of the VMA intein and the EF-CaM complex.

FIGS. 17A and B show two protein constructs that can be generated and used in the in vivo screening approach for detecting antagonists for the EF-CaM complex.

FIG. 18 shows the structure of X. laevis MDM2 (grey surface) bound to the transactivation domain of human p53 (backbone representation, pdb access code: 1YCQ). McPherson, J. D. et al. (2001) Nature 409(6822):934-941. Key side-chains from p53 Phe19, Trp23 and Leu26 are also shown.

FIGS. 19A and 19B schematically shows biosynthesis of circular peptides which can be achieved by engineered protein splicing (FIG. 19A) or using protein trans-splicing (FIG. 19B).

FIGS. 20A and 20 B depict analytical reversed-phase HPLC traces of trypsin-bound fractions from MCoTI Lib1 and Lib2 libraries obtained in vitro by GSH-induced cleavage and folding. A. Total trypsin-bound fractions. Highlighted disk shows the position where mutant K4A should be eluting. B. Sequential fractions extracted during competitive trypsin-binding experiments.

FIG. 21 shows elution profiles for members of the MCoTI Lib1 and Lib2 extracted using trypsin-sepharose beads under competing conditions. The results shown are the average data obtained in vivo and in vitro (vertical bars indicate standard deviation). Quantification was done by integration of the corresponding HPLC peaks monitored at 220 nm (FIG. 20B). ES-MS was used to calculate the ratio of different cyclotides present in peaks with multiple products or not well resolved by HPLC. The area estimated for mutants with loss or gain of aromatic residues was corrected accordingly to take this into account.

FIG. 22 illustrates a summary of the relative affinities for trypsin of the different MCoTI-I mutants studied in this work. A model of cyclotide MCoTI-I bound to trypsin is shown at the bottom indicating the position of the mutations. The side-chain of residue Lys⁴ is shown in grey bound to specificity pocket of trypsin. The model was produced by homology modeling at the Swiss model workspace (available at the web address swissmodel.expasy.org//SWISS-MODEL.html, last accessed Jun. 21, 2010) using the structure of CPTI-II-trypsin complex (PDB code: 2btc) as template. Structure was generated using the PyMol software package.

FIG. 23 represents analysis by 4-20% gradient SDS-PAGE of the expression levels and in vivo cleavage of MCoTI-Lib2 precursors using different cellular backgrounds. Expression levels for MCoTI-Lib1 were similar. Induction was carried out at 30° C. (2 and 4 h) or 20° C. (20 h) by adding 0.3 mM IPTG.

FIG. 24 shows analytical reversed-phase HPLC trace of GSH-induced cyclization of MCoTI-Lib1 precursors before and after being purified by affinity chromatography on trypsin-sepharose. Identification of the different MCoTI-I mutants was carried out by ES-MS.

FIG. 25 depicts heteronuclear ¹H{¹⁵N} HSQC-spectra of recombinant wt MCoTI-I wt (black) and K4A mutant (grey). Chemical shift assignments of the wt MCoTI-I amino acid residues are shown in black. K4A mutant residues that changed chemical shifts by more that 0.3 ppm in proton dimension are grey. Small unassigned peaks in both wt and K4A spectra of MCoTI-I are from a minor isomer of the protein due to a known isomerization of the backbone at an Asp-Gly sequence in loop 6 of MCoTI-I. NMR experiments were acquired on a Bruker Avance II 700 MHz NMR spectrometer equipped with a cryoprove at 27° C. NMR samples of 0.2 mM of [U-, ¹⁵N] MCoTI-I and 0.25 mM of [U-, ¹⁵N] K4A MCoTI-I were in 90% H₂O/10% D₂O adjusted to pH 3.5 by addition of dilute HCl.

FIG. 26 illustrates analytical reversed-phase HPLC trace of GSH-induced cyclization of MCoTI-G25P precursor before and after being purified by affinity chromatography on trypsin-sepharose. Identification of the folded mutant was carried out by ES-MS.

FIG. 27 illustrates analytical reversed-phase HPLC trace of GSH-induced cyclization of MCoTI-I20G precursor before and after being purified by affinity chromatography on trypsin-sepharose. Identification of the folded mutant was carried out by ES-MS.

FIG. 28 shows analytical reversed-phase HPLC trace of GSH-induced cyclization of ¹⁵N labeled MCoTI-I wt and K4A precursors before and after being purified by preparative HPLC. Identification of the folded mutant was carried out by ES-MS. Expected molecular weight for the ¹⁵N-labeled cyclotide is shown in parenthesis.

FIG. 29 shows genetically encoded FRET reporters used in this example. Single letter codes are used to represent the LF recognition sequence and flexible linker. Expected masses were calculated for the mature proteins without N-terminal methionine.

FIG. 30 depicts expression and purification of FRET reporter protein 2. A. Gradient SDS-PAGE analysis of bacterial cell lysate expressing reporter 2 (line 1) and after purification by Ni-NTA affinity chromatography (line 2). B. ES-MS analysis of purified FRET reporter 2.

FIGS. 31A through D show in vitro cleavage of FRET reporter 1 to 5 by LF protease. A. Fluorescence spectra of a 10 nM solution of construct 3 incubated with LF (100 nM) at different time points. Excitation was done at 413 nm. B. Analysis of the proteolytic cleavage of construct 3 by gradient SDS-PAGE. C. Effect of the Gly-Gly-Ser linker length on the cleavage rate by LF. D. Fluorescence analysis of a 10 nM solution of construct 3 cleaved with different concentrations of LF protease. The FRET ratio change was calculated as described under Materials and Methods.

FIG. 32 illustrates the scheme employed for the production of a cell-based reporter for screening LF activity inside living bacterial cells.

FIGS. 33A and B show A. In vivo cleavage of FRET reporter 6 followed by fluorescence spectroscopy. A. Fluorescence spectra of E. coli cells expressing reporter 6 in the presence (lower line in FIG. 33A) or absence (upper line in FIG. 33A) of LF. B. Quantification of fluorescent protein YPet was performed on live E. coli cells expressing reporter 6 in the presence (upper line in FIG. 33B) or absence (lower line in FIG. 33B) of LF. Cells were excited at 490 nm.

FIG. 34 graphically displays FACS analysis of the of E. coli cells expressing FRET reporter in the presence (FRET⁺, LF⁺) or in the presence of LF Protease Inhibitor III (FRET⁺, LF⁻) of LF.

FIG. 35 depicts a scheme used for the cloning of cyclotide-based libraries using the pBAD24 derived expression vector.

FIGS. 36A and 36B illustrate the molecular approach to build MCoTI-I-based genetically encoded libraries using loop 2 (A) into orthogonal plasmids pTXB1 and pBAD24-Intein (B).

FIG. 37 shows a sequence of clones isolated from a MCoTI-I loop 2 based library using the pTXB1 plasmid. Single code letters are used for the amino acids in the sequence. A dot represents an amber stop codon.

FIG. 38 depicts HPLC analytical traces of the cyclization/folding crudes for individual clones isolated from the MCoTI-I loop 2 library expressed using plasmid pTXB1 in E. coli before and after purification using trypsin-agarose beads. Folded cyclotides were correctly characterized by ES-MS (mass spectrometry) and their ability to bind trypsin.

### DETAILED DESCRIPTION OF THE EMBODIMENTS

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

### DEFINITIONS

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

The term “isolated” as used with respect to cells, in particular stem cells, such as mesenchymal stem cells, refers to cells separated from other cells or tissue that are present in the natural tissue in the body.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Innis et al. (1990) Mol. Cell. Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype” refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on May 21, 2008. Biologically equivalent polynucleotides are those having the specified percent homology and encoding a polypeptide having the same or similar biological activity.

The term “an equivalent nucleic acid or polynucleotide” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. For examples of carriers, stabilizers and adjuvants, see Sambrook and Russell (2001), supra. Those skilled in the art will know many other suitable carriers for binding polynucleotides, or will be able to ascertain the same by use of routine experimentation. In one aspect of the invention, the carrier is a buffered solution such as, but not limited to, a PCR buffer solution.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, a “vector” is a vehicle for transferring genetic material into a cell. Examples of such include, but are not limited to plasmids and viral vectors. A viral vector is a virus that has been modified to transduct genetic material into a cell. A plasmid vector is made by splicing a DNA construct into a plasmid. As is apparent to those of skill in the art, the appropriate regulatory elements are included in the vectors to guide replication and/or expression of the genetic material in the selected host cell.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral Vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells. Non-limiting examples include simian, bovine, ovine, porcine, murine, rats, canine, equine, feline, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to prokaryotic Cyanobacteria, bacillus bacteria, E. coli bacterium, and Salmonella bacterium.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionall contain detectable labels and are exemplified and described herein.

As used herein, the term “detectable label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarry system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “gene chips” or a “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

In one aspect, “gene chips” or “microarrays” containing probes or primers homologous to a polynucleotide described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the sequence(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

An “effective amount” is an amount sufficient to effect beneficial or desired results. An effective amount can be administered in one or more administrations, applications or dosages and can be empirically determined by those of skill in the art.

A “control” is an alternative subject or sample used in an experiment for comparison purpose. A control can be “positive” or “negative”. For example, where the purpose of the experiment is to determine a correlation of a mutated allele with a particular phenotype, it is generally preferable to use a positive control (a sample from a subject, carrying such mutation and exhibiting the desired phenotype), and a negative control (a subject or a sample from a subject lacking the mutated allele and lacking the phenotype).

### Descriptive Embodiments

In one aspect, a method is provided for identifying or determining if a test agent inhibits formation of a biologically relevant complex in vivo in a cell, wherein the cell comprises, or alternatively consists essentially of, or yet further consists of, a template plasmid or vector comprising a first discreet origin of replication operatively linked to a recombinant polynucleotide encoding a N-terminal leader sequence to generate an N-terminal Cys residue, a peptide template to be cyclized and an intein modified to generate a C-terminal thioester in vivo and a reporter plasmid or vector comprising a second discreet origin of replication operatively linked to one or more interacting domains and a lethality reporter and/or a detectable label, and where the method comprises culturing the cell under conditions to express the peptide template and subsequently culturing the cell under conditions to express the reporter plasmid or vector; and selecting cells, thereby determining if a test agent inhibits formation of a biologically relevant complex in vivo in the cell. In one aspect, the reporter plasmid or vector and/or the template plasmid or vector further comprises, or alternatively consists essentially of a drug resistant gene, that may be the same or different for the template and reporter plasmid or vector. Non-limiting examples of drug resistant genes are antibiotic resistant genes identified in Table 2. As used herein, the term “discrete” intends that the promoter, marker and origin of replication allow the differentiation of the protein when expressed in a cell. As is to be understood by those of skill in the art, these functional components are positioned within the recombinant peptide to be operatively linked to the sequences that they regulate. As is apparent to those skilled in the art, the method can further comprise, or alternatively consist essentially of, or yet further consist of, culturing a positive and negative control cell under the same conditions as the cell described herein.

As used herein, the term “interacting domain” intends a biological agent such as a polypeptide or protein with natural affinity for another. Non-limiting examples of such are an receptor and its ligand, an epitope and its binding domain, or an two interacting domains in a cellular pathway. Non-limiting examples of such are identified in Table 1. Further examples include a VMA-N-intein or a fragment or an equivalent of each thereof, e.g., the VMA-N-intein comprises amino acids 1 to 184 of the intein. The other interacting domain comprises, or alternatively consists essentially of, or yet further consists of, a VMA-C intein or a fragment or an equivalent of each thereof, e.g., the VMA-C intein unit comprises amino acids 390 to 454 of the VMA-C intein. In a further aspect, the VMA-N intein and/or VMA-C intein further comprises, or alternatively consists essentially of, or yet further consists of, a peptide of the group: a transactivation domain of p53; a adenylate cyclase domain of EF or a CaM protein that binds the adenylate cyclase domain of EF or a fragment or an equivalent of each thereof.

As used herein, an “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

In one aspect, the modified intein comprises, or alternatively consists essentially of, or yet further consists of, a Gyrase or VMA intein, an equivalent or a fragment of each thereof.

In another aspect, the peptide template to be cyclized further comprises, or alternatively consists essentially of, or yet further consists of, a discrete protein binding domain for isolation of the a template plasmid or vector from the cell.

As used herein, the term “peptide template” intends a protein or protein fragments that in one aspect, is an agent that is or may facilitate or inhibit protein-protein interactions in a cell. The peptide templates are one member of a peptide library that is screened for biologically relevant molecules or peptides. The peptides may optionally further comprise, or alternatively consists essentially of, or yet further, consists of, a discrete protein binding domain is different from the interacting domain and is utilized to isolate the template or polypeptide from the host cell. a label or tag, e.g., a fluorescent tag or a CBD affinity tag. In another aspect, the N-terminal leader sequence comprises, or alternatively consists essentially of, or yet further consists of, a leader sequence from the group of methionine, ubiquitin, modified ubiquitin or an equivalent of each thereof.

In a further aspect, the lethality reporter comprises, or alternatively consists essentially of, or yet further consist of, a recombinant polynucleotide encoding Barnase polypeptide or a fragment or an equivalent thereof.

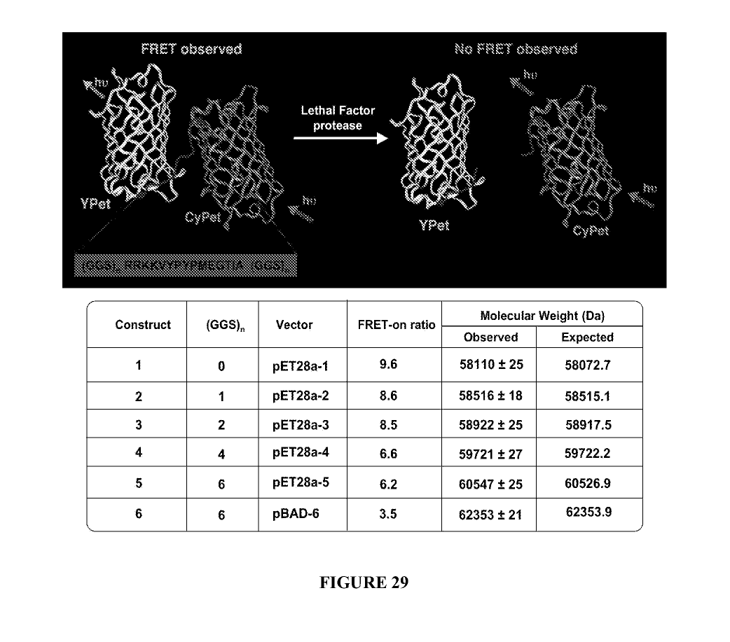

Non-limiting examples of detectable labels for use in the method comprise a fluorescent label and such as FRET reporters, e.g., CyPet and YPet and equivalents of each thereof. See Example 5 and FIG. 29.

In another aspect, the reporter plasmid or vector comprises a first plasmid or vector comprising, or alternatively consisting essentially of, or yet further consists of, a fragment of the detectable label fused at the C-terminus or N-terminus of one of the two the interacting domains and a second plasmid or vector comprising a detectable label fused to the N-terminus or C-terminus of the other interacting domain, wherein the first and the second detectable label or fragments thereof emit a detectable signal when brought into proximity with each other by the binding or fusion of the interacting domains.

The two interacting domains can be contained within discreet plasmid or vectors or alternatively within a a polycistronic vector. The regulatory and other elements of the discreet plasmids or vectors can be independently the same or different for optimal control over expression.

In a further aspect, the reporter plasmid or vector further comprises, or alternatively consists essentially of, or yet further consists of, a polynucleotide encoding a peptide linker between the interacting domain and the detectable label. Examples of such are provided herein.

In a yet further aspect, the reporter plasmid or vector comprises, or alternatively consists essentially of, or yet further consists of, a first plasmid or vector comprising a first fragment of the lethality reporter fused at the C-terminus or the N-terminus of one of the two interacting domains and a second plasmid or vector comprising a second fragment of the lethality reporter fused at the C-terminus or N-terminus of the second interacting domain, and wherein the first and second fragment of the lethality reporter will kill the host cell when brought into proximity with each other by the binding or fusing of the first and second interacting domains. The two interacting domains can be contained within discreet plasmid or vectors or alternatively within a a polycistronic vector. The regulatory and other elements of the discreet plasmids or vectors can be independently the same or different for optimal control over expression. Moreover, the reporter plasmid or vector can further comprise, or alternatively consist essentially of, or yet further consist of, a polynucleotide encoding a peptide linker between the interacting domain and the detectable label. Non-limiting examples of peptide linkers are provided herein.

In a further aspect of the above embodiments, the template plasmid or vector and/or the reporter plasmid or vector further comprises, or alternatively consists essentially of, or yet further consists of, one or more of a discrete promoter or a discrete marker.

In one aspect, the cells are selected by selecting cells that survive or remain viable and/or express the detectable label. In another aspect, the cells are selected by selecting cells that do not survive or remain viable and/or do not express the detectable label. In a yet further aspect, the method further comprises, or alternatively consists essentially of, or yet further consists of, isolating and sequencing the peptide template from the cell.

The cells for use in the methods described herein are isolated host cells. “Host cell” refers not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Examples of such include, eukaryotic and prokaryotic cells such as E. coli cells. Examples of eukaryotic cells include, but are not limited to cells from animals, e.g., murines, rats, rabbit, simians, bovines, ovine, porcine, canines, feline, farm animals, sport animals, pets, equine, and primate, particularly human. The cells can be cultured cells or they can be primary cells. Cultured cell lines can be purchased from vendors such as the American Type Culture Collection (ATCC), U.S.A.

Further provided in this disclosure are the compositions for use in the methods described herein. In one aspect, provided is a template plasmid or vector comprising a first discreet origin of replication operatively linked to a recombinant polynucleotide encoding a N-terminal leader sequence to generate an N-terminal Cys residue, a peptide template to be cyclized and an intein modified to generate a C-terminal thioester in vivo. Also provided is a reporter plasmid or vector comprising a discreet origin of replication operatively linked to one or more interacting domains and a lethality reporter and/or a detectable label, and where the method comprises culturing the cell under conditions to express the peptide template and subsequently culturing the cell under conditions to express the reporter plasmid or vector; and selecting cells, thereby determining if a test agent inhibits formation of a biologically relevant complex in vivo in the cell. In one aspect, the reporter plasmid or vector and/or the template plasmid or vector further comprises, or alternatively consists essentially of a drug resistant gene, that may be the same or different for the template and reporter plasmid or vector. Non-limiting examples of drug resistant genes are antibiotic resistant genes identified in Table 2. As used herein, the term “discrete” intends that the promoter, marker and origin of replication allow the differentiation of the protein when expressed in a cell. As is to be understood by those of skill in the art, these functional components are positioned within the plasmid or vector to be operatively linked to the sequences that they regulate.

As used herein, the term “interacting domain” intends a biological agent such as a polypeptide or protein with natural affinity for another. Non-limiting examples of such are an receptor and its ligand, an epitope and its binding domain, or an two interacting domains in a cellular pathway. Non-limiting examples of such are identified in Table 1. Further examples include a VMA-N-intein or a fragment or an equivalent of each thereof, e.g., the VMA-N-intein comprises amino acids 1 to 184 of the intein. The other interacting domain comprises, or alternatively consists essentially of, or yet further consists of, a VMA-C intein or a fragment or an equivalent of each thereof, e.g., the VMA-C intein unit comprises amino acids 390 to 454 of the VMA-C intein. In a further aspect, the VMA-N intein and/or VMA-C intein further comprises, or alternatively consists essentially of, or yet further consists of, a peptide of the group: a transactivation domain of p53; a adenylate cyclase domain of EF or a CaM protein that binds the adenylate cyclase domain of EF or a fragment or an equivalent of each thereof.

In one aspect, the modified intein comprises, or alternatively consists essentially of, or yet further consists of, a Gyrase or VMA intein, an equivalent or a fragment of each thereof.

As used herein, the term “peptide template” intends a protein or protein fragments that in one aspect, is an agent that is or may facilitate or inhibit protein-protein interactions in a cell. The peptide templates are one member of a peptide library that is screened for biologically relevant molecules or peptides. The peptides may optionally further comprise, or alternatively consists essentially of, or yet further, consists of, a discrete protein binding domain is different from the interacting domain and is utilized to isolate the template or polypeptide from the host cell. a label or tag, e.g., a fluorescent tag or a CBD affinity tag.

In another aspect, the N-terminal leader sequence comprises, or alternatively consists essentially of, or yet further consists of, a leader sequence from the group of methionine, ubiquitin, modified ubiquitin or an equivalent of each thereof.

In a further aspect, the lethality reporter comprises, or alternatively consists essentially of, or yet further consist of, a recombinant polynucleotide encoding a Barnase polypeptide or a fragment or an equivalent or each thereof.

Non-limiting examples of detectable labels for use in the method comprise a fluorescent label and such as FRET reporters, e.g., CyPet and YPet and equivalents of each thereof. See Example 5 and FIG. 29.

In another aspect, the reporter plasmid or vector comprises a first plasmid or vector comprising, or alternatively consisting essentially of, or yet further consists of, a fragment of the detectable label fused at the C-terminus or N-terminus of one of the two the interacting domains and a second plasmid or vector comprising a detectable label fused to the N-terminus or C-terminus of the other interacting domain, wherein the first and the second detectable label or fragments thereof emit a detectable signal when brought into proximity with each other by the binding or fusion of the interacting domains.

The two interacting domains can be contained within discreet plasmid or vectors or alternatively within a a polycistronic vector. The regulatory and other elements of the discreet plasmids or vectors can be independently the same or different for optimal control over expression.

In a further aspect, the reporter plasmid or vector further comprises, or alternatively consists essentially of, or yet further consists of, a polynucleotide encoding a peptide linker between the interacting domain and the detectable label. Examples of such are provided herein.

In a yet further aspect, the reporter plasmid or vector comprises, or alternatively consists essentially of, or yet further consists of, a plasmid or vector comprising a first fragment of the lethality reporter fused at the C-terminus or the N-terminus of one of the two interacting domains. In another aspect, the reporter plasmid or vector comprises, or alternatively consists essentially of, or yet further consists of, a second or discreet fragment of a lethality reporter as compared to the first fragment described above, fused at the C-terminus or N-terminus of a second interacting domain as compared to the reporter plasmid or vector described above, and wherein the first and second fragment of the lethality reporter in the plasmids or vectors will kill the host cell when brought into proximity with each other by the binding or fusing of the first and second interacting domains when expressed in a host cell. The two interacting domains can be contained within discreet plasmids or vectors or alternatively within a a polycistronic vector. The regulatory and other elements of the discreet plasmids or vectors can be independently the same or different for optimal control over expression. Moreover, the reporter plasmid or vector can further comprise, or alternatively consist essentially of, or yet further consist of, a polynucleotide encoding a peptide linker between the interacting domain and the lethality reporter. Non-limiting examples of peptide linkers are provided herein.

In a further aspect of the above embodiments, the template plasmid or vector and/or the reporter plasmid or vector further comprises, or alternatively consists essentially of, or yet further consists of, one or more of a discrete promoter or a discrete marker.

Also proficed are isolated host cells comprising one or more of the plasmid or vector as described above, wherein the host cell is a eukaryotic cell or a prokaryotic cell. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Examples of such include, prokaryotic cells such as E. coli cells. Examples of eukaryotic cells include, but are not limited to cells from animals, e.g., murines, rats, rabbit, simians, bovines, ovine, porcine, canines, feline, farm animals, sport animals, pets, equine, and primate, particularly human. The cells can be cultured cells or they can be primary cells. Cultured cell lines can be purchased from vendors such as the American Type Culture Collection (ATCC), U.S.A.

In a further aspect, the isolated host cell only contains the reporter plasmid or vector and a small molecule. The cell is grown under conditions that favor expression of the reporter plasmid and is used to identify or determine if the small molecule affects the binding of the interacting domain of the reporter plasmid or vector. Thus, also provided herein is a method to identify or determine if the small molecule affects the binding of the interacting domain of the reporter plasmid or vector by growing the host cell under conditions that favor expression of the reporter plasmid and then screening for activity by monitoring for the detectable label or survival or death of the cell when the reporter plasmid or vector contains a lethality reporter.

Yet further provided is a kit for determining identifying or determining if a test agent inhibits formation of a biologically relevant complex in vivo in a cell comprising the template plasmid or vector and/or the reporter plasmid or vector and instructions for carrying out the method as described herein.

Having described the general concepts of this invention, the following illustrative examples are provided.

### Experiment No. 1

### High-Throughput Assays for Proteome Research

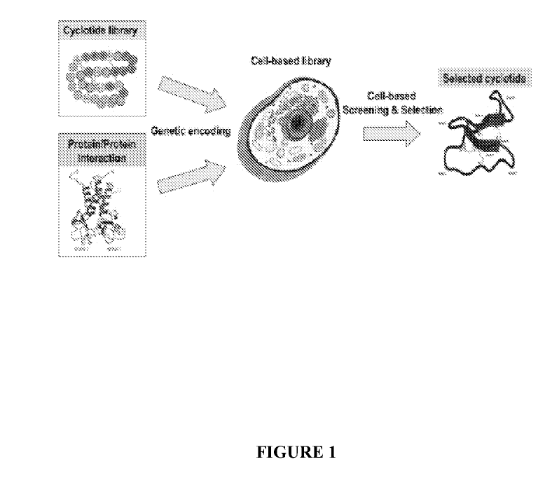

The development of high-through-put assays has been promoted again by the successful completion of complete genomes, including the human. McPherson et al. (2001) Nature 409(6822):934-941 and Venter et al. (2001) Science 291(5507):1304-1351. Of overarching importance for achieving this goal is the development of new protein capture tools for the detection and identification of specific proteins. These new set of capture reagents should be stable to thermal and proteolytic degradation, have high affinity, easy to produce and present low cross-reactivity. In response to this challenge, this invention provides the use of cell-based libraries of cyclotides for selecting specific cyclotide sequences against particular protein targets (FIG. 1).

Cyclotides are a new emerging family of plant-derived backbone-cyclized polypeptides (about 28-37 amino acids long) that share a disulfide-stabilized core (3 disulfide bonds) characterized by an unusual knotted structure. Their unique circular backbone topology and knotted arrangement of three disulfide bonds makes them exceptionally stable to thermal and enzymatic degradation. Cyclotides have been associated with a range of biological functions such as uterotonic activity, inhibition of trypsin and neurotensin binding, cytotoxicity, anti-HIV, antimicrobial, and insecticidal activity. Kimura et al. (2005) Protein Peptide Lett. 12(8):789-794. Together, these characteristics make cyclotides ideal candidates to be used as molecular scaffolds for the discovery of stable high affinity ligands against particular biomolecular targets, thus replacing the less stable antibody-based scaffold which have been traditionally used as the protein capture reagent of choice. Applicants have recently demonstrated the intriguing possibility of generating libraries of cyclotides inside living bacterial cells. Kimura et al. (2005) Protein Peptide Lett. 12(8):789-794; Camarero et al. (2007) Chembiochem. 45(6):973-976; Kimur et al. (2007) Anal. Biochem. 369(1):60-70; Kwon et al. (2006) Chem. Int. Ed. 45(11):1726-1729 and Camarero et al. (2007) Chembiochem. 8(12):1363-1366. Biologically-generated libraries can be then screened inside the cell using cell-based reporters based either on FRET (Kimura et al. (2007) Anal. Biochem. 369(1):60-70) or lethality for the selection of particular cyclotide sequences able to bind a particular protein target using high throughput methods such as fluorescence-activated cell sorting (FACS). Selected cyclotide sequences can be then immobilized using a micro-array format onto an appropriate solid support. Of particular interest is the use of protein trans-splicing (Kwon (2006) Angew. Chem. Int. Ed. 45(11):1726-1729), which was developed by Applicants, for the immobilization of these micro-proteins onto solid supports. This approach allows the site-specific and traceless immobilization of particular proteins/polypeptides (for example linearized cyclotides) from mixtures without any need to purify or re-concentrate the ligand to be immobilized. More importantly, this approach can be interfaced with cell free expression systems for the rapid and high throughput of specific cyclotide-based microarrays (Kwon (2006) Angew. Chem. Int. Ed. 45(11):1726-1729). In summary this invention presents an innovative approach for the screening and selection of a new type of extremely stable protein capture reagent in combination with a new versatile way of ligand immobilization for the rapid production of cyclotide-based micro-arrays. Cyclotide-based arrays have the unique potential to become in the future the reagents of choice for large scale parallel detection of proteins and facilitate comparative surveys of proteomes. Cyclotides share with protein scaffolds, such as antibodies, a stable three-dimensional structure and the ability to bind specifically to numerous proteins due to the presence of up to 5 hypervariable loops. However, in contrast with classical protein capture-reagents, these small proteins are extremely resistant to chemical and thermal denaturation, and extremely resistant to proteases, which will facilitate the analysis of complex biological mixtures such blood plasma serum, for example. Moreover, their small size also allows the rapid chemical synthesis and modification. Finally, the development by Applicants (Kimura et al. (2006) Angrew. Chem. Int. Ed. Engl. 45(6):973-976 and Camarero et al. (2007) Chembiochem. 8(12):1363-1366) of the only approach for the biosynthesis of folded cyclotides inside bacterial cells also facilitates the use of high throughput cell-based screening methods for selecting specific cyclotide sequences able to bind to any particular modified or unmodified protein or protein domain.

Applicants anticipate that the development of these technologies will bring protein micro-array technology to the point where could be routinely used for the analysis in masse of biomolecular interactions involving proteins. This high throughput ability of protein micro-arrays is in fact essential to the pharmaceutical industry and human health because most drugs used today are either proteins or alter the functions of proteins. Specific examples may include protein microarrays for mechanistic studies of drug action, monitoring antibodies contained in serum such as in the diagnostics of auto-immune diseases, and recombinant antibody library screening, etc.

The simultaneous identification and quantitative measurement of the production levels of thousands of different proteins in a biological specimen remains an unachieved goal of modern proteomic research. Reaching this goal depends on the development of new efficient protein capture tools able to reliably detect specific proteins. Ideally these protein capture-ligands should be able to detect a particular target protein over a broad range of concentrations and be able to distinguish post-translational modifications such as truncations, phosphorylation, acetylation, etc. Also of extreme importance, is the availability of suitable chemistries for the site-specific immobilization of such capture reagents onto solid-supports using a micro-array format, which will allow the high throughput analysis of this set of reagents. So far, the limited availability of suitable capture reagents as well as immobilization chemistries has been a major bottleneck to fully achieve this overarching goal. In response to this challenge a number the technologies are starting to emerge to replace antibodies as the protein-capture reagent of choice.

### Ultra-stable Micro-Protein Scaffolds as New Versatile Protein Capture Reagents

Special attention has been recently given to the use of highly constrained peptides, also known as micro- or miniproteins, as extremely stable and versatile scaffolds for the production of high affinity ligands for specific protein capture. Cyclotides are fascinating micro-proteins present in plants from the Violaceae, Rubiaceae and also Cucurbitacea and featuring various biological actions such as toxic, inhibitory, anti-microbial, insecticidal, cytotoxic, anti-HIV or hormone-like activity. Craik et al. (2006) Biopolymers 84(3):250-266 and Craik et al. (2002) Curr. Opin. Drug. Discov. Develop. 5(2):251-260.

They share a unique head-to-tail circular knotted topology of three disulfide bridges, with one disulfide penetrating through a macrocycle formed by the two other disulfides and inter-connecting peptide backbones, forming what is called a cystine knot topology. Cyclotides belong to the family of knottins, a group of microproteins which also is composed by conotoxins (389 sequences) and spider toxins (257 sequences), besides cyclotides. Basically, cyclotides are knottins with a head-to-tail circular topology. These micro-proteins are considered as natural combinatorial peptide libraries structurally constrained by the cystineknot scaffold (Craik et al. (2006) Biopolymers 84(3):250-266) and head-to-tail cyclization but in which hypermutation of essentially all residues are permitted with the exception of the strictly conserved cysteines of the knot. The main features of cyclotides and knotins in general are therefore a remarkable stability due to the cystine knot, a small size making them readily accessible to chemical synthesis, and an excellent tolerance to sequence variations.

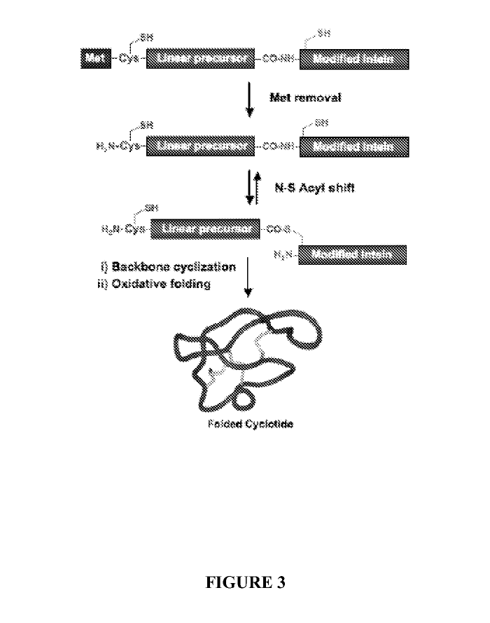

It is interesting to remark that the first cyclotide to be discovered, Kalata B1, has shown to be orally bioavailable. Saether et al. (1995) Biochemistry 34(13):4147-4158. Moreover, several cyclotides have been also shown to cross the cell membrane. Greenwood et al. (2007) Int. J. Biochem. Cell Biol. 39(12):2252-2264. Cylotides and knottins thus appear as appealing leads or frameworks for peptide drug design (Clark et al. (2006) Biochme. J. 394(Pt. 1):85-93 and Craik et al. (2006) Curr. Opin. Drug. Discov. Devel. 9(2):251-260) and as potential extremely versatile and stable protein capture reagents. Cyclotides are ribosomally produced in plants from precursors that comprise between one and three cyclotide domains, however the mechanism of excision of the cyclotide domains and ligation of the free N- and C-termini to produce the circular peptides has not been elucidated. Applicants have recently developed and successfully used a bio-mimetic approach for the biosynthesis of folded cyclotides inside cells by making use of modified protein splicing units (FIG. 3).

This important finding opens the intriguing possibility of generating large libraries of cyclotides (about 10⁹) for high throughput cell-based screening and selection of specific sequences able to recognize particular biomolecular targets. Camarero et al. (2007) Chembiochem. 8(12):1363-1366; Kimura et al. (2006) Angew. Chem. Int. Ed. Engl. 45(6):973-976 and Camarero et al. (2007) Chimicia Oggi/Chemistry Today 25(3):20-23. This invention combines this unique set of technologies for cell-based screening and selection of genetically-encoded libraries of cyclotides against a particular protein interaction or protein/protein domain.

### Competitor Approaches

### Antibody-Based Capture Reagents

Monoclonal antibodies have so far been the obvious choice of affinity-capture reagents for the construction of protein-detection chips. Indeed, several antibody-based chips have been described and antibody-coated surfaces are now commercially available. Furthermore, several thousand monoclonal antibodies have been isolated over the years that should (in principle) facilitate antibody-chip assembly. However, although IgG antibodies might offer some advantages such as multivalency, the need to generate monoclonal antibodies against thousands of different antigens might outpace the throughput capacity of hybridoma technology, which requires animal immunization and fairly laborious experimental procedures. Furthermore, IgG production in mammalian cell cultures might be a further limitation on the construction of antibody chips displaying many different binding specificities, due mainly to the difficulties associated with the selection of high affinity antibodies, cross-reactivity and stability. Cyclotides on the other hand are highly stable and have the potential to show high specificity against a variety of different protein targers due to the presence of up to 5 hyper-variable loops. They can also easily biosynthesized in bacterial cells and therefore can be readily interfaced with high throughput cell-based selection methods. Moreover its relatively small size (28-37 residues) makes them amenable to rapid chemical synthesis and modification.

### Aptamer Technology

Aptamers consisting of nucleic acids have been also used with more or less success to generate affinity capture chips. Osborne et al. (1997) Curre. Opin. Chem. Biol. 1(1):5-9. Nucleic acid (RNA or DNA) libraries usually are screened and binders selected using SELEX (selective evolution of ligands by exponential enrichment). The major criticism on using aptamers, however, is that they are susceptible to degradation by nucleases as well as the limited lack of chemical functionalities within the basic building blocks. Thus, meanwhile cyclotides and polypeptides in general have a repertoire of 20 amino acids; aptamers can only use 4 bases. Furthermore, cyclotides show a remarkable stability to harsh chemical conditions (low-high pH values, organic solvents) and proteases.

### Alternative Protein Scaffolds

The potential problems associated with the use of antibody fragments for chip assembly, which might manifest themselves through moderate expression yields (typically, 1-50 mg/L in shake flasks) and by issues related to the stability and solubility of these large proteins, have led to explore the use of alternative protein scaffolds as a source for new protein capture reagents. Kolmar et al. (2008) FEBS J. 275(11):2667. Proposed suitable protein scaffolds include fibronectin domains, the Z domain of protein A and lipocalins among others. High expression yields, the absence of disulfide bridges and relatively high thermodynamic stability compared to antibodies appear to be the most attractive features of these methods. However, the binding affinities and specificities obtained when using them still require extensive validation. Cyclotides (and knottins in general) on the other hand have already shown the potential to be used as capture reagents and therapeutic agents. Thus cyclotides have a diverse range of biological activities including uterotonic activity, anti-HIV, neurotensin inhibition, antimicrobial and insecticidal activity. Also, Ziconotide (PRIALT), a synthetic conotoxoin-derived synthetic peptide, is a neuroactive peptide already in the final stages of clinical development as a novel non-opioid treatment for severe chronic pain.

### Biosynthesis of Genetically-Encoded Libraries of Cyclotides

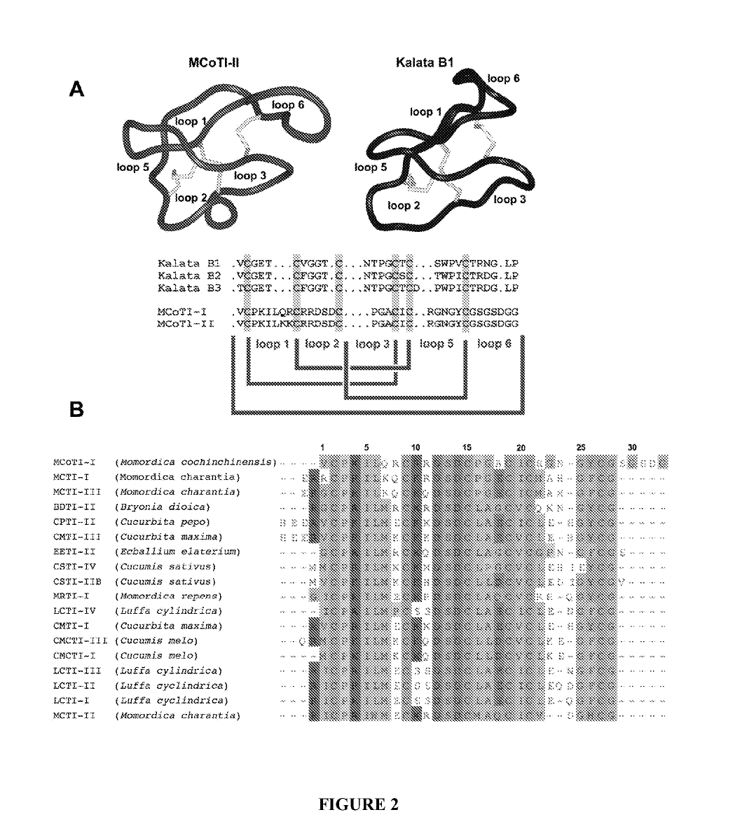

Cyclotide-based libraries are created at the DNA level using double stranded DNA inserts with degenerate sequences for some of the different loops of the cyclotide scaffold (i.e. loops 2, 3 and/or 5, see FIG. 2).

Among the different strategies that have been developed to produce clonable degenerate DNA sequences, Applicants use the method described by Scott and Smith. Scott and Smith (1990) Science 249(4967):386-390. This approach involves the generation of a double-stranded degenerate DNA by PCR. Briefly, a long degenerate synthetic oligonucleotide (which encodes the whole cyclotide, about 100 nt long) template is PCR amplified using 5′- and 3′-primers corresponding to the non degenerate flanking regions. The resulting double stranded degenerate DNA is double digested and then ligated to a linearized intein-containing expression vector to produce a library of plasmids. These libraries are then transformed into electrocompetent E. coli cells to finally obtain a library of cells containing typically up to about 10⁹ different clones (i.e. cyclotide sequences). The degenerate synthetic oligonucleotide template can be synthesized using a NN(G/T) codon scheme for the randomized loops. This scheme uses 32 codons to encode all 20 amino acids and encodes only 1 stop codon. Alternatively, the degenerate template can be also synthesized from mixtures of trinucleotide codons representing all 20 amino acids and no stop codons. It is anticipated that the first cyclotide-based libraries can be based on loops 2, 5 and/or 3 using the cyclotides KB1 and MCoTI-II as scaffolds. The complexity of these libraries will be around about 10⁶ and about 10⁹ members for combinations involving only one of the loops or two loops, respectively.

### Cell-Based Screening

Available methods for producing and screening high-affinity ligands against particular molecular targets are either based in rational or combinatorial approaches. The rational approach usually requires the molecular structure of the biomolecular target, and then potential binders are selected from a virtual library of compounds using docking software. Waszkowczy (2002) Opin. Drug Discov. Devel. 5(3):407-413. Despite recent advances in computing technology and the development of adaptive docking software (Waszkowczy (2002) Opin. Drug Discov. Devel. 5(3):407-413), this is still a slow (although promising) process. Combinatorial approaches, on the other hand, use a random generation of a large number of compounds that are then screened against a biomolecular molecular target. Most of the methods for library screening, however, are performed in vitro, which is a long and laborious process. Cell-based screening, on the other hand, opens the possibility of using single cells as microfactories where the biosynthesis and screening of particular ligand can take place in a single process within the same cellular cytoplasm. Kaganman (2007) Nat. Methods 4(2):112-113. The use of a complex molecular environment, such as the cellular cytoplasm, provides the ideal background for the selection of highly specific inhibitors. Furthermore, the recent introduction of genetically encoded fluorescence-based assays (Giepmans et al. (2006) Science 312(5771):217-224) allows the use of high-throughput screening methods such as fluorescence-activated cell sorting (FACS) for studying molecular interactions inside living cells. You et al. (2006) PNAS USA 103(49):18458-18463.

Thus, this invention provides the use two different cell-based screening approaches for the selection of particular cyclotide sequences able to inhibit or bind specific proteins. Applicant utilizes FRET-based cell-based screening reporter and recently developed a similar cell-based screening approach for Anthrax Lethal Factor antagonists. Kimura et al. (2007) Anal. Biochem. 369(1):60-70. It is important to remark that these cell-based assays using genetically-encoded reporters can be used to screen genetically-encoded libraries, but they can also be used to screen chemical libraries of organic compounds.

### FRET-Based In Vivo Screening Approach

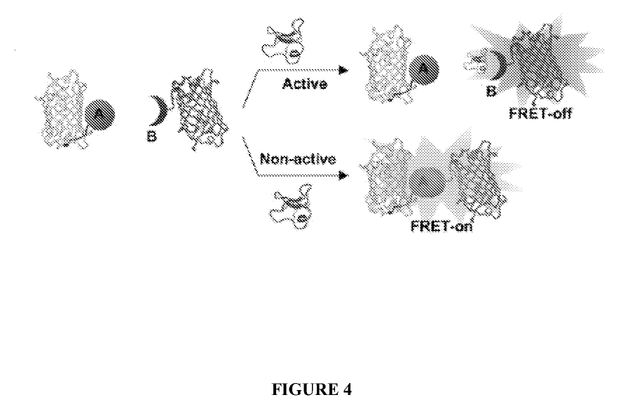

The CyPet and YPet fluorescent proteins are used as a FRET-couple to monitor protein interactions inside the cell. Kimura et al. (2007) Anal. Biochem. 369(1):60-70; You et al. (2006) PNAS USA 103(49):18458-18463. The principle for this approach is depicted in FIG. 4.

Briefly, the two proteins or protein domains mediating the interaction that to be inhibited will be fused to either the C-terminus or N-terminus of CyPet and YPet, respectively. In order to facilitate the interaction between targeted domains and prevent any steric hindrance that will interfere with the molecular recognition process, addition of appropriate flexible polypeptide linkers (i.e. [Gly-Gly-Ser]n), wherein n is an integer of 1 or greater, are also added at the junctions between the interacting proteins or protein domains and the corresponding fluorescent proteins. The DNA encoding for these constructs are cloned using the Duet family of vectors from Novagen. Multiple Duet vectors are used together in compatible host strains to co-express up to 8 target proteins (see Table 2 below). A similar approach also can be used to screen directly linearized cyclotide-based libraries against a particular target (see Table 1). You et al. (2006) PNAS USA 103(49):18458-18463 and Zhang et al. (2002) Nat. Rev. Mol. Cell. Bio. 3(12):906-918. In this case a linearized cyclotide-based library can be fused to one of the fluorescent proteins and the target protein to the other fluorescent protein. MCoTI-II and KB1 cyclotide based-libraries can be linearized at loop 6. It is interesting to remark that MCoTI-III, one of the other Trypsin cystine-knot peptide inhibitors isolated from M. cochinchinensis (Chiche et al. (2004) Curr. Protein Pept. Sci. 5(5):341-349), is a linearized version of MCoTI-I/II at loop 6. This should allow the linearized cyclotides to fold correctly.

TABLE 1
Human Proteins for Interacting Domains

| Protein A | Protein B | Function | Known modifications |
|---|---|---|---|
| p53 (1-30) | mdm2 | Targets p53 for | Phosphorylation of p53 |
| | (1-102) | degradation | S15 by ATM inhibts |
| | | | interaction |
| mdm2 | mdm2 | Dimerizes mdm2, | Mutations affecting |
| (438-479) | (438-479) | required for E3 | Zn-finger destroy E3 |
| RING | RING | activity | activity |
| domain | domain | | |
| BARD-1 | BRCA-1 | Hetero-dimerization, | Mutations affecting |
| (50-87) | (24-65) | required for tumor | BRCA-1 RING domain |
| RING | RING | suppressor activity | associated with different |
| domain | domain | of BRCA1 | types of cancer (breast |
| | | | and ovarian) |
| BRCA1 | Linearized | Required for | Mutations affecting |
| (1642-1736) | cyclotide | interaction with | BRCA-1 BRCT domain |
| BRCT-1 | library | multiple proteins | associated with different |
| domain | | | types of cancer |
| BRCA1 | | Required for | (breast and ovarian) |
| (1756-1855) | | interaction with | |
| BRCT-2 | | multiple proteins | |
| domain | | | |

TABLE 2
Different Orthogonal and Tightly Controlled Inducible Bacterial Expression
Systems That Can Be Used for the Sequential Co-expression of the Library
and Screening Plasmids in E. coli Cells.

| | Library plasmid | | Screening plasmid | | | |
|---|---|---|---|---|---|---|
| Promoter | TetA | araBAD | T7/lac | | | |
| Antibiotic | Amp | Amp/Kan | Cam | Strep | Kan | |
| resistance | | | | | | |
| Origin of | ColE1 | ColE1 | p15a | CloDF13 | pRSF1030 | ColA |
| replication | | | | | | |
| Plasmid | pASK75 | pBAD | pACYC | pCDF | pRSF Duet | pCOLA |
| family | | | Duet | Duet | | Duet |
| Copy | about 40 | about 40 | 10-12 | 20-40 | >100 | 20-40 |
| number | | | | | | |

### Lethality-Based In Vivo Screening Approach

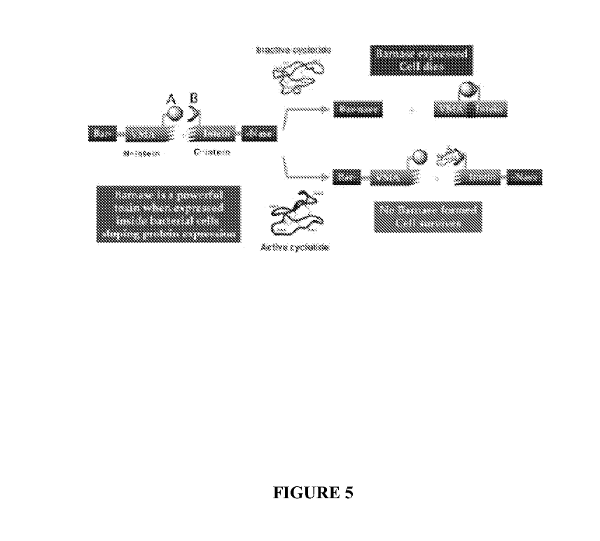

Incorporating the principles of Darwinian selection a lethality-based in-vivo screening method aimed at identifying cyclotide library member(s) capable of inhibiting the interaction between a particular set of proteins has been designed (FIG. 5).

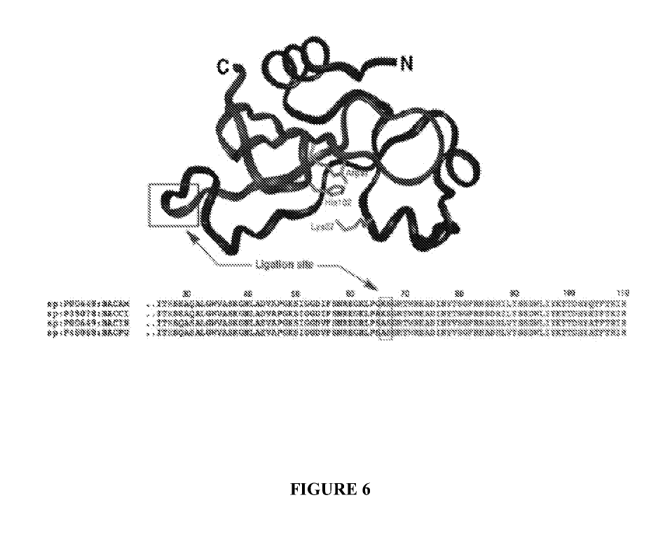

By engineering a reporter system based on conditional protein splicing (CPS) (Mootz et al. (2003) J. Am. Chem. Soc. 125(35):10561-10569), cells survival will be dependent on the presence of an active library member that blocks the interaction between the two interacting proteins or protein domains. The principle is based on the reconstitution of Barnase using protein conditional splicing. Bacillus amyloliquefaciens Barnase is an extensively well-characterized N1/T1 ribonuclease that efficiently triggers cell death when it is expressed and properly folded inside cells. In this approach a naturally occurring intein (VMA intein) is artificially split into two fragments (N-intein and C-intein). In contrast with naturally split inteins, these two fragments do not have affinity for each other and in absence of any other interaction they remain inactive, i.e. unable to produce protein splicing. However, when two interacting proteins are fused to the corresponding intein fragments (FIG. 5), these two fragments are brought into close proximity and undergo correct folding. This intermolecular folding event induces protein splicing and therefore formation of Barnase, which then triggers cellular death. This enzyme is extremely well conserved between different species of Bacillus as shown in FIG. 6.

The split location has been chosen away from the active site and it is located in an exposed and variable loop (FIG. 6). In order to facilitate conditional protein splicing, residue Ser67 can be mutated to Cys. It is anticipated that this conservative mutation will not modify the activity of the resulting enzyme. This screening system will produce the full-length cytotoxic Barnase only if the split intein becomes active. As before, to facilitate the interaction between the two interacting proteins or protein domains and prevent any steric hindrance that will impede efficient protein trans-splicig, appropriate flexible polypeptide linkers (i.e. [Gly-Gly-Ser]n, wherein n is an integer of 1 or greater than 1) will be also added at the junctions between the interacting proteins and the corresponding half-intein constructs. Conditional protein trans-splicing can be also tested in vivo by co-expressing the best trans-splicing set of proteins using the pET-DUET system, commercially available from Novagen. In this case, the residue His102 in the C-terminal Barnase fragment can be mutated to Gln. This mutation is known to inactivate the enzymatic activity of Barnase and therefore it will allow estimate the efficiency of trans-splicing in vivo without triggering cell death.

### Sequential Expression of the Cyclotide-Based Library and Cell-Based Screening System

This aspect of the invention involves the creation of two bacterial expression plasmids, the library and the screening plasmids. Both plasmids contain different origins of replication, also termed “discrete origins of replication” (such as ColE1, p15A or pRSF1030, among others, see Table 2) and different inducible bacterial promoters, also termed “discrete promoters” (such as arabinose, tetracycline or T7 promoters among others). This permits the tight control the level of expression of the cyclic peptides and the screening constructs (either by FRET or Darwinian selection) and prevents replication dominance of one plasmid. This also allows both plasmids to be stable in the same cell. This approach involves first the expression of the cyclotide-based peptide library followed by the expression of the corresponding screening system. The generic vectors pASK75 or pBAD (Table 2) can be for the inducible expression of the plasmid library. The components of the screening system (either by FRET or lethality-based) can be expressed using the DUET expression system. This family of vectors contains two multi-cloning sites (MCS), each of which is preceded by a T7 promoter/lac operator and a ribosome binding site. The vectors of this family carry all different replicons as well as drug resistance genes (Table 2), which make them ideal candidates for the co-expression of the two components of the screening system. The cell is first loaded with the potential inhibitor or test agent. Expression of the in-cell reporter associated proteins is then induced. This allows the cyclization and folding of the cyclotide inside the bacterial cytoplasm before the screening can take place.

### Screening and Selection

In the case of the FRET-based reporter the cells are screened and selected by using FACS. Using the CyPet and YPet FRET couple, the FRET-on state is around 5 to 10 times more intense than the FRET-off state. Kimura et al. (2007) Anal. Biochem. 369(1):60-70. This allows the easy separation of positive clones by FACS. The process will require a few iterations to enrich the original library in cyclotide sequences that inhibit a particular target interaction. In the lethality-based screening approach, only cells able to inhibit the interaction that produces the Barnase toxin will survive. In this case, the screening and selection process are performed by culturing and plating under conditions that induce expression of the screening proteins. Discrimination between false positives and real positives are accomplished by activating only the expression of the screening plasmid. Under these conditions, where no cyclic inhibitors are induced, only the false positives are able to provide signal, i.e. FRET-off or survival.

### Targeted Human Proteins

A set of proteins and protein domains involved in tumor cell proliferation and suppression are used. These include p53, MDM2, MDMX and BRCA1. The MDM2 protein, which was originally identified as an oncogene, has been shown to be up-regulated in many human breast tumors and carcinomas, soft tissue sarcomas, and other cancers. The main cellular function of MDM2 is to regulate p53 levels. Principally, MDM2 promotes p53 degradation through an ubiquitin-dependent pathway. The tumor suppressor p53 is a potent transcription factor, which is activated following cellular stress and regulates multiple downstream genes involved in cell cycle and apoptosis hence playing a pivotal role in protection from cancer. The methods of this invention identifies cyclotides able to bind to the p53 binding domain of MDM2 as well as able to inhibit the interaction between this domain and its target the N-terminal fragment of p53. Other cancer relevant targets for our approach will be the RING domains from MDM2/MDMx and BRCA1-BARD1 and the BRCT domains of BRCA1. All these domains are involved in the formation of homo- and/or heterodimers required for biological function, and are also important biomarkers as well as therapeutic targets in cancer.

### Site-Specific Immobilization of Cyclotide-Based Antagonists

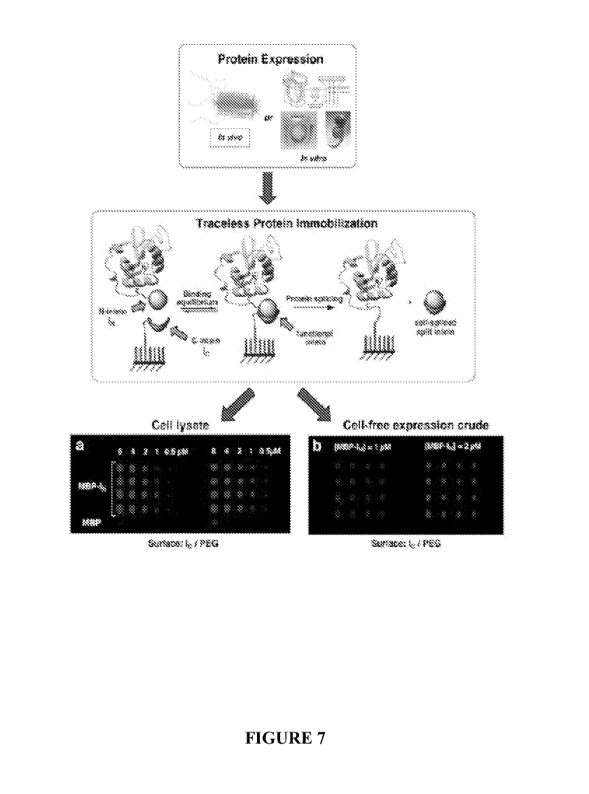

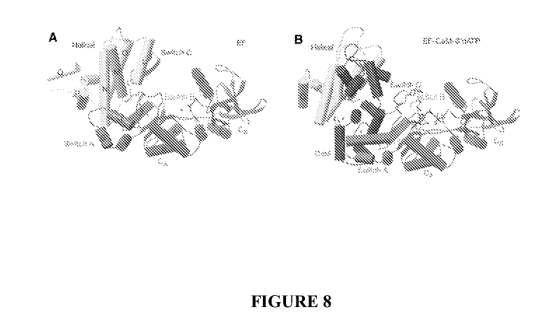

Although numerous approaches have been described in the literature for the immobilization of biomolecules onto solid-supports, for example using chemoselective reactions, affinity tags, and capture ligand approaches; most of them require purification and concentration of the biomolecule to be immobilized and/or require the use of large capture proteins that remain attached to the surface once the immobilization process is done. In some cases, the presence of such a large linkers could give rise to problems, especially in those applications where the immobilized proteins will be involved in studying protein-protein interactions with complex protein mixtures. To address this problem, a new traceless capture ligand approach for the selective immobilization of proteins to surfaces based on the protein trans-splicing process has been developed (FIG. 7). Kwon et al. (2006) Angew. Chem. Int. Ed. 45(11):1726-1729.

For this invention, protein trans-splicing is used for the rapid and selective immobilization and traceless immobilization of linearized cyclotides onto solid-supports. Kwon et al. (2006) Angew. Chem. Int. Ed. 45(11):1726-1729. Protein immobilization using protein trans-splicing is highly specific and efficient. It allows the use of protein mixtures and eliminates the need for the purification and/or re-concentration of the proteins prior to the immobilization step. The required minimum protein concentration for efficient immobilization has been estimated to be sub-micromolar. Kwon et al. (2006) Angew. Chem. Int. Ed. 45(11):1726-1729. More importantly, once the protein is immobilized to the surface, both intein fragments are spliced out into solution, providing a completely traceless method of attachment (FIG. 7), in contrast with other ligand-capture approaches. All these features allow this methodology to be easily interfaced with cell-free protein expression systems with rapid access to the high-throughput production of protein chips capture reagents. Kwon et al. (2006) Angew. Chem. Int. Ed. 45(11):1726-1729. Initial studies are carried out using the cyclotide MCoTI-II that binds Trypsin with high affinity. Immobilization trials are performed using the DnaE N-intein-MCoTI-II construct produced by standard bacterial expression or cell-free expression systems. Kwon et al. (2006) Angew. Chem. Int. Ed. 45(11):1726-1729. Evaluation of the cyclotide-based microarrays are done using fluorescence (by tagging the different proteins to be captured) or mass spectrometry (MALDI-TOF).

### Experiment No. 2

### A Cell-Based Approach for the Rapid Biosynthesis/Screening of Cyclic Peptide Libraries Against Bacterial Toxins

This aspect provides a new combinatorial approach for the biosynthesis and screening of cyclic peptides inside living cells. These novel approaches are useful for finding biologically relevant molecules, e.g., those able to inhibit the cytotoxicity of Anthrax Edema Factor. Key to this ‘living combinatorial’ approach is the use of a living cell as a micro-chemical factory for both synthesis and screening of potential inhibitors for a given molecular recognition event. The great advantage of this approach is that all the processes (i.e. biosynthesis of the library and screening) happen in the cell and therefore no in vitro screening is required. This considerably speeds up the whole process. Furthermore, because the screening process is taking place in a complex media composed by thousand of proteins (i.e. inside the cytoplasma's cell) it is expected that only members of the library with high specificity for the target will be selected. This will minimize the selection of universal binders, a real problem when in vitro screening methods are employed.

In summary, the development of this novel approach introduces a generic technology for fast and efficient identification of high-affinity ligands that can be used as effective countermeasures to biological threats.

The exposure of U.S. postal workers to Bacillus anthracis, the pathogen causing Anthrax, in 2001 revealed a gap in the nation's overall preparedness against bioterrorism. These incidents underscored an urgent need to prevent, rapidly diagnose, and treat disease by developing new drugs that could be able to rapidly treat known or more importantly new pathogenic bacterial and viral strains.

B. anthracis infections, for example, are difficult to treat because flu-like symptoms appear only after the bacteria have multiplied inside the human host and started to produce the corresponding bacterial toxins that eventually cause death. If classical antibiotics are applied at this stage, the infection can be still lethal because the accumulation of the corresponding bacterial toxins. Therefore an effective therapeutic approach should include simultaneous treatment with classical antibiotics, which block bacterial growth, and neutralization of the corresponding Anthrax toxins with specific antitoxins. Therefore the development of new methods for fast and efficient synthesis, and screening of new drug-like molecules with a high affinity against toxins, should be one of the top priorities in the fight against bioterrorism. These molecules could be used as specific antidotes but also, when attached to the appropriate platform, as reliable biosensors.

In order to be useful, these new methodologies should be fast—allowing high throughput analysis—and efficient, i.e. provide specific binders in a short period of time. The available methods for solving this daunting problem are either based on rational or combinatorial approaches. The rational approach uses the molecular structure of the target to be knocked out, and then using docking software is able to find potential binders within a virtual library of small organic molecules. Despite the big advances in computing technology, however, this is still a slow process yielding around one ligand per year. Furthermore, most of the docking software available is based on static docking programs which reduces considerably the efficiency of this technique. Arkin et al. (2003) Proc. Natl. Acad. Sci. USA 100(4):1603-1608. Combinatorial approaches, on the other hand, use a random approach to generate as many compounds as possible with the hope that some of the members in these huge mixtures of compounds (also called libraries) will have activity against the molecular target (this is a common approach in nature, active antibodies are created and selected in that way). Combinatorial libraries can be generated using chemical (Lam et al. (1997) Chem. Rev. 97:411-448) or biological (Clackson et al. (1991) Nature 352(6336):624-628) tools. Chemical libraries are usually generated in solid-phase and consequently the libraries are limited to contain 10⁶ different compounds as maximum. Lam et al. (1997) Chem. Rev. 97:411-448. Biological libraries can reach up to 10⁹ members per library (as in phage display technology), however, they are mostly limited to the generation of peptide/proteins libraries only. In both cases, however, the main limitation is the screening process, which is carried in vitro and is time consuming.

As solution this invention provides a totally new and revolutionary approach, a living combinatorial approach. This approach uses living cells for the generation of libraries of biomolecules, which are then screened inside the cell for activity (see FIG. 1). The advantage of this method is that both processes—biosynthesis of the library and screening—occur inside the cell, eliminating the need for in vitro screening. This approach considerably speed up the process. Furthermore, because screening takes place inside the cytoplasm's cell, where thousands of proteins are present, only those members of the library with high specificity for the biomolecular target will be selected. In one aspect, this approach is used for finding specific inhibitors against the Anthrax Edema Factor (EF) as proof of principle.

### Anthrax, a Perfect Biological Weapon System

Anthrax is an infectious disease caused by the Gram-positive and spore-forming bacterium Bacillus anthracis. Dixon et al. (1999) N. Engl J. Med. 341(11):815-826. The anthrax spore is an ideal and powerful bioweapon based on its hardiness and the effective morbidity and mortality of the inhalational form of anthrax.

This deadly form of anthrax is produced when anthrax spores are inhalated and then taken by alveolar macrophages to the lymph nodes where germination occurs. The bacteria then replicate to very high numbers, ultimately leading to the rapid death of the host. Dixon et al. (1999) N. Engl. J. Med. 341(11):815-826; Mock et al. (2001) Annu Rev. Microbiol. 55:647-671. The high pathogenicity of anthrax is mostly due to the rapid bacterial growth combined with the secretion of three powerful exotoxin components: edema factor (EF), lethal factor (LF) and protective antigen (PA). EF is a calcium and calmodulin-dependent adenylylate cyclase (AC) that converts cellular ATP into cyclic AMP (cAMP). Leppla (1982) Proc. Natl. Acad. Sci. USA 79(10):3162-3166. LF is a Zn²⁺ dependent metalloprotease (Klimpel et al. (1994) Mol. Microbiol. 13(6):1093-1100) that cleaves and inactivates mitogen-activated protein kinase kinases (MAPKKs). Montecucco et al. (2004) Trends Biochem. Sci. 29(6):282-285. PA binds to a cell surface anthrax toxin receptor (ATR/TEM-8 or CMG-2) (Bradley et al. (2001) Nature 414(6860):225-229; Scobie et al. (2003) Proc. Natl. Acad. Sci. USA 100(9):5170-5174; Santelli et al. (2004) Nature 430(7002):905:8) where is activated by proteolytic cleavage by furin-like proteases. Gordon et al. (1995) Infect. Immun. 63(1):82-87. This step enables the formation of an heptameric pore (Milne et al. (1994) J. Biol. Chem. 269(32):20607-20612) that allows cellular entry of LF and EF. Once inside the cell, LF and EF cause extensive cellular damage to the host cell defense system. LF can induce apoptosis in macrophages and endothelial cells (Park et al. (2002) Science 297(5589):2048-51; Kirby (2004) Infect. Immun. 72(1):430:9) and impairment of dentritic cells. Agrawal et al. (2003) Nature 424(6946):329-334. EF can impair host innate and adaptive immunity by altering the phagocytic activity of macrophages, cytokine production by monocytes and macrophages, and antigen presentation of T cells. Paccani et al. (2005) J. Exp. Med. 201(3):325-331. The disruption of the EF gene has been shown to significantly reduce the survival ability and lethality of B. anthracis in vivo. Brossier et al. (2000) Infect. Immun. 68(4):1781-1786.

Non-toxigenic strains of B. anthracis, on the other hand, are poorly pathogenic indicating the importance that anthrax toxins play in all stages of the pathogenesis of the disease, from the very beginning of infection to death. As mentioned before, anthrax is particularly dangerous because it is asymptomatic until the bacterium reaches the blood. Dixon et al. (1999) N. Engl. J. Med. 341(11):815-826; Mock et al. (2001) Annu Rev. Microbiol. 55:647-671. Once in the blood system, B. anthracis multiplies so rapidly that it is unlikely that antibiotic therapy could prevent death. In this context, it is essential to develop anti-toxin therapeutics to be used for preventive and/or alone or in combination with antibiotics.

### Anthrax EF, an Attractive Target for Developing Bacterial Toxin Inhibitors

Anthrax EF is a class II adenylate cyclase. This family of cyclases are only found in bacteria and also include other toxins secreted by pathogenic bacteria, such as cyclase CyaA from Bordetella pertussis, the causative agent of whooping cough, ExoY from Pseudomonas aeruginosa, bacteria responsible for various nocosomial infections, and the adenylate cyclase from Yersinia pestis, the causing agent of the plague. These adenylate cyclase toxins enter the eukaryotic host cells and become activated by binding to cellular factors that trigger the intracellular synthesis of cAMP. The immune effector cells appear to be the primary target of these type of toxins. By accumulating cAMP in the immune effector cells, these toxins poison the immune system and thus facilitate the survival of the bacteria in the host.

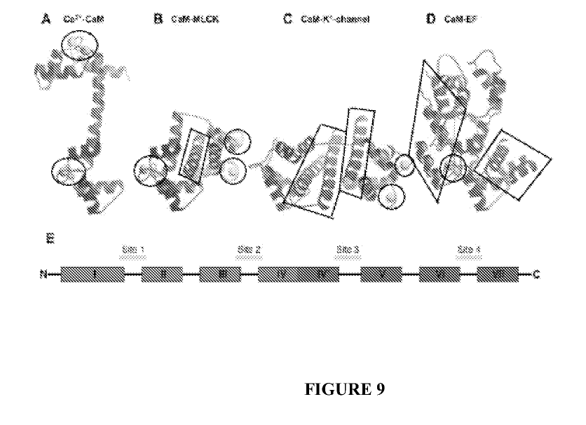

EF forms a tight complex (K_{d}=20 nM) (Drum et al. (2000) J. Biol. Chem. 275(46):36334-36340) with intracellular CaM once is delivered inside the cell. This EF-CaM complex catalizes the conversion of intracellular ATP to cAMP around 1000 times faster than EF alone. Drum et al. (2002) Nature 415(6870):396-342. The structural comparison of the catalytic domain of EF with Class III cyclases, which are the only cyclases found in eukaryotic cells, reveals no structural similarity between these two families of enzymes. Moreover, the crystal structure of the EF-CaM complex (FIG. 8) has revealed that this protein-protein interaction is quite distinct from other CaM binding proteins (FIG. 9). Drum et al. (2002) Nature 415(6870):396-342; Hoeflich et al. (2002) Cell 108(6):739-742. Structural analysis of several complexes of CaM with CaM-binding proteins reveals that the central α-helix of CaM (helix IV-IV′, FIG. 9) is kinked and wrapped around the α-helical CaM binding domains (FIG. 9). However, in the structure of EF-CaM (FIGS. 8 and 9), the central α-helix of CaM adopts a more extended conformation surrounded by two domains of EF (FIG. 8). All these facts make anthrax EF an attractive target for the development of bacterial toxin inhibitors. Lee et al. (2004) Chem Biol 11(8):1139-1146.

### Cyclic Peptides Libraries as a Source of Highly Specific Toxin Inhibitors

A significant number of natural products with wide range of pharmacological activities derive from cyclic polypeptides. In fact, peptide cyclization is widely used in medicinal chemistry to improve the biochemical and biophysical properties of peptide-based drug candidates. Hruby et al. (1990) J. Biochem. 268:249-262; Rizo et al. (1992) Ann. Rev. Biochem. 61:387-418. Cyclization rigidifies the polypeptide backbone structure, thereby minimizing the entropic cost of receptor binding and also improving the stability of the topologically constrained polypeptide. Among the different approaches used to cyclize polypeptides, backbone or head-to-tail cyclization remains one of the most extensively used to introduce structural constraints into biologically active peptides. In FIG. 2, the primary and tertiary structures of two naturally occurring cyclotides, MCoTI-II (left identified) and Kalata B1 (right) are shown. The disulfide bonds forming the Cys-knot fold are shown.

Among the different cyclic peptides available from natural sources, this invention uses the cyclotide scaffold as template for creating molecular libraries. Cyclotides are a newly emerging family of large backbone cyclic polypeptides (≈30 residues long) characterized by a disulfide-stabilized core (3 disulfide bonds) with an unusual knotted structure (FIG. 3). Craik et al. (1999) J. Mol. Biol. 294(5):1327-1336; Trabi et al. (2002) Trends Biochem. Sci. 27(3):132-138; Craik et al. (2004) Curr. Protein Pept. Sci. 5(5):297-315; Goransson et al. (2004) Curr. Protein Pept. Sci. 5(5):317-329; Vogel et al. (2005) Structure (Camb) 13(5):688-690. In contrast to other cyclic polypeptides, cyclotides have a well-defined three-dimensional structure. Therefore, despite their small size, they can be considered mini-proteins. The unique cyclic-backbone topology and knotted arrangement of 3 disulfide bonds endow cyclotides with exceptional stability and resistance to chemical, enzymatic and thermal degradation. Gorannson et al. (2003) J. Biol. Chem. 278(48):48188-48196; Cograve et al. (2004) Biochemistry 43(20):5965-5975. Furthermore, their well-defined structures have been associated with a range of biological functions such as uterotonic activity, inhibition of trypsin and neurotension binding, cytotoxicity, anti-HIV, antimicrobial, and insecticidal activity. Gran (1973) Acta. Pharmacol. Toxicol. (Copenh) 33(5):400-408; Gran (1973) Lloydia 36(2):174-178; Witherup et al. (1994) J. Nat. Prod. 57(12):1619-1625; Gustafson et al. Curr. Protein Pept. Sci. 5(5):331-340; Tam et al. (1999) Proc Natl Acad Sci USA 96(16):8913-8918; Jennings et al. (2001) Proc. Natl. Acad. Sci. USA 98(19):10614-10619; Felizmenio-Quimio et al. (2001) J. Biol. Chem. 276(25):22875-22882. Together, these characteristics suggest that cyclotides are ideal molecular scaffolds for the development of stable protein inhibitors. Craik et al. (2004) Curr. Protein Pept. Sci. 5(5):297-315; Craik (2002) Curr. Opin. Drug Discov. Devel. 5(2):251-260.

Applicants have been recently able to biosynthesize several cyclotide-based libraries using protein splicing tools in vitro under conditions mimicking those found in the cytoplasm of living cells. Kimura et al. (2006) Angew Chem. Int. Ed. Engl. 45(6):973-976.

### Biosysnthesis of Cylotide-Based Peptides Libraries

Despite the fact that the chemical synthesis of cyclic peptides has been well explored and a number different approaches involving solid-phase or liquid-phase exist (Camarero (1997) J. Chem. Soc. Chem. Comm. 1997:1369-1370; Zhang et al. (1997) J. Am. Chem. Soc. 119:2363-2370; Camarero et al. (1998) Angew. Chem. Int. Ed. 37(3):347-349; Shao et al. (1998) Tetrahedron Lett. 39(23):3911-3914; Camarero, J. A. et al. (1998) 51:303-316), recent developments in the fields of molecular biology and protein engineering have now made possible the biosynthesis of cyclic peptides. This progress has been made mainly in two areas, non-ribosomal peptide synthesis (Trauger et al. (2000) Nature 407(6801):215-218; Kohli et al. (2002) Nature 428(6898):658-661; Walsh (2004) Science 303(5665):1805-1810) and expressed protein ligation/protein trans-splicing. Camarera et al. (1999) J. Am. Chem. Soc. 121:5597-5598; Scott et al. (1999) Proc. Natl. Acad. Sci. USA 96(24):13638-13643; Evans et al. (1999) J. Biol. Chem. 274(26):18359-18381; Camarero et al. (2001) Bioorg. Med. Chem. 9(9):2479-2484; Iwai et al. (2001) J. Biol. Chem. 276(19):1654; Abel-Santos et al. (2003) Methods Mol. Biol. 205:281-294. The former strategy involves the use of genetically engineered non-ribosomal peptide synthetases and is reminiscent of more established technologies that yield novel polyketides. The later strategy relies on the heterologous expression of recombinant proteins fused to modified intein protein splicing/trans-splicing units. Noren et al. (2000) Angew. Chem. Int. Ed. 39(3):451-456.

The biosynthesis of cyclic polypeptides offers many advantages over purely synthetic methods. Using the tools of molecular biology, large combinatorial libraries of cyclic peptides, may be generated and screened in vivo. A typical chemical synthesis may generate 10⁴ different molecules. It is not uncommon for a recombinant library to contain as many as 10⁹ members. The molecular diversity generated by this approach is analogous to phage-display technology. Moreover, this approach takes advantage of the enhanced pharmacological properties of backbone-cyclized peptides as opposed to linear peptides or disulfide-stabilized polypeptides. Also, the approach differs from phage-display in that the backbone-cyclized polypeptides are not fused to or displayed by any viral particle or protein, but remain on the inside of the living cell where they can be further screened for biological activity. In contrast to phage display, where the screening takes place in vitro, screening that takes place in the cytoplasm offers the advantages conferred by a native physiological environment where diverse biochemical events may be examined. The complex cellular cytoplasm provides the appropriate environment for the rapid selection of highly specific able to attenuate or inhibit particular molecular recognition events.

### In Vivo Screening Using Conditional Protein Trans-Splicing

The biosynthesized cyclotide-based libraries are screened in vivo using a generic Darwinian approach. Key to this approach is the use of conditional protein splicing (CPS). Mootz et al. (2003) J. Am. Chem. Soc. 125(35):10561-9; Mootz et al. (2002) J. Am. Chem. Soc. 124(31):9044-9045. The principle is based on the reconstitution of a toxin using protein conditional splicing. In this approach a naturally occurring intein is artificially split into two fragments (N-intein and C-intein). Mootz et al. (2002) J. Am. Chem. Soc. 124(31):9044-9045. In contrast with naturally split inteins, these two fragments do not have affinity for each other and in absence of any other interaction they remain inactive, i.e. unable to produce protein splicing.

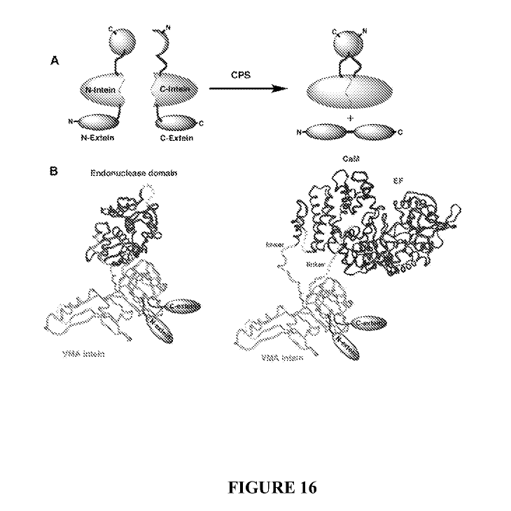

However, when two interacting proteins, such as EF and CaM are fused to the corresponding intein fragments (FIG. 16), these two fragments are brought into close proximity and undergo correct folding. This intermolecular folding event induces protein splicing and therefore formation of the toxin, which then triggers cellular death. In the presence of an effective inhibitor for the molecular interaction between EF and CaM, the protein splicing-mediated generation of the toxin will be stopped thus enabling the corresponding cells to survive. Hence this screening system will provide an effective way to select for those cells able to produce affective inhibitors against the interaction between EF and CaM.

The ability to create cyclic polypeptides in vivo opens up the possibility of generating large libraries of cyclic polypeptides. Using the tools of molecular biology, genetically encoded libraries of cyclic polypeptides containing billions of members can be readily generated. This tremendous molecular diversity forms the basis for selection strategies that model natural evolutionary processes. Also, since the cyclic polypeptides are generated inside living cells, these libraries can be directly screened for their ability to inhibit cellular processes or any particular protein interaction. In vivo screening allows the rapid selection of potential leads in complex environments such as the cellular cytoplasm, which assures that only highly specific inhibitors will be selected thus minimizing the selection of non-specific binders.

Cyclic polypeptides are relatively more stable and more resistant to cellular catabolism than linear polypeptides or disulfide-based cyclic polypeptides and therefore are ideal candidates for developing molecular inhibitors against biological toxins. Naturally occurring cyclic peptides often exhibit diverse therapeutic activities ranging from immuno-suppression to antimicrobial activity. The cyclotide scaffold, in particular, is an extremely interesting novel class of circular, disulfide-rich peptides that display a broad range of bioactivities and have exceptionally high stability to extreme conditions. Their extreme physical properties, which include resistance to thermal and enzymatic degradation, can be attributed to their unique cyclic backbone and knotted arrangement of disulfide bonds. These exceptional characteristics make them ideal templates for drug design and discovery as well as for developing stable and specific antidotes against biological toxins.

This invention is useful, inter alia, for the production of highly specific cyclotide-based inhibitors against the anthrax EF protein as a proof of principle. Anthrax still represents a significant hazard and it is becoming clear that more options are necessary to protect Americans from the threat of an anthrax attack. An anthrax vaccine is available and effective, but it requires six injections over 18 months, plus yearly boosters to provide full immunity. The only other treatment available is antibiotics, which destroy the anthrax bacteria, but they are only effective when it is known that people have been exposed to anthrax. Furthermore, antibiotics are not effective anthrax toxin inhibitors once the bacterial toxins have been released into the bloodstream. Hence, the development of effective anthrax toxin inhibitors able to inactivate the toxins from the bloodstream even after symptoms appear may prove a crucial approach to save time for later-stage antibiotic treatment.

In summary, the development of this novel living combinatorial approach introduces a generic technology that combines chemistry and biology for fast and efficient identification of high-specific ligands to biological toxins. These ligands can be used as powerful antidotes against toxins. It is also anticipated that due to the high stability and high specificity of cylotide-based antidotes they could also be used as reliable and resistant biosensors when attached to the appropriate platforms, thus providing cheap and reliable detector systems that can be used to improve response capability to bioterrorist attacks on the military as well as on the civilian population.

An aspect of this invention is the use of a library of living cells. Each individual cell within this living library will be able to biosynthesize a unique cyclotide. This compound is then screened for activity against anthrax EF inside the cell (FIG. 1). Thus, in part, this invention provides: 1) a method for the biosynthesis of cyclotide-based libraries and 2) an in vivo screening process to select the cyclotide members of the library able to inhibit the interaction between EF and CaM.

### In Vivo Biosynthesis of Cyclic Peptide Libraries Based on the Cyclotide Scaffold

As noted above, cyclotides are a newly emerging family of large backbone cyclic polypeptides (about 30 residues long) characterized by a disulfide-stabilized core (3 disulfide bonds) with an unusual knotted structure. Trabi et al. (2002) Trends Biochem. Sci. 27(3):132-8; Craik et al (2004) Curr. Protein Pept. Sci. 5(5):297-315; Goransson et al. (2004) Curr. Protein Pept. Sci. 5(5):317-29; Vogel et al. (2005) Structure (Camb) 13(5):688-690. The core structural motif in cyclotides has been termed cyclic cystine knot (CCK) and is characterized by a cystine knot embedded into a circular backbone topology (see FIG. 3). Vogel et al. (2005) Structure (Camb) 13(5):688-690. The cystine knot involves two disulfide bonds, which form a ring that is penetrated by a third disulfide bond. The unique cyclic-backbone topology and knotted arrangement of 3 disulfide bonds endow cyclotides with exceptional stability and resistance to chemical, enzymatic and thermal degradation. Colgrave et al. (2004) Biochemistry 43(2):5965-5975. There are currently around 100 published sequences of cyclotides and their rate of discovery has been increasing over recent years. Ultimately the family may comprise thousands of members. Furthermore, cyclotides also show a diverse range of biological activities. Chen et al. (2005) J. Biol. Chem. 280(23):22395-405. All these features make the cyclotide framework an ideal peptide scaffold for finding highly stable and specific protein-protein inhibitors.

### In Vivo Biosynthesis Using Protein Splicing Tools

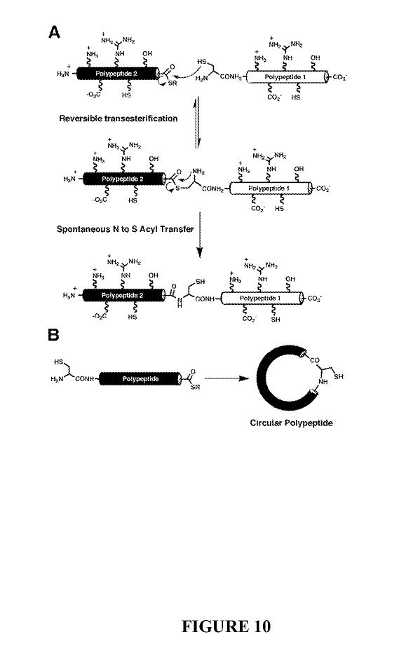

In vivo biosynthesis of cyclotide-based peptides is accomplished by using an intramolecular version of the Native Chemical Ligation (NCL) reaction. Camarero et al. (1997) J. Chem. Soc., Chem. Comm. 1997:1369-1370; Camarero et al. (1998) Angew. Chem. Int. Ed. 37(3):347-349; Camarero et al. (1999) J. Am. Chem. Soc. 121:5597-5598; Dawson et al. (1994) Science 266:776-779; Tam et al (1995) Proc. Natl. Acad. Sci. USA 92(26):12485-12489; Dawson et al. (2000) Annu. Rev. Biochem. 69:923-60. Native Chemical Ligation (NCL) is an exquisitely specific ligation reaction that has been extensively used for the total synthesis, semi-synthesis and engineering of different proteins. Dawson et al. (2000) Annu Rev. Biochem. 69:923-60; Evans et al. (1999) Biopolymers 51(5):333-42; Camarero et al. (1999) Current Protocols in Protein Science (18.4):1-21; Muir (2003) Annu Rev. Biochem. 72:249-289. In this reaction, two fully unprotected polypeptides, one containing a C-terminal α-thioester group and the other a N-terminal Cys residue, react chemoselectively under neutral aqueous conditions with the formation of a native peptide bond (see FIG. 10A). The initial step in this ligation involves the formation of a thioester-linked intermediate, which is generated by a trans-thioesterification reaction involving the α-thioester moiety of one fragment and the N-terminal Cys thiol group of the other fragment. This intermediate then spontaneously rearranges to produce a peptide bond at the ligation site. It is well established that when these two reactive groups, i.e. the C-terminal α-thioester group and the N-terminal Cys residue, are located in the same synthetic precursor, the chemical ligation proceeds in an intramolecular fashion thus resulting in the efficient formation of a circular polypeptide (see FIG. 10B). This reaction has been successfully employed for the chemical synthesis of cyclic peptides and small protein domains. Camarero et al. (1997) J. Chem. Soc. Chem. Comm. 1997:1369-1370; Camarero et al. (1998) Angew. Chem. Int. Ed. 37(3):347-349; Shao et al. (1998) Tetrahedron Lett. 39(23):3911-3914; Camarero et al. (1998) J. Pept. Res. 51:303-316.

Recent advances in protein engineering have made possible the introduction of the C-terminal α-thioester group and N-terminal Cys residue into recombinant proteins. These important developments make possible the use of NCL between synthetic and/or recombinant fragments. This new technology, called Expressed Protein Ligation (EPL), now allows access to a multitude of chemically engineered recombinant proteins including biosynthetic circular polypeptides. Muir (2003) Annu Rev. Biochem 72:249-289.

### Generation of Recombinant Polypeptide α-Thioesters

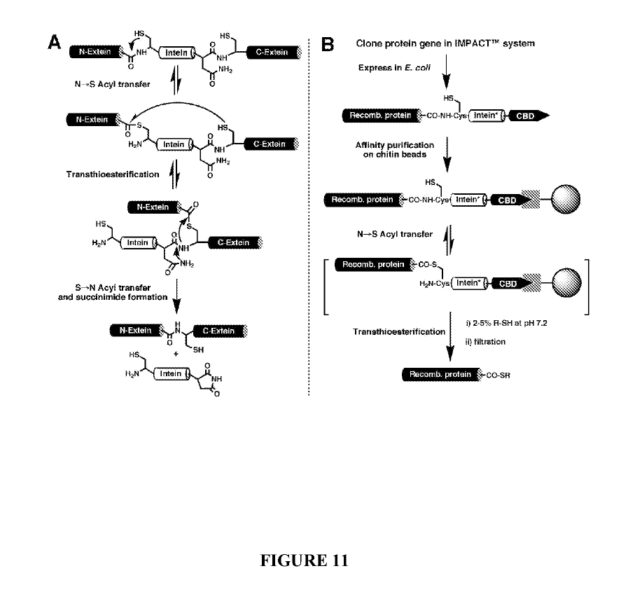

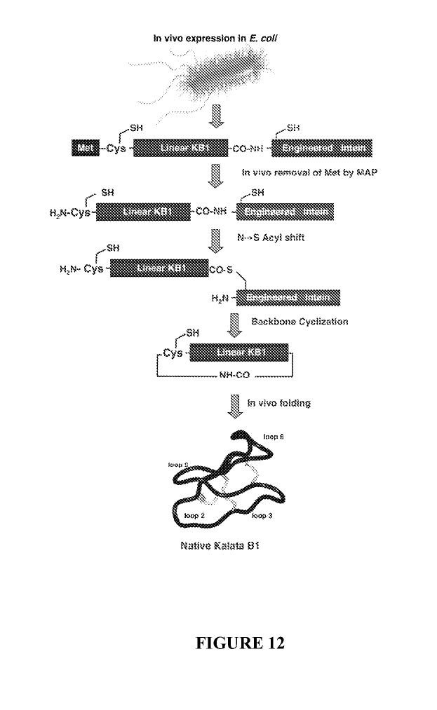

Recombinant protein α-thioesters can be obtained by using engineered inteins. Camarero et al. (1999) Current Protocols in Protein Science (18.4):1-21; Muir et al. (1998) Proc. Natl. Acad. Sci. USA 95(12):6705-10; Severinov et al. (1998) J. Biol. Chem. 273(26):16205-9; Evans et al. (1998) Protein Sci. 7:2256-2264. Inteins are self-processing domains which mediate the naturally occurring process called protein splicing. Xu et al. (1996) EMBO J. 15(19):5146-5153 (see FIG. 11).

Protein splicing is a cellular processing event that occurs post-translationally at the polypeptide level. In this multistep process an internal polypeptide fragment, called intein, is self-excised from a precursor protein and in the process ligates the flanking protein sequences (N- and C-exteins) to give a different protein. The current understanding of the mechanism is summarized in FIG. 11 and involves the formation of thioester/ester intermediates. Xu et al. (1996) EMBO J. 15(19):5146-5153. The first step in the splicing process involves an N→S or N→O acyl shift in which the N-extein is transferred to the thiol/alcohol group of the first residue of the intein. After the initial N→(S/O) acyl shift, a trans-esterification step occurs in which the N-extein is transferred to the side-chain of a second conserved Cys, Ser or Thr residue, this time located at the junction between the intein and the C-extein. The amide bond at this junction is then broken as a result of succinimide formation involving a conserved Asn residue within the intein. In the final step of the process, a peptide bond is formed between the N-extein and C-extein following an (S/O→N acyl shift (similar to the last step of Native Chemical Ligation, see FIG. 4A). Mutation of the conserved Asn residue within the intein to Ala blocks the splicing process in midstream thus resulting in the formation of an α-thioester linkage between N-extein and the intein. Xu et al. (1996) EMBO J. 15(19):5146-5153 (see FIG. 11B). This thioester bond can be cleaved using an appropriate thiol through a trans-thioesterification step to give the corresponding recombinant polypeptide α-thioester. The IMPACT expression system, commercially available from New England Biolabs (Chong et al. (1997) Gene 192(2):271-81; Chong et al. (1998) Nucleic Acids Res. 26(22):5109-15), allows the in vivo and in vitro generation of recombinant α-thioester proteins by making use of such modified inteins (see FIG. 11B). The pTXB and pTYB families of vectors, which contain modified inteins derived from the Gyrase and VMA inteins, respectively, can be used for this purpose.

### Generation of N-Terminal Cys-Containing Peptides

The introduction of N-terminal Cys residues into expressed proteins can be readily accomplished in vivo or in vitro by cleaving (by proteolysis or auto-proteolysis) the appropriate fusion proteins. The simplest way to generate a recombinant polypeptide containing a N-terminal Cys residue is to introduce a Cys downstream to the initiating Met residue. Once the translation step is completed, the endogeneous methionyl aminopeptidases (MAP) removes the Met residue, thereby generating in vivo a N-terminal Cys residue. Camarero et al. (2001) Bioorg. Med. Chem. 9(9):2479-84; Hirel et al. (1989) Proc. Natl. Acad. Sci. USA 86(21):8247-51; Dwyer et al. (2000) Chem. Biol. 7(4):263-74; Iwai et al. (1999) FEBS Lett. (459):166-172; Cotton et al. (1999) J. Am. Chem. Soc. 121(5):1100-1101. Other approaches involve the use of exogenous proteases. Verdine and co-workers added a Factor Xa recognition sequence immediately in front of the N-terminal Cys residue of the protein of interest. Erlandson et al. (1996) Chem. Biol. 3:981-991. After purification, the fusion protein was treated with the protease Factor Xa, which generated the corresponding N-terminal Cys protein. Tolbert and Wong have also showed that the cysteine protease from tobacco etch virus (TEV) can also be used for the same purpose. Tolbert et al. (2002) Angew. Chem. Int. Ed. Engl. 41:2171-2174. This protease is highly specific and it can be overexpressed in E. coli. Other proteases that cleave at the C-terminal side of their recognition site, like enterokinase and ubiquitin C-terminal hydrolase, could be also used for the generation of N-terminal Cys residues.

In Vivo Biosynthesis of Cyclotide Polypeptides in E. coli

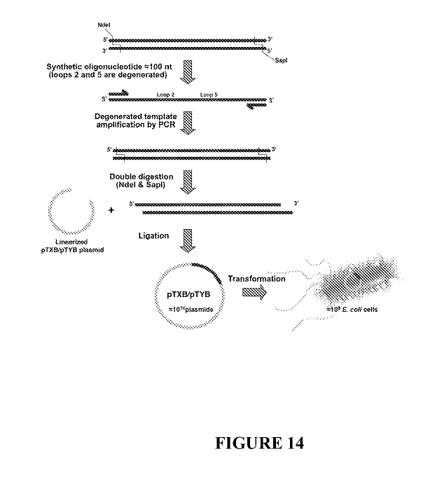

Described below is the in vivo biosynthesis of cyclotide polypeptides and is depicted in FIG. 14.

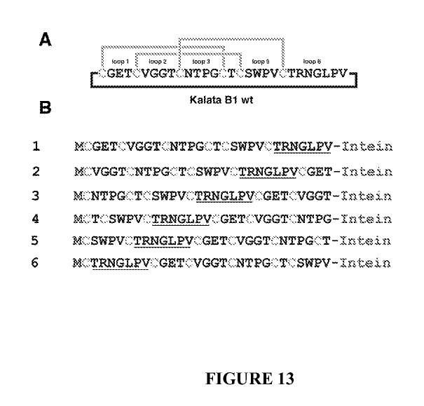

The very well-characterized cyclotides Kalata B1 (KB1) (Daly et al. (1999) Biochemistry 38(32):10606-14; Daly et al. (2000) Biol Chem 275(25):19068-75) and MCoTI-II (Felizmenio-Quimio et al. (2001) J Biol Chem 276(25):22875-82; Chen et al. (2005) J Biol Chem 280(23):22395-405) can be used. Briefly, several plasmids are constructed that encode the 6 different linear precursors of KB1 and MCoTI-II (FIG. 13) fused in frame at their C-termini to a modified intein (VMA or Gyrase). This allows the determination which N-terminal Cys provides the better ligation site for the cyclization reaction. It is important to remark that since cyclotides have 6 natural Cys residues there are in principle 6 potential ligation sites that can be used.

A Met residue can also genetically introduced at the N-terminus of the corresponding KB1 or MCoTI-II intein fusion protein (FIG. 13).

Once a particular cyclotide-intein fusion protein is expressed in E. coli cells, the first Met residue in the polypeptide sequence will be efficiently removed immediately by the endogenous Met aminopeptidase (MAP). Kimura et al. (2006) Angew Chem. Int. Ed. Engl. 45(6):973-6; Camarero et al. (2001) Bioorg. Med. Chem. 9(9):2479-84. This in vivo proteolytic event will unmask the required N-terminal Cys residue that can then react in an intramolecular fashion with the α-thioester generated by the engineered intein at the C-terminus of the linear cyclotide precursor. It is expected that the reduced cyclic cyclotide will then fold spontaneously in the cytoplasm to adopt the native cyclotide structure. Applicant has recently demonstrated that totally reduced cyclic KB1 can fold efficiently into its native form under conditions similar to those found in the cytoplasm. Kimura et al. (2006) Angew Chem. Int. Ed. Engl. 45(6):973-6.

Isolation and identification of in vivo biosynthesized cyclotides can be accomplished using a combination of reverse phase HPLC and mass spectrometry. MCoTI-II is a potent trypsin inhibitor with a tight affinity for trypsin (Kᵢ≈2 pM) and therefore immobilized trypsin beads could be also used for the isolation of native MCoTI-II from the cellular lysate. Structural characterization will be accomplished using standard 2D-homonuclear NMR techniques. Kimura et al. (2006) Angew. Chem. Int. Ed. Engl. 45(6):973-6.

Biosynthesis of KB1 and MCoTI-II can be explored using different E. coli strains. Special attention is given to engineered E. coli cell lines containing different mutations in the thioredoxin and glutathione reductase genes since they have been shown to facilitate disulfide bond formation in the cytoplasm of E. coli cells. Bessette et al. (1999) Proc. Natl. Acad. Sci. USA 96(24):13703-8. Some of these cell lines are commercially available from Novagen.

### In Vivo Biosynthesis of Cyclotide-Based Libraries

Cyclotide-based libraries can be created at the DNA level using double stranded DNA inserts with degenerated sequences for some of the different loops of the cyclotide scaffold (i.e. loops 2, 3 and/or 5, see FIG. 13).

Among the different strategies that have been developed to produce clonable degenerated DNA sequences (Sparks et al. (1996) Proc. Natl. Acad. Sci. USA 93(4):1540-1544; Kay et al. (1993) Gene 128(1):59-65; Christian et al. (1992) J. Mol. Biol. 227(3):711-8; Cwirla et al. (1990) Proc. Natl. Acad. Sci. USA 87(16):6378-82), the method described by Scott and Smith (Scott et al. (1990) Science 249(4967):386-90) (FIG. 14) can be used. This approach involves the generation of a double-stranded degenerated DNA by PCR. Briefly, a long degenerated synthetic oligonucleotide (which codes the whole cyclotide, ≈100 nt long) template is PCR amplified using 5′- and 3′-primers corresponding to the non degenerated flanking regions. The resulting double stranded degenerated DNA is double digested and a then ligated to a linearized intein expression vector (pTXB or pTYB family, FIG. 14) to produce a library of plasmids. These libraries are then transformed into electrocompetent E. coli cells to finally obtain a library of cells containing typically up to ≈10⁹ different clones (i.e. cyclotide sequences).

The degenerated synthetic oligonucleotide template can be synthesized using a NN(G/T) codon scheme for the degenerated regions. This scheme uses 32 codons to encode all 20 amino acids and encodes only 1 stop codon. Alternatively, the degenerated template can be also synthesized from mixtures of trinucleotide codons representing all 20 amino acids and no stop codons. Virnekas et al. (1994) Nucleic Acids Res. 22(25):5600-7.

It is anticipated that the first cyclotide-based libraries will be based on loops 2, 5 and/or 3 using the cyclotides KB1 and MCoTI-II as scaffolds. The complexity of these libraries will be around about 10⁶ and about 10⁹ members for combinations involving only one of the loops or two loops, respectively.

### A Lethality-Based In Vivo Screening of Cyclotide-Based Libraries

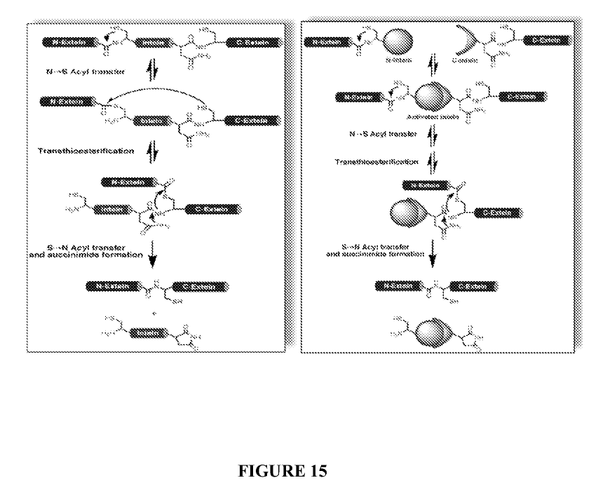

The screening process for finding which specific cyclotide peptides within the library are able to inhibit the EF-CaM interaction among others can be carried out in vivo by using a Darwinian selection scheme (see FIG. 1). Key to this approach is the use of conditional protein splicing (CPS). Mootz et al. (2003) J. Am. Chem. Soc. 125(35):10561-9; Mootz et al. (2002) J. Am. Chem. Soc. 124(31):9044-5; Ozawa et al. (2000) Anal. Chem. 72(21):5151-57. CPS allows to post-traslationally change the primary structure of a protein by using an engineered protein trans-splicing element that is dependent on the interaction of two different proteins (in this case EF and CaM). As mentioned before, protein splicing is an autocalytic process in which an intervening sequence, called intein, excises itself out of the precursor protein with concomitant linking of the flanking regions, termed exteins. In protein trans-splicing the intein domain is split in two pieces and the splicing event happens only after the two intein fragments are reconstituted (FIG. 15). In the CPS approach the cis-splicing VMA intein can be artificially split into N- and C-terminal halves, VMA^{N} and VMA^{C}, that have little or no affinity for each other. Mootz et al. (2003) J. Am. Chem. Soc. 125(35):10561-9; Ozawa et al. (2000) Anal. Chem. 72(21):5151-57; Kaihara et al. (2003) Anal Chem 75(16):4176-81. However, when two interacting proteins are fused to the C- and N-termini of the VMA^{N} and VMA^{C} halves, respectively, the binding event is able to bring together both intein fragments. This binding event triggers protein splicing and concomitant ligation of both N- and C-extein polypeptides (FIG. 15). Therefore, when the N- and C-extein polypeptides correspond to the complementary fragments of a split reporter protein, the fragmented reporter will remain inactive until the conditional protein splicing takes places. This approach has already been used to monitor protein-protein interactions in vivo using different protein reporters such as EGFP or luciferase among others. Ozawa et al. (2000) Anal. Chem. 72(21):5151-57; Kaihara et al. (2003) Anal. Chem. 75(16):4176-81; Kanno et al. (2006) Anal Chem 78(2):556-60.

One in vivo screening approach of this invention (see FIG. 11) uses the artificially split VMA intein system (VMA^{N}-intein (aa 1-184) and VMA^{C}-intein (aa 390-454). The Barnase N-terminal (aa 1-66) and C-terminal fragments (aa 67-110) are used as N- and C-extein polypeptides, respectively. Bacillus amyloliquefaciens Barnase is an extensively well-characterized N1/T1 ribonuclease that efficiently triggers cell death when it is expressed and properly folded inside cells. Bi et al. (2001) Gene 279(2):175-9. This enzyme is extremely well conserved between different species of Bacillus. The split location has been chosen away from the active site and it is located in an exposed and variable loop. In order to facilitate conditional protein splicing, residue Ser67 will be mutated to Cys. It is anticipated that this conservative mutation will not modify the activity of the resulting enzyme.

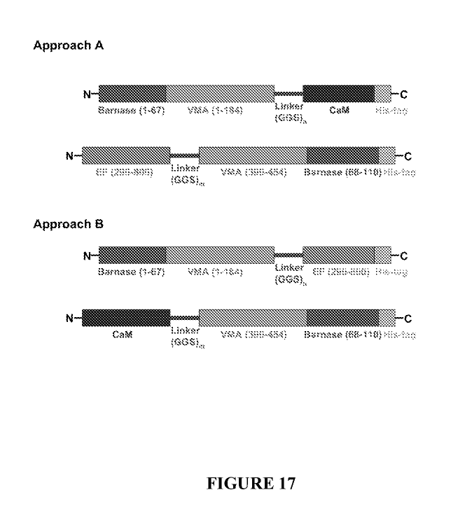

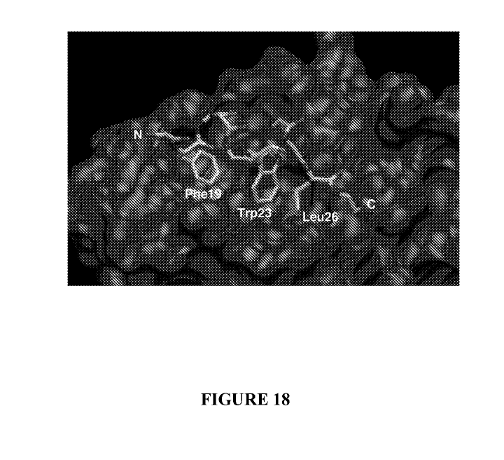

This expression system will produce the full-length cytotoxic Barnase only if the split intein becomes active. In order to screen in vivo potential antagonists for the interaction between EF and CaM, the adenylate cyclase domain of EF (aa 290-800) and full length CaM will be fused to the N-terminus of the VMA^{C}-intein-Barnase (aa 67-110) protein and to the C-terminus of the Barnase (aa 1-67)-VMA^{N}-intein construct, respectively. The other possibility, i.e. fusing the adenylate cyclase domain of EF and CaM to the C-terminus of the Barnase (aa 1-66)-VMA^{N} and to the N-terminus of the VMA^{C}-intein-Barnase (aa 67-110), respectively (FIG. 17), can also be used. Furthermore, in order to facilitate the interaction between EF and CaM and prevent any steric hindrance that will impede efficient protein trans-splicig, appropriate flexible polypeptide linkers (i.e. [GGS]ₙ) will be also added at the junctions between the interacting proteins (i.e. EF and CaM) and the corresponding half-intein constructs (see FIG. 17). These two sets of proteins can be expressed using a pET bacterial expression system and characterized first in vitro for their ability to trans-splice and produce active Barnase. These preliminary in vitro experiments will provide the best position (i.e. N-versus C-terminus) for both interacting proteins as well as the optimal linker length. Conditional protein trans-splicing will be also tested in vivo by co-expressing the best trans-splicing set of proteins using the pET-DUET system, commercially available from Novagen. In this case, the residue His102 in the C-terminal Barnase fragment will be mutated to Gln. This mutation is known to inactivate the enzymatic activity of Barnase and therefore it will allow to estimate the efficiency of trans-splicing in vivo without triggering cell death.

In summary, in vivo co-expression of this CPS system inside cells will provide a novel in vivo Darwinian screening approach for finding antagonists to the EF-CaM interaction. As shown in FIG. 1, only those cells being able to biosynthesize high affinity antagonists will be able to prevent the formation of Barnase and therefore survive.

Combine Biosynthesis of Cyclotide-Based Peptide Libraries with In Vivo Darwinian Screening for Rapid Detection of EF-CaM Antagonists

One embodiment provides a combination of both approaches, i.e. biosynthesis of cyclotide-based libraries with in vivo screening methods using E. coli cells. This involves the creation of two plasmids, the library and the screening plasmids. The library plasmid can be in fact a library of plasmids (in excess of 10⁹ members). All the plasmids of this library can contain a variable DNA sequence codifying for the different cyclotide peptides that can be present on the library. This variable sequence will be fused to the appropriate engineered intein element in order to allow its cyclization once expressed inside the cell. The screening plasmid can contain the two VMA split intein fusion proteins containing the Barnase fragments as well as the EF-CaM interacting polypeptide domains. Both plasmids will contain different origin of replication (such as ColE1, p15A or pRSF1030, among others) and different inducible bacterial promoters (such as arabinose, tetracycline or T7 promoters among others). This will permit to tightly control the level of expression of the cyclic peptides and the split VMA intein fusion proteins involved in the screening process.

This approach involves first the induced expression of the cyclotide-based peptide library followed by the expression of the Darwinian-based screening system. Under these conditions, only those cells being able to survive will contain potential positives, i.e. cyclotide peptides able to inhibit the formation of the EF-CaM protein complex. Discrimination between false and real positives will be accomplished by activating only the expression of the screening plasmid. Under these conditions, where no cyclic inhibitors are induced, only the false positives will be able to survive.

### Sequential Expression of the Cyclotide-Based Library and the Darwinian-Based Screening System

The different approaches that can be used for the sequential co-expression of library and screening plasmids are summarized in Table 2. The generic vector pASK75 (Skerra (1994) Gene 151(2):131-5) can be used for the inducible expression of the plasmid library. This plasmid carries a tetA promoter region and a tet-repressor encoding gene (tetR) which ensures a tightly regulation in the absence of the corresponding inducer, i.e. tetracycline (Tc) or its anhydro-derivative (aTc). aTc is commercially available and it does not exhibit antibiotic activity, even at concentrations required for full induction. This plasmid also carries a ColE1 origin of replication and a β-lactamase gene.

The split VMA intein-Barnase screening system can be expressed using the DUET expression system. This family of vectors (see Table 2) contains two multi-cloning sites (MCS), each of which is preceded by a T7 promoter/lac operator and a ribosome binding site. The vectors of this family carry all different replicons as well as drug resistance genes (Table 2), which make them ideal candidates for the co-expression of the two split VMA intein fragments that form the core of the Darwinian-based screening system. Controlling basal expression of the screening system in the absence of the inducer will be accomplished by supplementing the bacterial media with glucose (1%). Grossman et al. (1998) Gene 209(1-2):95-103; Nilsson et al. (2005) Protein Pept. Lett. 12(8):795-9.

The level of expression of cylotide-based library can be regulated in a very efficient way by adding different concentrations of the inducer Tc or aTc. Controlling the expression level of the screening plasmid will be accomplished by inducing with different concentrations of isopropyl-β-D-thiogalactopyranoside (IPTG). Further control for the expression of this plasmid can be accomplished by using BL21-derived E. coli tuner strains available from Novagen. This bacterial cell line has a lacZY deletion mutation, which allows uniform entry of IPTG in the cells, so that induction with IPTG occurs in a true-concentration dependant fashion.

### Characterization of the Interaction Between EF-CaM Complex and its Cyclotide-Based Antagonists

Initial characterization of the interaction between the EF-CaM complex and a potential cylotide based-antagonist can be performed using fluorescence anisotropy. For this purpose, CaM is selectively labeled at its C-terminus with fluoresceine using “Expressed Protein Ligation” (EPL). Camarero et al. (1998) J. Pept. Res. 51:303-316; Hofmann et al. (2002) Curr. Opin. Biotechnol. 13(4):297-303; David et al. (2004) Eur. J. Biochem. 271(4):663-77. The complex between EF and fluorescent-labeled CaM is titrated with increasing amounts of the corresponding cyclotide. The integrity of the EF-CaM complex is monitored using fluorescence anisotropy. Specific cyclotides can be either recombinantly expressed or chemically synthesized using Fmoc-based solid-phase peptide synthesis. Camarero et al. (2004) J. Org. Chem. 69(12):4145-51; Camarero et al. (2005) Protein Pept. Lett. 12(8):723-8 Inhibition of the adenylate cyclase activity by any potential cyclotide peptide antagonist is also tested using standard enzymatic assays. Drum et al. (2002) Nature 415(6870):396-342; Shen et al. (2005) EMBO. J. 24(5):929-41.

### Experiment No. 3

### A New Combinatorial Approach for the Generation of Cyclotide Antagonists of the MDM2 Oncoprotein as Anticancer Agents

Since it was first discovered, the MDM2 oncoprotein has been validated as a potential target for cancer drug development. MDM2 amplification and/or overexpression occur in a wide variety of human cancers, including breast cancer, several of which can be treated experimentally with MDM2 antagonists. MDM2 interacts primarily with the p53 tumor suppressor protein in an autoregulatory negative feedback loop to attenuate p53's cell cycle arrest and apoptosis functions Inhibition of the p53-MDM2 interaction has been shown to cause selective cancer cell death, as well as sensitize cancer cells to chemotherapy or radiation effects. Consequently, this interaction has been one of the main focus of anticancer drug discovery targeted to MDM2.

Many pharmaceutically important natural products, including several antibiotics and immuno-suppressants, are based on cyclic peptides. Hence, access to backbone cyclic peptides using recombinant DNA expression techniques offers the intriguing possibility of producing large combinatorial libraries of cyclotides (i.e. estable circular microproteins) using the tools of molecular biology. Such an approach would be analogous to peptide phage-display technology, but would have the advantage of producing the more stable cyclic peptides rather than linear peptides or disulfide-based cyclic peptides. Furthermore theses compounds could be produced and screened for activity inside living cells.

In one aspect, this invention provides a new combinatorial approach for the biosynthesis and screening of cyclic peptides inside living cells. This novel approach is useful to identify molecules able to inhibit the p53-MDM2 interaction. Key to this ‘living combinatorial’ approach is the use of a living cell as a micro-chemical factory for both synthesis and screening of potential inhibitors for a given molecular recognition event. This powerful technique has the advantage that both processes synthesis and screening happen inside the cell thus accelerating the whole process. Note that all the combinatorial approaches developed so far rely on in vitro screening processes that are very time consuming and prone to select binders with poor specificity.

In one aspect, this invention provides a methodology for the biosynthesis of cyclic peptide libraries inside living cells. In a separate aspect, it provides a Darwinian approach for screening inside living cells for inhibitors for the p53-MDM2 interactions. In a yet further aspect, it provides a combined methodology for producing cyclic peptides able to inhibit the interaction between p53 and MDM2.

Circular peptides are biosynthesized inside the cell by using recombinant DNA expression techniques, which allow the creation of vast libraries of these compounds. The circularization of the peptide libraries is accomplished by making use of protein splicing, a naturally occurring process which involves the splicing of polypeptide sequences after the translation step. This process is mediated by an intein domain and it can happen in cis or trans. In vivo screening for inhibitors for the p53-MDM2 interaction will be accomplished by using a Darwinian approach, i.e. only those cells able to express inhibitors for the interaction will survive. This new scheme will be based on conditional protein splicing using the artificially split VMA intein.

Activation of the p53 protein protects the organisms against the propagation of cells that carry damaged DNA with potentially oncogenic mutations. MDM2 is the principal cellular antagonist of p53, acting to limit the p53 grow-suppressive function of unstressed cells. Disruption of the complex p53-MDM2 is the pivotal event for p53 activation, leading to p53 induction and its biological function. Because the p53-MDM2 interaction is structurally and biologically very well understood, the design of molecules able to disrupt or prevent it is currently a hotly pursued therapeutic strategy.

This invention provides a new generic combinatorial approach for the fast and efficient identification of cyclic peptides able to inhibit the p53-MDM2 interaction. The great advantage of this approach is that all the processes (i.e. biosynthesis of the library and screening) happen in the cell and therefore no in vitro screening is required. This considerably speeds up the whole process. Furthermore, because the screening process is taking place in a complex media composed by thousand of proteins (i.e. inside the cytoplasma's cell) it is expected that only members of the library with high specificity for the target will be selected. This will minimize the selection of universal binders, a real problem when in vitro screening methods are employed.

p53—Background and Relevance

Since the discovery of its powerful growth suppressive and pro-apoptotic activity, the tumour suppressor p53 has been in the centre of attention of drug hunters. The idea of unleashing the destructive powers of p53 inside cancer cells has become even more attractive after the realisation that p53 is controlled largely by a single master regulator, MDM2, which binds the tumor suppressor and negatively modulates its activity and stability. Therefore, MDM2 antagonists able to release p53 from the inhibitory grip of MDM2 are expected to stabilize and activate the tumor suppressor, leading to cell cycle arrest or programmed cell death (apoptosis) of cancer cells. Such antagonists could represent a novel modality to treat tumors in which p53 has retained its wild-type structure and function. Targeting the physical interaction between p53 and MDM2 has been regarded as the most direct of all p53-activating strategies.

Human MDM2 is a 491-amino acid (aa)-long phosphoprotein that interacts through its NH₂ terminal domain with an α-helix present in the NH₂ terminal transactivation domain of p53. Kussie et al. (1996) Science 274 (5289):948-53. This entails several negative effects on p53. MDM2 binding to the NH₂ terminal transactivation domain of p53 blocks its transcriptional activity directly. Oliner et al. (1993) Nature 362 (6423):85-60. More importantly, MDM2 functions as the E3 ligase that ubiquitinates p53 for proteasome degradation. Haupt et al. (1997) Nature 387(6630):296-9; Kubbutat et al. (1997) Nature 387(6630) 296-9. The biochemical basis of MDM2-mediated inhibition of p53 function was further elucidated by crystallographic data that showed that the amino terminal domain of MDM2 forms a deep hydrophobic cleft into which the transactivation domain of p53 binds, thereby concealing itself from interaction with the transcriptional machinery. Kussie et al. (1996) Science 274(5289):948-53. The direct interaction between the two proteins has been localized to a relatively small (aa 25-109) hydrophobic pocket domain at the NH₂ terminus of MDM2 and a 15-aa amphipathic peptide at the NH₂ terminus of p53. Kussie et al. (1996) Science 274(5289):948-53; Chen et al. (1993) Mol. Cell. Biol. 13(7):4107-14. The minimal MDM2-binding site on the p53 protein was subsequently mapped within residues 18-26. Chen et al. (1993) Mol. Cell. Biol. 13(7):4107-14; Bottger et al. (1997) J. Mol. Biol. 269(5):744-56.

Site-directed mutagenesis has shown the importance of p53 residues Leu14, Phe19, Leu22, Trp23, and Leu26, of which Phe19, Trp23, and Leu26 are the most critical. Chen et al. (1993) Mol. Cell. Biol. 13(7):4107-14; Bottger et al. (1997) J. Mol. Biol. 269(5):744-56 (See FIG. 1). Accordingly, the MDM2-binding site p53 mutants are resistant to degradation by MDM2. Haupt et al. (1997) Nature 387(6630):299-303; Kubbutat et al (1997) Nature 387(6630):299-303; Kubbutat et al. (1997) Mol. Cell. Biol. 17(1):460-8. Similarly, mutations of MDM2 at residues Gly58, Glu68, Val75, or Cys77 result in lack of p53 binding. Freedman et al. (1997) J. Mol. Med. 3 (4)248-59. The interacting domains show a tight keylock configuration of the p53-MDM2 interface. The hydrophobic side of the amphipathic p53 α-helix, which is formed by amino acids 19-26 (with Phe19, Trp23, and Leu26 making contact), fits deeply into the hydrophobic cleft of MDM2. The realization that p53-MDM2 binding involves the interaction of three critical amino-acid residues from p53 with a well-defined hydrophobic pocket on the surface of MDM2 as well as the fact that the interaction surface between both partners is rather small, have raised the hope that identifying small drug-like pharmacological inhibitors for this interaction might be possible. Moll et al. (2003) Cancer Res. 1(14):1001-8; Klein et al. (2004) Br. J. Cancer (2004) 91(8):1415-9.

As noted above, one objective of this invention is the development of a new combinatorial approach for the biosynthesis and screening of cyclic peptide libraries inside living cells. This novel approach will be initially used for finding cyclic peptides able to antagonize the p53-MDM2 interaction.

Key to this new ‘living combinatorial’ approach is the use of a living cell as a micro-chemical factory for both synthesis and screening of potential inhibitors for a given molecular recognition event. This powerful technique has the advantage that both processes synthesis and screening happen inside the cell thus accelerating the whole process. Note that all the combinatorial approaches developed so far rely on in vitro screening processes that are very time consuming and prone to select binders with poor specificity.

Because the hydrophobic p53-MDM2 interaction is structurally and biologically well understood, the design of small lipophilic molecules able to disrupt or prevent the interaction of p53 and MDM2 in wild-type p53 harboring tumors is currently a hotly pursued therapeutic strategy. Most of the different methods employed so far, however, have only provided inhibitors showing modest potency. Vassilev et al. (2004) Science 303(5659):844-8; Vassilev (2004) Cell Cycle 3(4):419-21. More importantly, most of the methods available so far for this task use in vitro screening approaches, which are typically very slow and prone to select poor-specific binders. This invention provides a totally new and revolutionary approach, a living combinatorial approach. This approach uses living cells for the generation of libraries of small drug-like biomolecules, which are then screened inside the cell for activity. The great advantage of this approach is that all the processes (i.e. biosynthesis of the library and screening) happen in the cell and therefore no in vitro screening is required. This considerably speeds up the whole process. Furthermore, because the screening process is taking place in a complex media composed by thousand of proteins (i.e. inside the cytoplasma's cell) it is expected that only members of the library with high specificity for the target will be selected. This will minimize the selection of universal binders, a real problem when in vitro screening methods are employed. We plan to use this approach for finding cyclic peptides able to antagonize the p53-MDM2 interaction.

This invention uses a library of living cells. Each individual cell within this living library will be able to biosynthesize a unique small drug-like biomolecule. This compound is then screened for activity inside the cell. To this end, this invention also provides, 1) a method for the biosynthesis of libraries of cyclic peptides and 2) and efficient method for carrying out the screening process inside the cell and be able to report the results.

### Biosynthesis of Circular Polypeptides

The compounds used in the libraries for in vivo screening are cyclic polypeptides. Cyclic peptides are one the most common scaffolds used in nature to produce high affinity drug-like effectors (eg. antibiotics, immuno-supressants, etc). Indeed, peptide cyclization is commonly used in medicinal chemistry for modifying the properties of bioactive peptides. Kessler (1982) Angew. Chem. Int. Ed. Engl. 21:512-523; Hruby et al. (1990) J. Biochem 268:249-262. In particular, backbone (head-to-tail) cyclization has been widely used to rigidify peptide structure, therefore minimizing the entropic cost of receptor binding, and to improve the in vivo stability of peptides.

Cyclic peptides will be biosynthesized inside the cell by using recombinant DNA expression techniques. This allows the creation of vast libraries of these compounds. The cyclization of the peptide libraries is accomplished by making use of protein splicing (Camarero et al. (1999) J. Am. Chem. Soc. 121:5597-5598; Scott (1999) Proc. Natl. Acad. Sci. USA 96(24):13638-13643; Camarero et al. (2001) Bioorg. Med. Chem. 9(9):249-84), a naturally occurring process which involves the splicing of polypeptide sequences after the translation step (see FIG. 15). This process is mediated by an intein domain (grey sequence in FIG. 15) and it can happen in cis or trans.

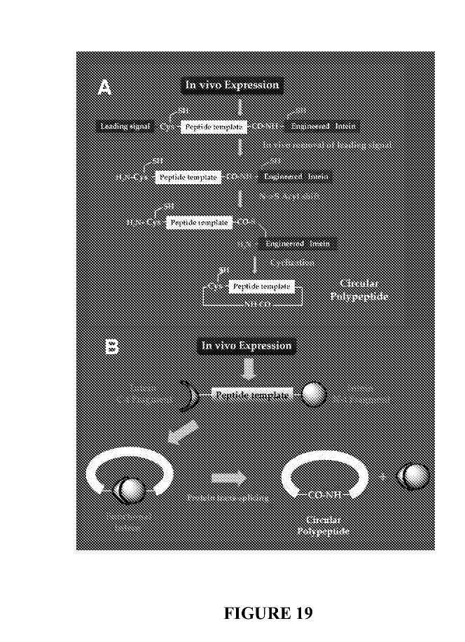

This invention contemplates two complementary approaches for the biosynthesis of libraries of circular peptides in vivo based on this natural process (see FIG. 19). The first approach uses an engineered intein unit and a leading polypeptide sequence to introduce a C-terminal α-thioester and a N-terminal Cys residue at the peptide sequence to be cyclized. These two chemical moieties will react very efficiently with each other to yield the corresponding circular peptide. Camarero et al. (1999) J. Am. Chem. Soc. 121:5597-5598; Camarero et al. (2001) Bioorg. Med. Chem. 9(9):2479-84; Camarero (1997) J. Chem. Soc., Chem. Comm. 1369-1370; Camarero (1998) Angew. Chem. Int. Ed. 37(3):347-349 (FIG. 19A). The second approach is based on the use of protein trans-splicing. Scott et al. (1999) Proc. Natl. Acad. Sci. USA 96(24):13638-13643; Scott (2001) Chem. Biol. 8(8):801-815; Abel-Santos (2003) Methods Mol. Biol. 205:281-294 (FIG. 19B). In this case the N-terminal and C-terminal intein fragments are fused to the C- and N-terminus of the peptide to be cyclized, respectively. Thus, when the corresponding chimeric precursor is expressed, the two intein fragments will associate spontaneously allowing protein splicing to occur. This event will produce the circularization of the corresponding peptide sequence.

### Library Design

This invention creates our library of circular peptides using a cyclotide scaffold. Cyclotides are a newly emerging family of large backbone cyclic polypeptides (≈30 residues long) characterized by a disulfide-stabilized core (3 disulfide bonds) with an unusual knotted structure (FIG. 4). Craik et al. (1999) J. Mol. Biol. 294(5):281-294; Trabi et al. (2002) Trends Biochem. Sci. 2002 27(3):132-8; Craik et al. (2004) Curr. Protein Pept. Sci. 5(5):297-315; Goransson et al. (2004) Curr. Protein Pept. Sci. 5(5):317-29; Vogel et al. (2005) Structure (Camb) 13(5):688-90. In contrast to other cyclic polypeptides, cyclotides have a well-defined three-dimensional structure. Therefore, despite their small size, they can be considered mini-proteins. The unique cyclic-backbone topology and knotted arrangement of 3 disulfide bonds endow cyclotides with exceptional stablity and resistance to chemical, enzymatic and thermal degradation. Goransson et al. (2003) J. Biol. Chem. 278(48):48188-96; Colgrave et al. (2004) Biochemistry 43(20):5965-75. Furthermore, their well-defined structures have been associated with a range of biological functions such as uterotonic activity, inhibition of trypsin and neurotension binding, cytotoxicity, anti-HIV, antimicrobial, and insecticidal activity. Gran (1973) Acta. Pharmacol. Toxicol. (Copenh) 33(5):400-8; Gran (1973) Lloydia 36(2):174-8; Witherup et al. (1994) J. Nat. Prod. 57(12):1619-25; Gustafson et al. (2004) Curr. Protein Pept. Sci. 5(5):331-40; Tam et al (1999) Proc. Natl. Acad. Sci. USA 96(16):8913-8; Jennings et al. (2001) Proc. Natl. Acad. Sci. USA 98(19):10614-9; Felizmenio-Quimio et al. (2001) J. Biol. Chem. 276(25):22875-82. Together, these characteristics suggest that cyclotides are ideal molecular scaffolds for the development of stable peptide drugs. Craik et al. (2004) Curr. Protein Pept. Sci. 5(5):297-315; Craik (2002) Curr. Opin. Drug Discov. Devel. 5(2):251-260. This library will be created at the DNA level using degenerated DNA oligos coding for some of the different loops of the cyclotide scaffold (i.e. loops 2, 3 and 5, see FIG. 10). In order to facilitate the analysis of the in vivo cyclization products, a small CBD affinity tag will be added to the C-terminus of the corresponding linear precursor fusion proteins.

### In Vivo Screening

The screening process for finding which cyclic peptide within the library are able to inhibit p53-MDM2 interaction will be carried out in vivo by using a Darwinian selection. Key to this approach is the use of conditional protein splicing (CPS). Mootz et al. (2003) J. Am. Chem. Soc. 125(35):10561-9; Mootz (2002) J. Am. Chem. Soc. 124(31)a:9044-5. In this approach, a naturally occurring intein is artificially split into two fragments (N-intein and C-intein). In contrast with naturally split inteins, these two fragments do not have affinity for each other and in absence of any other interaction they remain inactive, i.e. unable to produce protein splicing. It has been shown, however, that when two interacting protein domains are fused to the C- and N-termini of the N- and C-intein moieties, respectively, the binding event is able to bring together both intein fragments allowing them to fold and become active. This event will trigger protein splicing and concomitant ligation of both N- and C-extein polypeptides. Applicants' in vivo screening approach (see Scheme 2) uses the artificially split VMA intein system (VMA-N-intein (aa 1-184) and VMA-C-intein (aa 390-454). Mootz et al. (2003) J. Am. Chem. Soc. 125(35):10561-9; Mootz (2002) J. Am. Chem. Soc. 124(31)a:9044-5. The Barnase N-terminal (aa 1-66) and C-terminal fragments (aa 67-110) are used as N- and C-extein polypeptides, respectively (Barnase is a powerful toxin when is expressed and properly folded inside cells, see FIG. 5). This creates an expression system that will only produce the full length cytotoxic Barnase if the split intein becomes active. In order to screen in vivo potential antagonists for the interaction p53-MDM2, the transactivation domain of human p53 (aa 15-30) will be fused to the N-terminus of the VMA-C-intein-Barnase (aa 68-110) fusion protein and the N-terminal MDM2 domain (aa 1-108) will be also fused to the C-terminus of the Barnase (aa 1-67)-VMA-N-intein fusion protein. The expression of these two fusion proteins inside cells will provide a novel in vivo Darwinian screening approach for finding antagonists to the p53-MDM2 interaction. Only those cells being able to biosynthesize high affinity antagonists will be able to prevent the formation of Barnase and therefore survive.

Combining Biosynthesis of Cyclic Peptide Libraries with In Vivo Darwinian Screening

This invention combines both approaches, i.e. biosynthesis of libraries with in vivo screening methods using E. coli cells. This involves the creation of two plasmids, the library and the screening plasmid. The library plasmid will be in fact a library of plasmids (in excess of 10⁹ members). All the plasmids of this library will contain a variable DNA sequence codifying for the different cyclic peptides that will present on the library. This variable sequence will be fused to the appropriate engineered intein element in order to allow its cyclization once expressed inside the cell. The screening plasmid will contain the two VMA split intein fusion proteins containing the Barnase fragments as well as the p53-MDM2 interaction polypeptide domains. Both plasmids will contain different promoter regions (for example arabinose, tetracycline and/or T7 promoters) in order to control the level of expression of the cyclic peptides and the split VMA intein fusion proteins involved in screening. Therefore only those cells being able to survive, when both plasmids are expressed, will contain potential positives. Discrimination between false and real positives is accomplished by activating only the expression of the screening plasmid. Under these conditions, where no cyclic inhibitors are induced, only the false positives will be able to survive.

### Characterization of the Interaction Between MDM2 and its Cyclotide-Based Antagonists

Characterization of the inhibition interaction between MDM2 and a potential cylotide based-antagonist is performed using fluorescence anisotropy. For this purpose the MDM2 protein domain will be selectively labeled at its C-terminus with fluoresceine using “Expressed Protein Ligation” (EPL). Camarero et al. (1998) J. Pept. Res. 51:303-316; Hofmann et al. (2002) Curr. Opin. Biotechnol. 13(4):297-303; David et al. (2004) Eur. J. Biochem. 271(4):663-77. The structural characterization of the interaction can, in one aspect, be completed using heteronuclear NMR spectroscopy. Mapping the potential interaction between MDM2 and its inhibitor can be carried using the chemical shift perturbation/SAR-by-NMR technique. Shuker et al. (1996) Science 274(5292):1531-4; Camarero et al. (2002) Proc. Natl. Acad. Sci. USA 99(13):8536-8541. This is accomplished by titrating a ¹⁵N-labeled MDM2 sample with the corresponding cyclotide-based antagonist. Since the ¹H and ¹⁵N chemical shifts for the human MDM2 p53 binding domain are known (Uhrinova et al. (2005) J. Mol. Biol. 350(3):587-98), it will be possible to determine the binding site for the antagonist.

The ability to create cyclic polypeptides in vivo opens up the possibility of generating large libraries of cyclic polypeptides. Using the tools of molecular biology, genetically encoded libraries of cyclic polypeptides containing billions of members can be readily generated. This tremendous molecular diversity forms the basis for selection strategies that model natural evolutionary processes. Also, since the cyclic polypeptides are generated inside living cells, these libraries can be directly screened for their ability to attenuate or inhibit cellular processes.

In contrast to phage display, where the screening takes place in vitro, screening that takes place in the cytoplasm offers the advantages conferred by a native physiological environment where diverse biochemical events may be examined. In addition, problems resulting from the presence of a fusion tag (in this case the viral particle), in a phenomenon known as template effect, may be circumvented.

Cyclic polypeptides are relatively more stable and more resistant to cellular catabolism than linear polypeptides or disulfide-based cyclic polypeptides. Naturally occurring cyclic peptides often exhibit diverse therapeutic activities ranging from immuno-suppression to antimicrobial activity. The stability of backbone cyclized polypeptides that display certain pharmacological properties suggests that they may be suitable scaffolds on which to graft the molecular diversity of an intracellular library. In particular, the cyclotide scaffold is used for the in vivo generation of molecular diversity. As stated before, cyclotides are an extremely interesting novel class of circular, disulfide-rich peptides that display a broad range of bioactivities and have exceptionally high stability to extreme conditions. Their physical properties, which include resistance to thermal and enzymatic degradation, can be attributed to their unique cyclic backbone and knotted arrangement of disulfide bonds. These exceptional characteristics make them ideal templates for drug design and discovery. Access to biosynthetic cyclotides using recombinant DNA expression techniques offers the exciting possibility of producing large combinatorial libraries of highly stable miniproteins. This allows the generation of cell-based combinatorial libraries that could be screened either in vitro or in vivo for their ability to regulate cellular processes.

This invention provides the combined use of biosynthetically generated cyclotide-based libraries and in vivo screening methods to find potential antagonists for the p53-MDM2 interaction. The MDM2 protein, which was originally identified as an oncogene, has been shown to be upregulated in many human breast tumors and carcinomas, soft tissue sarcomas, and other cancers. Iwakuma et al. (2003) Mol. Cancer. Res. 1(14):993-1000. The main cellular function of MDM2 is to regulate p53 levels. Principally, MDM2 promotes p53 degradation through an ubiquitin-dependent pathway. The tumor suppressor p53 is a potent transcription factor, which is activated following cellular stress and regulates multiple downstream genes involved in cell cycle and apoptosis hence playing a pivotal role in protection from cancer. The interaction between p53-MDM2 has been also biologically and structurally very well studied. All of this has made the p53-MDM2 interaction a very promising therapeutical target for cancer treatment and prevention. Klein et al. (2004) Br. J. Cancer 91(8):1415-9; Vassilev (2004) Cell Cycle 3(4):419-21.

The compositions and methods of this invention can generate cyclotide-based antagonists to provide potential peptide-based drugs useful for the treatment of some types of cancer. Due the relatively small size of cyclotide peptides, these compounds can be easily chemically synthesized (Daly et al. (1999) Biochemistry (1999) 38(32):10606-14) using intramolecular “native Chemical Ligation” (Camarer et al. (1999) J. Am. Chem. Soc. 121:5597-5598; Camarero et al. (1997) J. Chem. Soc., Chem. Comm. 1997:1369-1370; Camarero et al. (1998) Angew. Chem. Int. Ed. 37(3):347-349) and consequently chemically modified to contain membrane translocation domains (PTD). This will facilitate their translocation inside the cell and allow its inhibitory role in vivo. One of the most commonly used PTDs is the peptide penetratin, a small 16-residue polypeptide derived from the third helix of the homeodomain of the Antennapedia protein. Derossi et al. (1996) J. Biol. Chem. 271(30):18188-93; Christiaens et al. (2004) Eur. J. Biochem. 271(6):1187-97; Dietz et al. (2004) Mol. Cell. Neurosci. 27(2):85-131. This small peptide is able to translocate polypeptides and proteins across biological membranes (including the nuclear membrane) by an energy-independent mechanism. More importantly, D-based penetratin peptides have been also shown to be active in their ability to translocate polypeptides. Derossi et al. (1996) J. Biol. Chem. 271(30):18188-93. Hence, it is anticipated that high affinity cyclotide-based MDM2 antagonists chemically conjugated to D-based PTDs could be used as potential drugs against certain types of cancer. The high stability of the cyclotide scaffold linked to the D-nature of the PTD will ensure a high bioavailability for these compounds.

### Experiment No. 4

### Biosynthesis and Functional Screening of a MCoTI-I Based Genetically Encoded Library

This example reports the biosynthesis and screening of biological activity of libraries based on the cyclotide MCoTI-I. These libraries were designed to contain multiple MCoTI-I mutants, in which all the residues in loops 1, 2, 3, 4 and 5, except for the Cys residues involved in the cystine-knot, were replaced by different types of amino acid. These mutations included the introduction of neutral (Ala), flexible and small (Gly), hydrophilic (Ser and Thr), hydrophobic (Met and Val), constrained (Pro) and aromatic (Tyr and Trp) residues (see Table 3). The only residue in loop 6 that was mutated was Val¹. This residue is a hydrophobic b-branched amino acid highly conserved in other squash trypsin inhibitors (STIs) and is in close proximity to Lys⁴ in loop 1, which is responsible for MCoTI ability to inhibit trypsin. The rest of the residues in loop 6 are not required for folding or biological activity in linear STIs (Avrutina et al. (2005) Biol. Chem. 386(12):1301-1306) and therefore were not explored. It is believed that loop 6 acts as a very flexible linker to allow cyclization. Heitz et al. (2001) Biochemistry. 40(27):7973-7983. To Applicant's knowledge this is the first time that a cyclotide-based library is biosynthesized in E. coli cells and a complete amino acid scanning is carried out in the cyclotide MCoTI-I to explore the effects of individual amino acids on biological activity and structural requirements.

The biosynthesis of MCoTI-I mutants was carried out by using a protein splicing unit in combination with an in-cell intramolecular native chemical ligation reaction (NCL) (FIG. 3). Kimura et al. (2006) Angewandte Chemie (International ed.) 45(6):973-976; Camarero et al. (2007) Chembiochem. 8(12):1363-1366. Intramolecular NCL requires the presence of an N-terminal Cys residue and a C-terminal α-thioester group in the same linear precursor molecule. Camarero et al. (1997) J. Chem. Soc., Chem. Comm. 1997:1369-1370; Camarero & Muir (1999) J. Am. Chem. Soc. 121:5597-5598. For this purpose the MCoTI-I linear precursors were fused in frame at their C- and N-terminus to a modified Mxe Gyrase A intein and a Met residue, respectively. This allows the generation of the required C-terminal thioester and N-terminal Cys residue after in vivo processing by endogenous Met aminopeptidase (MAP). The native Cys located to the beginning of loop 6 to facilitate the cyclization was used. This linear construct has shown to give very good expression and cyclization yields in vivo. Camarero et al. (2007) Chembiochem. 8(12):1363-1366.

In order to facilitate the analysis and processing of all the mutants, two libraries (Lib1 and Lib2) were produced containing 13 and 15 different MCoTI-I mutants (see Table 3). These libraries were designed to contain mutants that could be easily identified by ES-MS. In both libraries the MCoTI-I wild-type (wt) sequence was included as control. Synthetic dsDNA fragments encoding the different MCoTI-I mutants were ligated into plasmid pTXB1 in frame with Mxe Gyrase intein (Table 4). The resulting plasmid libraries were transformed into competent DH5α E. coli cells obtaining approximately 10⁴ colonies (data not shown). All colonies were pooled and the corresponding plasmid library was transformed into E. coli Origami2(DE3) for protein overexpression.

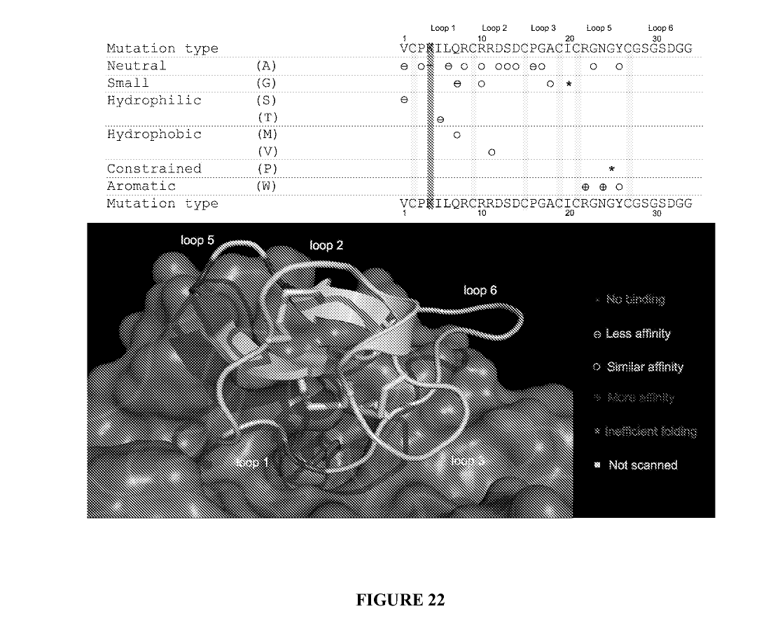

Expression of the library in E. coli produced the corresponding MCoTI mutants-Gyrase intein linear fusion precursors with similar yields to that of the wild-type MCoTI-I wt. Camarero et al. (2007) Chembiochem. 8(12):1363-1366. The level of in vivo cleavage was estimated to be ≈80% following induction for 20 h at 20° C. (FIG. 22). These expression conditions maximize the in vivo processing of the linear intein-fusion precursors to give natively folded MCoTI cyclotides. Camarero et al. (2007) Chembiochem. 8(12):1363-1366. In vivo cleavage and processing of the corresponding intein linear fusion precursor can be reduced by inducing at relatively elevated temperatures for short times (30° C. for 2-4 h for example) while keeping a similar level of protein expression (FIG. 22). This allows Applicant to vary the amounts of folded MCoTI mutants produced to access different screening methods. For in vitro screening, cyclization can be accomplished in vitro under controlled conditions, and therefore short induction times at relatively higher temperatures will yield more uncleaved linear precursor. Alternatively, in vivo cyclization yields can be easily maximized by using longer induction times and lower induction temperatures (20° C. for 20 h, for example) for high throughput in vivo screening,

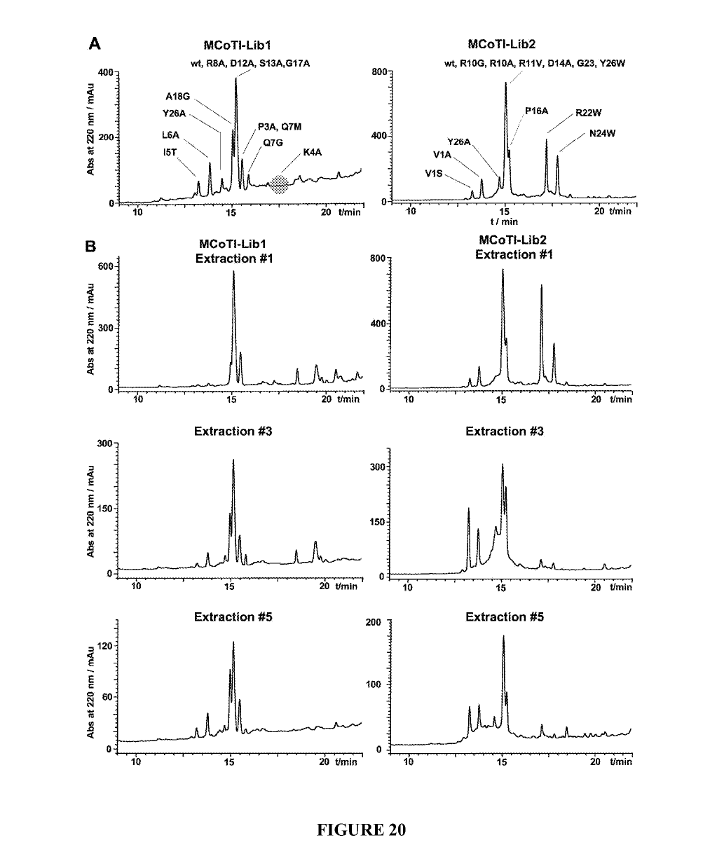

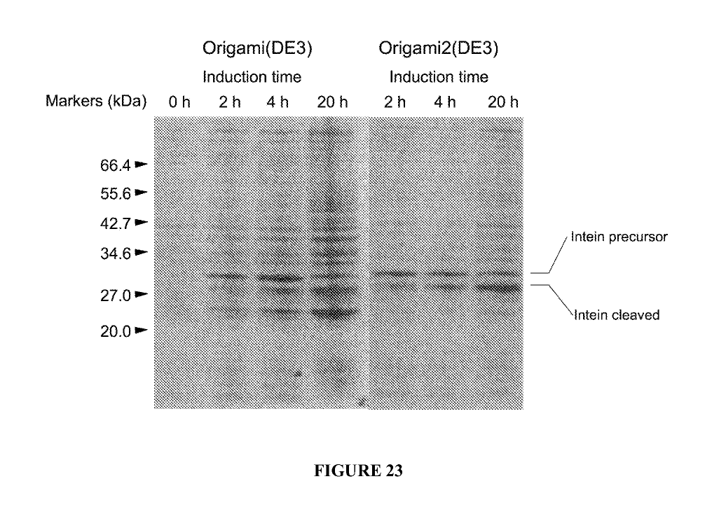

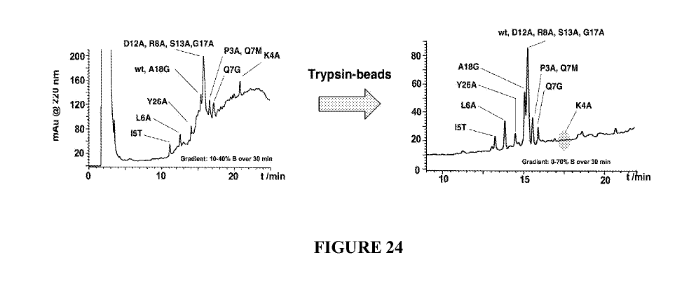

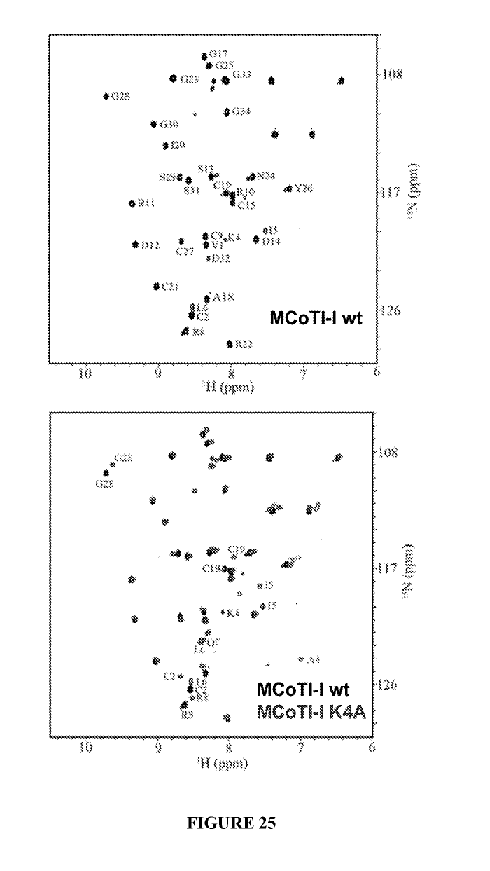

In order to characterize the MCoTI-based libraries and assess structural integrity of MCoTI mutants, Applicants used the biological activity of MCoTI to bind trypsin. Purified MCoTI mutants-Gyrase fusion proteins were obtained from E. coli Origami2(DE3) cells which were induced at 30° C. for 4 h. Under these conditions only about 30% of the intein linear precursors were processed in vivo. The fusion precursors were cleaved and cyclized in phosphate buffer at pH 7.2 containing 50 mM GSH for 36 h. In Applicant's hands, GSH has been shown to be more effective than other thiols to promote cyclization and correct folding of cyclotides and other disulfide containing peptides in vitro. Kimura et al. (2006) Angewandte Chemie (International ed.) 45(6):973-976; Camarero et al. (2007) Chembiochem. 8(12):1363-1366; Austin et al. (2010) Amino Acids. 38(5):1313-1322. This treatment resulted in nearly 100% cleavage of the intein precursors. The soluble fractions were purified on trypsin-sepharose beads and the bound fractions analyzed by HPLC and ES-MS to determine the presence of relative representation of the library members able to bind trypsin (FIG. 20A). As anticipated, the MCoTI-K4A mutant was not found in the trypsin-bound fraction. This residue determines binding affinity and specificity, and can only be replaced by Arg to maintain biological activity. Hernandez et al. (2000) Biochemistry. 39(19):5722-5730. Analysis of the cyclization reaction before affinity purification confirmed the presence of this mutant in the corresponding library (FIG. 23). The K4A mutant was also individually cyclized, purified and characterized by NMR showing a native cyclotide topology when to compared to MCoTI-I wild type (FIG. 24 and Table 5) therefore indicating that the lack of biological activity of this mutant was due to the replacement of Lys⁴ by Ala, and not to the adoption of a non-native fold. The mutant MCoTI-I G25P was also absent in the trypsin bound fraction. In vitro cyclization of the G25P revealed that the intein precursor of this cyclotide was not processed efficiently and the resulting cyclotide was not able to fold properly. Only traces of natively folded G25P were detected in the GSH-induced cyclization/folding of the corresponding intein precursor (FIG. 25). The inefficient cleavage of this mutant precursor could be explained by the proximity of a Pro residue to the MCoTI-intein junction, which may affect the ability of the Gyrase intein to produce the thioester intermediate required for the intramolecular cyclization. The Gly²⁵ residue is located at loop 5 and it is extremely well conserved in all the STIs thus corroborating the importance of this residue for correct folding of MCoTI cyclotides.

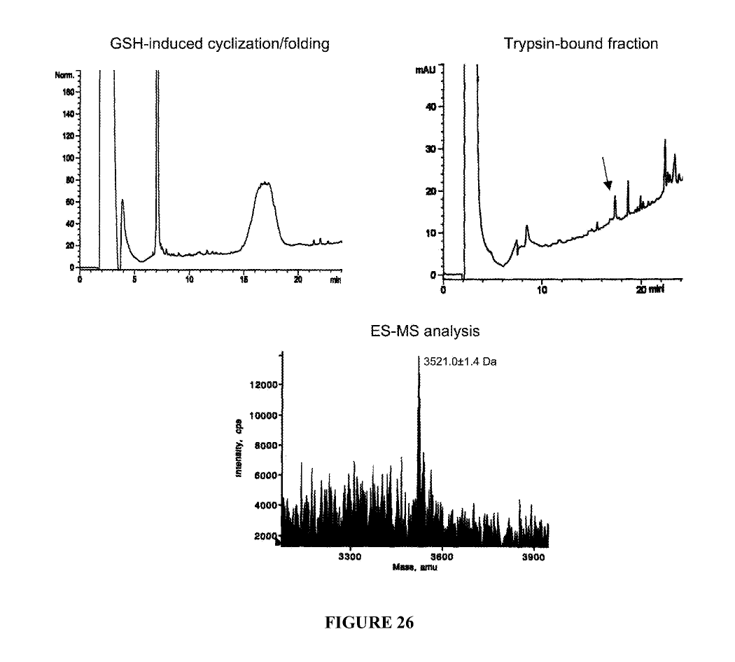

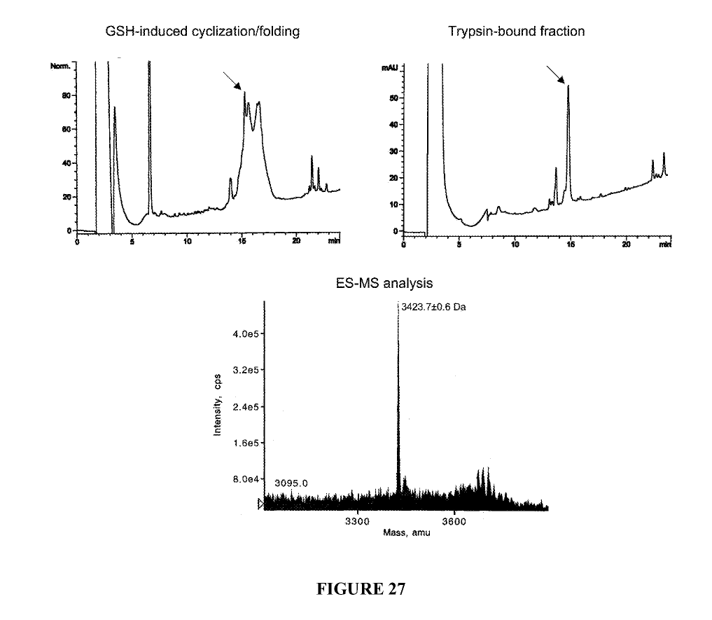

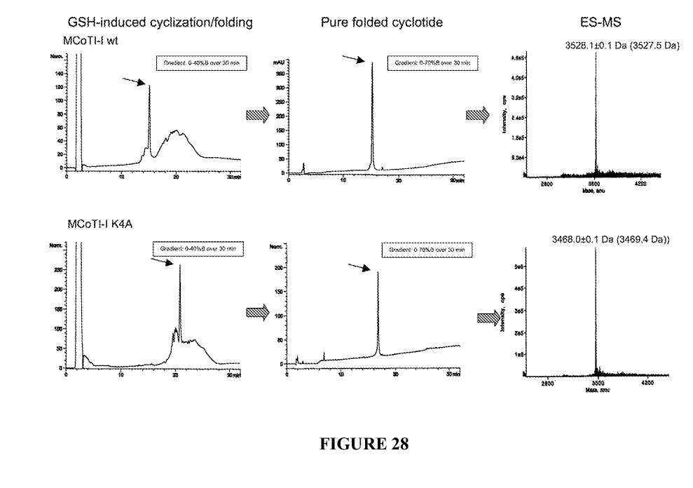

Remarkably, the remaining mutants were identified on the trypsin bound fraction indicating that the corresponding mutants were able to adopt a native-like structure and that its ability to bind trypsin was not significantly disrupted. All the active mutants besides I20G were produced with similar yields to the MCoTI-wt (within 50% of the average value), as quantified by HPLC and ES-MS. The folded 120G mutant abundance was estimated to be about 10% of the average. Cleavage and cyclization of 120G using GSH revealed that although the thiol-induced cleavage was very efficient, the correctly folded mutant was produced in very low yield (FIG. 26) indicating the importance of this residue for efficient folding in MCoTI cyclotides. In fact this residue, which is located between the Cys residues at the end and beginning of loops 3 and 5, respectively, is well conserved among the different linear STIs and cyclotides showing a preference for b-branched residues and hydrophilic residues. Interestingly, folded I20G mutant was able to bind trypsin beads confirming the ability to adopt a native folded structure.

Next, the biological activity of the MCoTI-I libraries produced in vivo were screened. For this purpose both libraries (Lib1 and Lib2) were expressed in E. coli Origami2(DE3) cells at 20° C. for 20 h in order to maximize intracellular processing and folding of the different intein precursors. After lysing the cells by sonication, the cellular supernatant was purified using trypsin-sepharose under competing binding conditions as described above. The different fractions were then analyzed and quantified by HPLC and ES-MS. The results obtained were very similar to those found with in vitro cyclized libraries (data not shown), thus indicating that the composition of the libraries obtained in vitro and in vivo were practically identical.

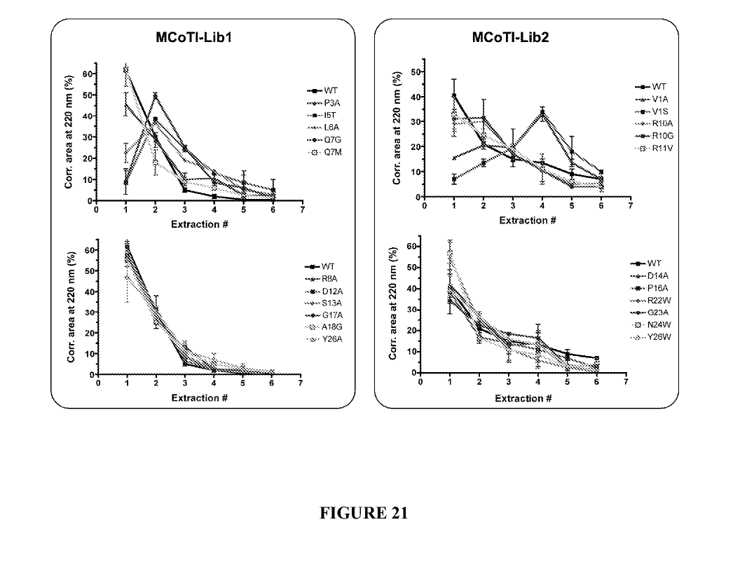

In order to establish the relative affinities of the different mutants able to bind trypsin versus MCoTI-I wild type, in vitro and in vivo cyclized libraries were incubated with trypsin-sepharose under competing conditions, i.e. using only about 20% of the required trypsin-sepharose beads for stoichiometric binding. This process ensured that cyclotides with tighter affinities competed for binding to trypsin leaving the members of the library with weaker affinities in the superantant (i.e. unbound fraction). This supernatant was then purified again using the same approach to extract the remaining active cyclotides. This process was repeated several times until all the active cyclotides found in a particular library sample were completed extracted. This process ensured that cyclotides with tighter affinities for trypsin were extracted during the first affinity purifications leaving the library members with weaker affinities to be purified later on this sequential extraction process. All the different trypsin-bound fractions were then analyzed and quantified using HPLC and ES-MS (FIG. 20B). The results, summarized in FIG. 21, show that using MCoTI-I wt as internal reference, the mutants N24W and R22W were consistently able to compete slightly with the rest of mutants (including wt) thus indicating a somewhat tighter affinity for trypsin than the wt sequence. Most of the remaining mutants: P3A, Q7M, RBA, R10A, R10G, R11V, D12A, S13A, D14A, P16A, G17A, A18G, G23A, Y26A and Y26W showed similar elution profiles to that of wt indicating a similar affinity for trypsin. Mutants VIA, V1S, I5T, L6A and Q7G, on the other hand, were consistently extracted after MCoTI wt indicating a lower affinity for trypsin than the wt sequence. I20G was also extracted after MCoTI-I wt, however this could be due to the low abundance of folded cyclotide.

Although there is not a structure available for the complex between MCoTI cyclotides and trypsin, the structure of several complexes formed between different STIs and trypsin have been reported so far. Based on the high sequence homology between these trypsin inhibitors and MCoTI cyclotides (FIG. 2A), it is reasonable to assume that they possess the same binding mode to trypsin. Hernandez et al. (2000) Biochemistry. 39(19):5722-5730. Therefore, it is not surprising that mutant K4A was not able to bind trypsin since K4 is critical for binding to the trypsin specificity pocket. Hernandez et al. (2000) Biochemistry. 39(19):5722-5730. Other mutations in loop 1 also affected negatively trypsin binding. Hence, mutants I5T, L6A and Q7G were consistently eluted in the later fractions in our competing binding experiments, indicating a weaker affinity for trypsin. The sequence Lys/Arg-Ile-Leu in loop 1 is extremely well conserved in all linear STIs suggesting that it is required for efficient trypsin binding. Position 7, on the other hand, seems to be more promiscuous and it is able to accept hydrophobic residues (Q7M showed a similar elution pattern than MCoTI-wt) and positively charged residues (cyclotide MCoTI-II has a Lys residue in this position), but not a small and flexible residue like Gly (mutant Q7G shows weaker trypsin affinity than wt). Also in loop 1, mutation R8A did not affect significantly trypsin-binding and the corresponding mutant showed an elution pattern similar to that of wt. In agreement with this result, this position is not especially well conserved in linear STIs allowing the presence of charged (both positive and negatively charged) and Pro residues. All the mutations explored in loop 2 had similar elution patterns to the wt sequence. This should be expected since this loop is solvent exposed and on the opposite side to loop 1.

The only mutation affecting trypsin binding in loop 3 was represented by mutant P16A. The rest of the mutants in this loop behaved similar to the wt sequence. This loop is partially exposed in the structure of several linear STIs with trypsin and it shows significant sequence heterogeneity among the different STIs. Position 16, however, is usually occupied in other STIs by hydrophobic residues (mainly Leu and Met), which could explain the observed behavior of mutant P16A. None of the mutations in loop 5, besides G25P, had an adverse effect on trypsin binding. It is interesting to remark that mutants Y26A and Y26W showed a similar elution profile to the wt sequence (FIG. 21). This position is very well conserved among different STIs being occupied mainly by either aromatic (Tyr or Phe) or in some cases Ile and His. Analysis of the structure of linear Cucurbita pepo typsin inhibitor-II (CPTI-II, which shares ≈75% sequence homology with MCoTI-I) complexed with bovine trypsin shows that this position makes a direct contact with trypsin Tyr¹⁵¹ residue, which is highly conserved among different trypsin homologs. Intriguingly, mutants R22W and N24W seemed to slightly outcompete the rest of the library members including MCoTI-I wt in our binding competing experiments (FIGS. 20B and 21). These residues are in close proximity to Tyr²⁶ and they could help to further stabilize the aromatic interaction described before between the MCoTI-I mutants and trypsin.

The position corresponding to Val^{I} at the end of loop 6 was also explored by including mutants V1A and V1S in this study. This position favors the presence of hydrophobic residues (mainly Val, Met and Ile) among different STIs, although hydrophilic and charged residues are also found in some STIs. Visual inspection of the CPTI-II-trypsin complex, for example, reveals that this position is in close proximity to trypsin. Trp²¹⁵. This aromatic residue is highly conserved among the different trypsin homologs indicating that this interaction may be important to the stabilization of the complex. Consistent with this finding, replacement of Val¹ in MCoTI-I by Ser and Ala produced mutants that consistently showed a weaker affinity than MCoTI-I wt.

In summary, these data provide significant insights into the structural constraints of the MCoTI cyclotide framework and the functional elements for trypsin binding. To the best of Applicant's knowledge, this is the first time that the biosynthesis of a genetically-encoded library of MCoTI-based cyclotides containing a comprehensive suite of amino acid mutants is reported. Others working in this technology have also recently reported the chemical synthesis of a complete suite of Ala mutants for the cyclotide Kalata B1 (KB-1). These mutants were fully characterized structurally and functionally. Their results indicated that only two of the mutations explored (KB-1 W20A and P21A, both located in loop 5, sec FIG. 2) prevented folding. The mutagenesis results obtained in Applicant's work show similar results highlighting the extreme robustness of the cyclotide scaffold to mutations. Only two of the 27 mutations studied in the cyclotide MCoTI-I, G25P and I20G, affected negatively the adoption a native cyclotide fold. Intriguingly, the rest of the mutations allowed the adoption of a native fold as indicated by ES-MS analysis and their ability to bind trypsin (or NMR in the case of K4A). These results should provide an excellent starting point for the effective design of MCoTI-based cyclotide libraries for rapid screening and selection of de novo cyclotide sequences with specific biological activities.

The libraries used in this work were produced either in vitro by GSH-induced cyclization/folding or by in vivo self-processing of the corresponding precursor proteins. In both cases the results were similar indicating that this approach is quite general for the production of complex libraries. Importantly, in vivo biosynthesis of cyclotide-based libraries may have tremendous potential for drug discovery. This study shows that MCoTI-cyclotides may provide an ideal scaffold for the biosynthesis of large combinatorial libraries inside of living E. coli cells. Coupled to an appropriate in vivo reporter system, this library may rapidly be screened using high throughput technologies such as fluorescence activated cell sorting. Kimura et al. (2007) Anal. Biochem. 369(1):60-70; Sancheti & Camarero (2009) Adv. Drug Deliv. Rev. 61(11):908-917.

TABLE 3
Sequences and molecular weights found for the different
MCoTI-I mutants used in this work.

| | | Molecular weight (Da) | |
|---|---|---|---|
| Name | Sequence | Expected | Found |
| Lib1 | | | |
| wt | CGSGSDGGVCPKILQRCRRDSDCPGACICRGNGY | 3480.94 | 3480.4 ± 0.6 |
| P3A | CGSGSDGGVCAKILQRCRRDSDCPGACICRGNGY | 3454.91 | 3454.2 ± 0.3 |
| K4A | CGSGSDGGVCPAILQRCRRDSDCPGACICRGNGY | 3423.85 | 3423.7 ± 0.6 |
| I5T | CGSGSDGGVCPKTLQRCRRDSDCPGACICRGNGY | 3468.89 | 3468.0 ± 0.1 |
| L6A | CGSGSDGGVCPKIAQRCRRDSDCPGACICRGNGY | 3438.86 | 3438.7 ± 1.0 |
| Q7G | CGSGSDGGVCPKILGRCRRDSDCPGACICRGNGY | 3409.87 | 3408.5 ± 1.2 |
| Q7M | CGSGSDGGVCPKILMRCRRDSDCPGACICRGNGY | 3484.01 | 3483.7 ± 0.6 |
| R8A | CGSGSDGGVCPKILQACRRDSDCPGACICRGNGY | 3395.84 | 3396.0 ± 0.1 |

| | | | |
|---|---|---|---|
| D12A | CGSGSDGGVCPKILQRCRRASDCPGACICRGNGY | 3436.93 | 3435.7 ± 0.6 |
| S13A | CGSGSDGGVCPKILQRCRRDADCPGACICRGNGY | 3464.95 | 3465.0 ± 1.0 |
| G17A | CGSGSDGGVCPKILQRCRRDSDCPAACICRGNGY | 3494.97 | 3495.0 ± 1.0 |
| A18G | CGSGSDGGVCPKILQRCRRDSDCPGGCICRGNGY | 3466.92 | 3467.0 ± 1.7 |
| Y26A | CGSGSDGGVCPKILQRCRRDSDCPGACICRGNGA | 3388.85 | 3388.3 ± 1.5 |

| Lib2 | | | |
|---|---|---|---|
| wt | CGSGSDGGVCPKILQRCRRDSDCPGACICRGNGY | 3480.94 | 3480.4 ± 0.6 |
| V1A | CGSGSDGGACPKILQRCRRDSDCPGACICRGNGY | 3452.89 | 3453.0 ± 1.0 |
| V1S | CGSGSDGGSCPKILQRCRRDSDCPGACICRGNGY | 3468.89 | 3468.7 ± 1.2 |
| R10A | CGSGSDGGVCPKILQRCARDSDCPGACICRGNGY | 3395.84 | 3396.0 ± 0.1 |
| R10G | CGSGSDGGVCPKILQRCGRDSDCPGACICRGNGY | 3381.81 | 3380.3 ± 0.6 |
| R11V | CGSGSDGGVCPKILQRCRVDSDCPGACICRGNGY | 3423.89 | 3423.7 ± 0.6 |
| D14A | CGSGSDGGVCPKILQRCRRDSACPGACICRGNGY | 3436.93 | 3436.7 ± 1.2 |
| P16A | CGSGSDGGVCPKILQRCRRDSDCAGACICRGNGY | 3454.91 | 3455.0 ± 1.7 |
| I20G | CGSGSDGGVCPKILQRCRRDSDCPGACGCRGNGY | 3424.84 | 3423.7 ± 0.6 |
| R22W | CGSGSDGGVCPKILQRCRRDSDCPGACICWGNGY | 3510.97 | 3510.7 ± 1.2 |
| G23A | CGSGSDGGVCPKILQRCRRDSDCPGACICRANGY | 3494.97 | 3495.0 ± 1.0 |
| N24W | CGSGSDGGVCPKILQRCRRDSDCPGACICRGWGY | 3553.05 | 3552.7 ± 1.2 |
| G25P | CGSGSDGGVCPKILQRCRRDSDCPGACICRGNPY | 3521.01 | 3521.0 ± 1.4 |
| Y26A | CGSGSDGGVCPKILQRCRRDSDCPGACICRGNGA | 3388.44 | 3388.3 ± 1.5 |
| Y26W | CGSGSDGGVCPKILQRCRRDSDCPGACICRGNGW | 3503.98 | 3503.3 ± 1.2 |

### General Materials and Methods

Analytical HPLC was performed on a HP1100 series instrument with 220 nm and 280 nm detection using a Vydac C18 column (5 mm, 4.6×150 mm) at a flow rate of 1 mL/min. Semipreparative HPLC was performed on a Waters Delta Prep system fitted with a Waters 2487 Ultraviolet-Visible (UV-vis) detector using a Vydac C18 column (15-20 μm, 10×250 mm) at a flow rate of 5 mL/min. All runs used linear gradients of 0.1% aqueous trifluoroacetic acid (TFA, solvent A) vs. 0.1% TFA, 90% acetonitrile in H₂O (solvent B). UV-vis spectroscopy was carried out on an Agilent 8453 diode array spectrophotometer, and fluorescence analysis on a Jobin Yvon Fluorolog-3 spectrofluorometer. Electrospray mass spectrometry (ES-MS) analysis was routinely applied to all cyclized peptides. ES-MS was performed on a Sciex API-150EX single quadrupole electrospray mass spectrometer, MS/MS was performed on an Applied Biosystems API 3000 triple quadrupole mass spectrometer. Calculated masses were obtained by using ProMac v1.5.3. Protein samples were analyzed by SDS-PAGE. Samples were run on Invitrogen (Carlsbad, Calif.) 4-20% Tris-Glycine Gels. The gels were then stained with Pierce (Rockford, Ill.) Gelcode Blue, photographed/digitized using a Kodak (Rochester, N.Y.) EDAS 290, and quantified using NIH Image-J software (available at the web address: rsb.info.nih.gov/ij/, last accessed on Jun. 21, 2010). DNA sequencing was performed by Davis Sequencing (Davis, Calif.) or DNA Sequencing and Genetic Analysis Core Facility at the Unviersity of Southern California using an ABI 3730 DNA sequencer, and the sequence data was analyzed with DNAStar (Madison, Wis.) Lasergene v5.5.2. All chemicals were obtained from Sigma-Aldrich (Milwaukee, Wis.) unless otherwise indicated.

### Construction of Expression Plasmids

Plasmids expressing the MCoTI-I precursors were constructed using the pTXB1 expression plasmids (New England Biolabs), which contain an engineered Mxe Gyrase intein, respectively, and a chitin-binding domain (CBD). Oligonucleotides coding for the MCoTI-I wild type and mutant sequences (Table 4) were synthesized, phosphorylated and PAGE purified by IDT DNA (Coralville, Iowa). Complementary strands were annealed in 0.3 M NaCl and the resulting double stranded DNA (dsDNA) was purified using Qiagen's (Valencia, Calif.) miniprep column and buffer PN. pTXB1 plasmids was double digested with NdeI and SapI (NEB). The linearized vectors and the MCoTI-I encoding dsDNA fragments were ligated at 15° C. overnight using T4 DNA Ligase (New England Biolabs). The ligated plasmids were transformed into DH5α cells (Invitrogen) and plated on Luria Broth (LB)-agar containing ampicillin. Positive colonies were grown in 5 mL LB containing ampicillin at 37° C. overnight and the corresponding plasmids purified using a Miniprep Kit (Qiagen). Plasmids were initially screened by EcoRI digestion, as this restriction site is removed during cloning. Preliminary positives were expressed (see below) and fully characterized by ES-MS.

### Expression and Purification of Recombinant Proteins

Origami(DE3) or Origami2(DE3) cells (Novagen, San Diego, Calif.) were transformed with the MCoTI-I plasmids (see above). Expression was carried out in LB medium (1-2 L) containing ampicillin at room temperature or 30° C. for 2 h or overnight (20 h), respectively. Briefly, 5 mL of an overnight starter culture derived from either a single clone or single plate (Ala-scan library) were used to inoculate 1 L of LB media. Cells were grown to an OD at 600 nm of about 0.5 at 37° C., and expression was induced by the addition of isopropyl-β-D-thiogalactopyranoside (IPTG) to a final concentration of 0.3 mM at the temperatures and times mentioned above. The cells were then harvested by centrifugation. For fusion protein purification, the cells were resuspended in 30 mL of lysis buffer (0.1 mM EDTA, 1 mM PMSF, 50 mM sodium phosphate, 250 mM NaCl buffer at pH 7.2 containing 5% glycerol) and lysed by sonication. The lysate was clarified by centrifugation at 15,000 rpm in a Sorval SS-34 rotor for 30 min. The clarified supernatant was incubated with chitin-beads (2 mL beads/L cells, New England Biolabs), previously equilibrated with column buffer (0.1 mM EDTA, 50 mM sodium phosphate, 250 mM NaCl buffer at pH 7.2) at 4° C. for 1 h with gentle rocking The beads were extensively washed with 50 bead-volumes of column buffer containing 0.1% Triton X100 and then rinsed and equilibrated with 50 bead-volumes of column buffer. In vivo cleavage was quantified by SDS-PAGE analysis of the purified fusion proteins using the NIH Image-J software package.

Concomitant Cleavage, Cyclization and Folding of MCoTI-I Cyclotides with GSH.

Purified MCoTI-Intein-CBD fusion proteins were cleaved with 50 mM GSH in degassed column buffer. The cyclization/folding reactions were kept for up to 2 days at 25° C. with gentle rocking. For small scale reactions, aliquots was taken each day (when necessary) and analyzed by HPLC. The reduced and oxidized circular MCoTI-I cyclotides were analyzed by ES-MS (Table 5). The supernatant of the cyclization reaction was separated by filtration and the beads were washed with additional column buffer (1 column volume per each mL of beads). The supernatant and washes were pooled, and the oxidized-circular peptides were typically purified by semipreparative HPLC using a linear gradient of 15-45% solvent B over 30 min.

### Purification of MCoTI-I Based Libraries Using Trypsin-Sepharose Beads

Preparation of trypsin-sepharose beads: NHS-activated Sepharose was washed with 15 volumes of ice-cold 1 mM HCl. Each volume of beads was incubated with an equal volume of coupling buffer (50 mM NaCl, 200 mM sodium phosphate buffer at pH 6.0) containing 2 mg of Porcine Pancreas Trypsin type IX-S (14,000 units/mg) for 3 h with gentle rocking at room temperature. The beads were then rinsed with 10 volumes of coupling buffer, and incubated with excess coupling buffer containing 100 mM ethanolamine (Eastman Kodak) for 3 h with gentle rocking at room temperature. Finally, the beads were washed with 50 volumes of wash buffer (200 mM sodium acetate buffer at pH 3, 250 mM NaCl) and stored in one volume of wash buffer.

About 30 mL of clarified lysates (in vivo obtained libraries) or 10 mL of GSH-induced cyclization/folding reaction mixture (in vitro obtained libraries) were typically incubated with 1.0 mL of trypsin-sepharose for one hour at room temperature with gentle rocking, and centrifuged at 3000 rpm for 1 min. The beads were washed with 50 volumes of PBS containing 0.1% Triton X100, then rinsed with 50 volumes of PBS, and drained of excess PBS. Bound peptides were eluted with 2.0 mL of 8 M GdmHCl and fractions were analyzed by RP-HPLC and ES-MS/MS.

### Competing Trypsin-Binding Experiments

Competing binding experiments were performed as described above but using 0.2 mL of trypsin-sepharose beads instead. For every library sample, up to 6 sequential extractions were performed. All competing trypsin-binding experiments were performed by duplicate.

### Recombinant Expression of ¹⁵N-Labeled MCoTI-I wt and K4A

¹⁵N-labeled cyclotides were produced by GSH-induced cleavage of the intein precursors in vitro as described above. Expression of intein precursors was accomplished as described above but growing the cells in M9 minimal medium containing 0.1% ¹⁵NH₄Cl. Folded cyclotides were purified by semipreparative HPLC using a linear gradient of 15-45% solvent B over 30 min. The isolated yield for both purified MCoTI cyclotides was around 0.3 mg/L. Purified products were characterized by HPLC and ES-MS (Tables 5 and 6) and 2D-NMR (FIG. 24). Total yield was about 0.5 mg of folded cyclotide per liter of bacterial culture.

### NMR Characterization of MCoTI-I wt and K4A Mutant

NMR samples were prepared by dissolving either [U-, ¹⁵N] MCoTI-I or [U-, ¹⁵N] K4A MCoTI-I into 90% H₂O/10% ²H₂O (v/v) or 100% D₂O to a concentration of approximately 0.2 mM with the pH adjusted to 3.4 by addition of dilute HCl. All ¹H NMR data were recorded on Bruker Avance II 700 MHz spectrometer equipped with a cryoprobe. Data were acquired at 27° C., and 2,2-dimethyl-2-silapentane-5-sulfonate, DSS, was used as an internal reference. All 3D experiments, ¹H{¹⁵N}-TOCSY-HSQC and ¹H{¹⁵N}-NOESY, were performed according to standard procedures (Cavanagh & Rance (1992) J. Magn. Res. 96:670-678) with spectral widths of 12 ppm in proton dimensions and 35 ppm in nitrogen dimension. The carrier frequency was centered on the water signal, and the solvent was suppressed by using WATERGATE pulse sequence. TOCSY (spin lock time 80 ms) and NOESY (mixing time 150 ms) spectra were collected using 1024 t₃ points, 256 t₂ anf 128 t₁ blocks of 16 transients.

Spectra were processed using Topspin 1.3 (Bruker). Each 3D-data set was apodized by 90°-shifted sinebell-squared in all dimensions, and zero filled to 1024×512×256 points prior to Fourier transformation. Assignments for the backbone nitrogens, H^{a} and H′ protons of folded MCoTI-I wt and mutant K4A were obtained using standard procedures. Cavanagh & Rance (1992) J. Magn. Res.96:670-678; Wuthrich (1986) NMR of Proteins and Nucleic Acids.

TABLE 4
Forward (p5) and reverse (p3) 5′-phosphorylated oligonucleotides
used to clone the different MCoTI-1-intein linear
precursors into the pTXB1 expression plasmid.

| Cyclotide | | |
|---|---|---|
| name | | Oligonucleotide sequence |
| wt | p5 | 5′-TATGtgcggttctggttctgacggtggtgtttgcccgaaaatcctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcaggattttcgggcaaacaccaccgtcagaaccagaaccgcaCA-3′ |
| V1A | p5 | 5′-TATGtgcggttctggttctgacggtggtgcttgcccgaaaatcctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcaggattttcgggcaagcaccaccgtcagaaccagaaccgcaCA-3′ |
| V1S | p5 | 5′-TATGtgcggttctggttctgacggtggttcttgcccgaaaatcctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcaggattttcgggcaagaaccaccgtcagaaccagaaccgcaCA-3′ |
| P3A | p5 | 5′-TATGtgcggttctggttctgacggtggtgtttgcgctaaaatcctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcaggattttagcgcaaacaccaccgtcagaaccagaaccgcaCA-3′ |
| K4A | p5 | 5′-TATGtgcggttctggttctgacggtggtgtttgcccggctatcctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcaggatagccgggcaaacaccaccgtcagaaccagaaccgcaCA-3′ |
| I5T | p5 | 5′-TATGtgcggttctggttctgacggtggtgtttgcccgaaaaccctgcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgcagggttttcgggcaaacaccaccgtcagaaccagaaccgcaCA-3′ |
| L6A | p5 | 5′-TATGtgcggttctggttctgacggtggtgtttgcccgaaaatcgctcagcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgctgagcgattttcgggcaaacaccaccgtcagaaccagaaccgcaCA-3′ |
| Q7G | p5 | 5′-TATGtgcggttctggttctgacggtggtgtttgcccgaaaatcctgggtcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgacccaggattttcgggcaaacaccaccgtcagaaccagaaccgcaCA-3′ |
| Q7M | p5 | 5′-TATGtgcggttctggttctgacggtggtgtttgcccgaaaatcctgatgcgttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aacgcatcaggattttcgggcaaacaccaccgtcagaaccagaaccgcaCA-3′ |
| R8A | p5 | 5′-TATGtgcggttctggttctgacggtggtgtttgcccgaaaatcctgcaggcttgcc |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgacggc |
| | | aagcctgcaggattttcgggcaaacaccaccgtcagaaccagaaccgcaCA-3′ |
| R10A | p5 | 5′-TATGtgcggttctggttctgacggtggtgtttgcccgaaaatcctgcagcgttgcg |
| | | ctcgtgactctgactgcccgggtgcttgcatctgccgtggtaacggttac-3′ |
| | p3 | 5′-GCAgtaaccgttaccacggcagatgcaagcacccgggcagtcagagtcacgagcgc |
| | | aacgctgcaggattttcgggcaaacaccaccgtcagaaccagaaccgcaCA-3′ |
| R10G | p5 | 5′-TATGtgcggttctggttctgacggtggtgtttgcccgaaaatcctgcagcgttgcg |
| | | gtcgtgactctgactgcccgggtgcttgcatctgccgtggtaacggttac-3′ |
| | p3 | 5′-GCAgtaaccgttaccacggcagatgcaagcacccgggcagtcagagtcacgaccgc |
| | | aacgctgcaggattttcgggcaaacaccaccgtcagaaccagaaccgcaCA-3′ |
| R11V | p5 | 5′-TATGtgcggttctggttctgacggtggtgtttgcccgaaaatcctgcagcgttgcc |
| | | gtgttgactctgactgcccgggtgcttgcatctgccgtggtaacggttac-3′ |
| | p3 | 5′-GCAgtaaccgttaccacggcagatgcaagcacccgggcagtcagagtcaacacggc |
| | | aacgctgcaggattttcgggcaaacaccaccgtcagaaccagaaccgcaCA-3′ |
| D12A | p5: | 5′-TATGtgcggttctggttctgacggtggtgtttgcccgaaaatcctgcagcgttgcc |
| | | gtcgtgcttctgactgcccgggtgcttgcatctgccgtggtaacggttac-3′ |
| | p3: | 5′-GCAgtaaccgttaccacggcagatgcaagcacccgggcagtcagaagcacgacggc |
| | | aacgctgcaggattttcgggcaaacaccaccgtcagaaccagaaccgcaCA-3′ |
| S13A | p5: | 5′-TATGtgcggttctggttctgacggtggtgtttgcccgaaaatcctgcagcgttgcc |
| | | gtcgtgacgctgactgcccgggtgcttgcatctgccgtggtaacggttac-3′ |
| | p3: | 5′-GCAgtaaccgttaccacggcagatgcaagcacccgggcagtcagcgtcacgacggc |
| | | aacgctgcaggattttcgggcaaacaccaccgtcagaaccagaaccgcaCA-3′ |
| D14A | p5: | 5′-TATGtgcggttctggttctgacggtggtgtttgcccgaaaatcctgcagcgttgcc |
| | | gtcgtgactctgcttgcccgggtgcttgcatctgccgtggtaacggttac-3′ |
| | p3: | 5′-GCAgtaaccgttaccacggcagatgcaagcacccgggcaagcagagtcacgacggc |
| | | aacgctgcaggattttcgggcaaacaccaccgtcagaaccagaaccgcaCA-3′ |
| P16A | p5: | 5′-TATGtgcggttctggttctgacggtggtgtttgcccgaaaatcctgcagcgttgcc |
| | | gtcgtgactctgactgcgctggtgcttgcatctgccgtggtaacggttac-3′ |
| | p3: | 5′-GCAgtaaccgttaccacggcagatgcaagcaccagcgcagtcagagtcacgacggc |
| | | aacgctgcaggattttcgggcaaacaccaccgtcagaaccagaaccgcaCA-3′ |
| G17A | p5: | 5′-TATGtgcggttctggttctgacggtggtgtttgcccgaaaatcctgcagcgttgcc |
| | | gtcgtgactctgactgcccggctgcttgcatctgccgtggtaacggttac-3′ |
| | p3: | 5′-GCAgtaaccgttaccacggcagatgcaagcagccgggcagtcagagtcacgacggc |
| | | aacgctgcaggattttcgggcaaacaccaccgtcagaaccagaaccgcaCA-3′ |
| A18G | p5: | 5′-TATGtgcggttctggttctgacggtggtgtttgcccgaaaatcctgcagcgttgcc |
| | | gtcgtgactctgactgcccgggtggttgcatctgccgtggtaacggttac-3′ |
| | p3: | 5′-GCAgtaaccgttaccacggcagatgcaaccacccgggcagtcagagtcacgacggc |
| | | aacgctgcaggattttcgggcaaacaccaccgtcagaaccagaaccgcaCA-3′ |
| I20G | p5: | 5′-TATGtgcggttctggttctgacggtggtgtttgcccgaaaatcctgcagcgttgcc |
| | | gtcgtgactctgactgcccgggtgcttgcggttgccgtggtaacggttac-3′ |
| | p3: | 5′-GCAgtaaccgttaccacggcaaccgcaagcacccgggcagtcagagtcacgacggc |
| | | aacgctgcaggattttcgggcaaacaccaccgtcagaaccagaaccgcaCA-3′ |
| R22W | p5: | 5′-TATGtgcggttctggttctgacggtggtgtttgcccgaaaatcctgcagcgttgcc |
| | | gtcgtgactctgactgcccgggtgcttgcatctgctggggtaacggttac-3′ |
| | p3: | 5′-GCAgtaaccgttaccccagcagatgcaagcacccgggcagtcagagtcacgacggc |
| | | aacgctgcaggattttcgggcaaacaccaccgtcagaaccagaaccgcaCA-3′ |
| G23A | p5: | 5′-TATGtgcggttctggttctgacggtggtgtttgcccgaaaatcctgcagcgttgcc |
| | | gtcgtgactctgactgcccgggtgcttgcatctgccgtgctaacggttac-3′ |
| | p3: | 5′-GCAgtaaccgttagcacggcagatgcaagcacccgggcagtcagagtcacgacggc |
| | | aacgctgcaggattttcgggcaaacaccaccgtcagaaccagaaccgcaCA-3′ |
| N24W | p5: | 5′-TATGtgcggttctggttctgacggtggtgtttgcccgaaaatcctgcagcgttgcc |
| | | gtcgtgactctgactgcccgggtgcttgcatctgccgtggttggggttac-3′ |
| | p3: | 5′-GCAgtaaccccaaccacggcagatgcaagcacccgggcagtcagagtcacgacggc |
| | | aacgctgcaggattttcgggcaaacaccaccgtcagaaccagaaccgcaCA-3′ |
| G25P | p5: | 5′-TATGtgcggttctggttctgacggtggtgtttgcccgaaaatcctgcagcgttgcc |
| | | gtcgtgactctgactgcccgggtgcttgcatctgccgtggtaacccgtac-3′ |
| | p3: | 5′-GCAgtacgggttaccacggcagatgcaagcacccgggcagtcagagtcacgacggc |
| | | aacgctgcaggattttcgggcaaacaccaccgtcagaaccagaaccgcaCA-3′ |
| Y26W | p5: | 5′-TATGtgcggttctggttctgacggtggtgtttgcccgaaaatcctgcagcgttgcc |
| | | gtcgtgactctgactgcccgggtgcttgcatctgccgtggtaacggttgg-3′ |
| | p3: | 5′-GCAccaaccgttaccacggcagatgcaagcacccgggcagtcagagtcacgacggc |
| | | aacgctgcaggattttcgggcaaacaccaccgtcagaaccagaaccgcaCA-3′ |
| Y26A | p5: | 5′-TATGtgcggttctggttctgacggtggtgtttgcccgaaaatcctgcagcgttgcc |
| | | gtcgtgactctgactgcccgggtgcttgcatctgccgtggtaacggtgct-3′ |
| | p3: | 5′-GCAagcaccgttaccacggcagatgcaagcacccgggcagtcagagtcacgacggc |
| | | aacgctgcaggattttcgggcaaacaccaccgtcagaaccagaaccgcaCA-3′ |

TABLE 5
Summary of the ¹H and ¹⁵N^{a} NMR assignments for the main-chain
protons (i.e. N^{a}-H and C^{a}-H) of recombinant MCoTI-I.

| Residues | ¹H-¹⁵N^{a} (ppm) | ¹⁵N^{a} (ppm) | ¹H-C^{a} (ppm) |
|---|---|---|---|
| Val 1 | 8.33 | 120.96 | 3.89 |
| Cys 2 | 8.54 | 126.39 | 4.92 |
| Pro 3 | | | |
| Lys 4 | 8.10 | 120.61 | 4.17 |
| Ile 5 | 7.523 | 119.968 | 4.25 |
| Lue 6 | 8.53 | 125.78 | 4.35 |
| Gln 7 | | | |
| Arg 8 | 8.61 | 127.62 | 4.37 |
| Cys 9 | 8.34 | 120.34 | 4.49 |
| Arg 10 | 7.97 | 117.20 | 4.58 |
| Arg 11 | 9.36 | 117.83 | 4.35 |
| Asp 12 | 9.32 | 120.98 | 4.76 |
| Ser 13 | 8.27 | 115.75 | 4.68 |
| Asp 14 | 7.65 | 120.57 | 4.44 |
| Cys 15 | 7.97 | 117.74 | 4.82 |
| Pro 16 | | | |
| Gly 17 | 8.37 | 106.63 | 3.65 |
| Ala 18 | 8.33 | 125.14 | 4.31 |
| Cys 19 | 8.06 | 117.00 | 4.50 |
| Ile 20 | 8.9 | 113.42 | 4.24 |
| Cys 21 | 9.03 | 124.20 | 3.95 |
| Arg 22 | 8.02 | 128.58 | 4.17 |
| Gly 23 | 8.79 | 108.27 | 3.80 |
| Asn 24 | 7.70 | 115.77 | 4.52 |
| Gly 25 | 8.30 | 107.30 | 3.82 |
| Tyr 26 | 7.19 | 116.66 | 5.10 |
| Cys 27 | 8.68 | 120.72 | 5.19 |
| Gly 28 | 9.72 | 109.62 | 4.36, 3.73 |
| Ser 29 | 8.71 | 115.78 | 4.34 |
| Gly 30 | 9.06 | 117.73 | 3.71, 4.22 |
| Ser 31 | 8.58 | 116.05 | 4.27 |
| Asp 32 | 8.31 | 122.07 | 4.49 |
| Gly 33 | 8.08 | 10844 | 3.67, 3.89 |
| Gly 34 | 8.05 | 110.83 | 3.67, 4.14 |

TABLE 6
Summary of the ¹H and ¹⁵N^{a} NMR assignments for the main-chain
protons (i.e. N^{a}-H and C^{a}-H) of recombinant MCoTI-I K4A.

| Residues | ¹H-¹⁵N^{a} (ppm) | ¹⁵N (ppm) | ¹H-C^{a} (ppm) |
|---|---|---|---|
| Val 1 | 8.32 | 121.17 | 3.80 |
| Cys 2 | 8.68 | 125.40 | 4.86 |
| Pro 3 | | | |
| Ala 4 | 6.70 | 124.03 | 4.44 |
| Ile 5 | 7.57 | 118.41 | 4.21 |
| Lue 6 | 8.40 | 122.79 | 4.27 |
| Gln 7 | 8.36 | 122.55 | 4.30 |
| Arg 8 | 8.51 | 127.02 | 4.30 |
| Cys 9 | 8.36 | 120.17 | |
| Arg 10 | 7.95 | 117.17 | 4.58 |
| Arg 11 | 9.34 | 117.80 | 4.27 |
| Asp 12 | 9.32 | 120.91 | 4.81 |
| Ser 13 | 8.23 | 115.57 | 4.67 |
| Asp 14 | 7.63 | 120.54 | 4.40 |
| Cys 15 | 7.96 | 117.81 | 4.77 |
| Pro 16 | | | |
| Gly 17 | 8.31 | 106.30 | 3.65 |
| Ala 18 | 8.36 | 124.53 | 4.31 |
| Cys 19 | 7.93 | 116.09 | 4.63 |
| Ile 20 | 8.88 | 113.42 | 4.19 |
| Cys 21 | 9.006 | 124.15 | 3.93 |
| Arg 22 | 8.04 | 128.53 | 4.17 |
| Gly 23 | 8.77 | 108.24 | 3.80 |
| Asn 24 | 7.65 | 115.71 | 4.64 |
| Gly 25 | 8.25 | 107.23 | 3.55, 3.89 |
| Tyr 26 | 7.14 | 116.71 | 5.10 |
| Cys 27 | 8.67 | 121.05 | 5.13 |
| Gly 28 | 9.63 | 108.99 | 3.75, 4.30 |
| Ser 29 | 8.78 | 115.94 | 4.37 |
| Gly 30 | 9.06 | 111.70 | 3.72, 4.22 |
| Ser 31 | 8.55 | 116.07 | 4.28 |
| Asp 32 | 8.29 | 122.04 | 4.49 |
| Gly 33 | 8.01 | 108.48 | 3.67 |
| Gly 34 | 8.04 | 110.76 | 4.65 |

### Experiment No. 5

### Cell-Based Fluorescence Reporter for Screening Protease Inhibitors Against Lethal Factor Protease

This example shows the construction of a cell-based fluorescent reporter for anthrax lethal factor (LF) protease activity using the principle of fluorescence resonance energy transfer (FRET). This was accomplished by engineering an Escherichia coli cell line to express a genetically encoded FRET reporter and LF protease. Both proteins were encoded in two different expression plasmids under the control of different tightly controlled inducible promoters. The FRET-based reporter was designed to contain a LF recognition sequence flanked by the FRET pair formed by CyPet and YPet fluorescent proteins. The length of the linker between both fluorescent proteins was optimized using a flexible peptide linker containing several Gly-Gly-Ser repeats. These results indicate that this FRET-based LF reporter was readily expressed in E. coli cells showing high levels of FRET in vivo in the absence of LF. The FRET signal, however, decreased 5 times after inducing LF expression in the same cell. These results demonstrate that this cell-based LF FRET reporter can be used to screen genetically encoded libraries in vivo against LF.

### Molecular Design, Expression, and Purification of Fret-Based Reporters for Lf Protease Activity

The different LF FRET reporters (FIG. 29, 1 to 5) were constructed by fusing two optimized fluorescent proteins (CyPet and YPet) (Nguyen et al. (2005) Nat. Biotechnol. 23:355-360) to a consensus LF recognition sequence (Turk et al. (2004) Nature Structural & Molecular Biology 11:60-66) (FIG. 29). The CyPet-Ypet pair was originally obtained by evolutionary optimization from cyan fluorescent protein (CFP) and yellow fluorescent protein (YFP) by Daugherty and co-workers (Nguyen et al. (2005) Nat. Biotechnol. 23:355-360). This optimized FRET pair enables intracellular FRET measurements with enhanced sensitivity and dynamic range, and thus allows the use of standard flow cytometry instrumentation for high-throughput analysis and screening applications. You et al. (2006) Proc. Natl. Acad. Sci. USA 103:18458-18463. This is critical for performing screening assays inside living cells. The LF consensus sequence was derived from a peptide obtained by Cantley and co-workers (Turk et al. (2004) Nature Structural & Molecular Biology 11:60-66) from the analysis of partially degenerated peptide libraries to provide an optimal substrate for LF protease. This peptide incorporates consensus residues (P5-P4′) surrounding the scissile bond based on the peptide library screen, flanked by residues from MAPKK2. The crystal structure of this peptide with an inactive form of LF showed that nine residues of the substrate (from P3 to P6′) bind in an extended conformation along the 40 Å-long substrate recognition groove. Turk et al. (2004) Nature Structural & Molecular Biology 11:60-66; Pannifer et al. (2001) Nature 414:229-233. Thus, in order to release any potential steric hindrance introduced by the two flanking fluorescent proteins on the peptide substrate, several FRET-based reporters (FIG. 29, 1 to 5) encoding several repeats of the flexible tripeptide Gly-Gly-Ser (Evers et al. (2006) Biochemistry 45:13183-13192) between the LF substrate and the CyPet and YPet fluorescent proteins were designed (FIG. 29).

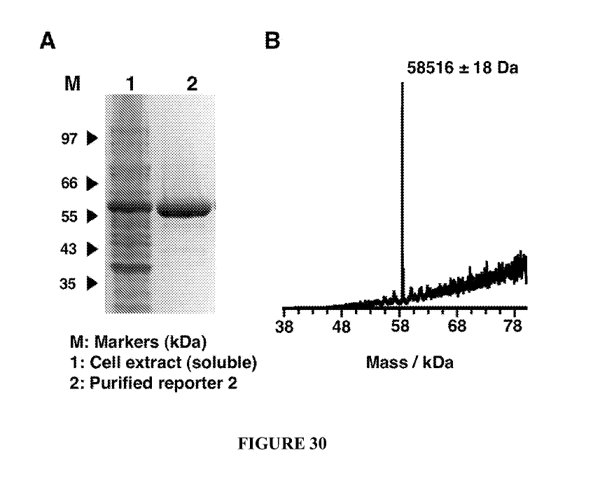

Characterization of all the genetically encoded FRET-based reporters was carried out first in vitro. For this purpose constructs 1 to 5 (FIG. 29) were first cloned onto a T7-driven. bacterial expression vector, expressed in E. coli and readily purified using Ni-NTA affinity chromatography. All the FRET reporters showed high expression levels (about 10 mg of pure protein per liter of culture), and were characterized by ES-MS and SDS-PAGE (FIGS. 29 and 30).

### Fluorescence Properties of FRET-Based Reporters 1 to 5

Protein constructs 1 to 5 were analyzed by fluorescence spectroscopy to evaluate their FRET efficiency in the “on” state. In all the cases CyPet-YPet fusion proteins showed high FRET values when excited at 414 nm. The FRET values, estimated as the ratio between the fluorescence intensities at 525 nm (maximum emission for YPet) and 475 nm (maximum emission for CyPet), ranged from 9.5 for construct 5 to 6.2 for construct 6 (see FIG. 29). In contrast, an equimolar solution of YPet and CyPet gave a FRET value of only 0.7. As expected, increasing the length of the flexible linker (Gly-Gly-Ser)ₙ decreased the FRET efficiency of the corresponding reporter proteins around 35% (construct 1 versus 6). It is interesting to note, however, the relatively high FRET values observed for constructs 4 and 5. These constructs contain 8 and 12 Gly-Gly-Ser repeats, which in a completely extended conformation have lengths of 116 Å and 78 Å, respectively. These values are well beyond the Foster radius (about 50 Å) assigned for the FRET pair formed by cyan and yellow fluorescent proteins. Evers et al. (2006) Biochemistry 45:13183-13192. Recent studies by Merkx and co-workers (Evers et al. (2006) Biochemistry 45:13183-13192; Dongen et al. (2007) J. Am. Chem. Soc.) on the effect of flexible linkers in FRET-based biosensors have shown similar results, indicating that the behavior of these flexible linkers can be better described as random coils using either a worm-like chain or Gaussian chain model rather than as a totally extended conformation.

### Expression and Enzymatic Activity of Recombinant LF Protease

The LF protease employed in this work was cloned from B. anthracis strain Sterne (Kwon et al. (2004) Org. Lett. 6:3801-3804) into a T7-driven pRSF bacterial expression plasmid. Som et al. (1982) Mol. Gen. Genet. 187:375-383 The protein was readily expressed in E. coli and purified by Ni-NTA chromatography. A total of 10 mg of purified LF protease per liter of E. coli culture could be routinely obtained. The activity of recombinantly expressed LF protease was tested using consensus peptide H-RRKKVYPYPMEGTIA-NH₂ as a substrate in a 200:1 peptide-to-enzyme ratio. The cleavage reaction was monitored by analytical RP-HPLC indicating that the protease was totally active. LF was able to cleave specifically the peptide bond between P1-P1′ residues of the peptide substrate in less than 15 minutes (data not shown) using the conditions described under Materials and Methods below.

### In Vitro Cleavage of FRET-Based Reporters 1 to 5 by LF Protease

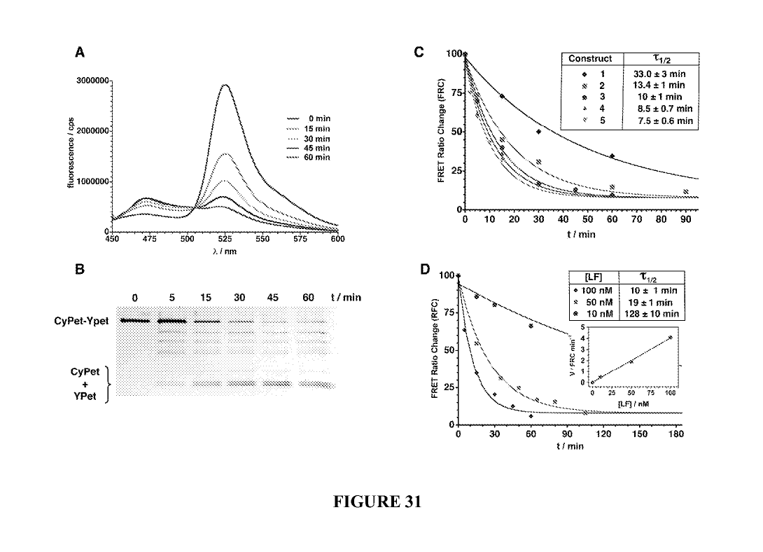

LF cleavage of FRET reporters 1 to 5 (see FIG. 29) was monitored by fluorescence spectroscopy. This was carried out by treating a 10 nM solution of the corresponding FRET reporter with different amounts of purified LF protease at 37° C. in LF reaction buffer. Under these conditions, all constructs showed between a 9- to 14-fold decrease in FRET signal upon cleavage with LF (FIG. 31). Cleavage reactions were also monitored by SDS-PAGE (FIG. 31B) confirming that the proteolytic cleavage was specifically taking place at the linker located between the two fluorescent proteins. As the cleavage reaction progressed, the initial band about 57 kDa corresponding to the FRET reporter disappeared giving rise to a doublet of bands around 28 kDa. The FRET value once the cleavage was complete was estimated to be about 0.7 in all the constructs. This value is similar to that found for an equimolar mixture of CyPet and YPet proteins.

Applicant also explored the effect of the linker length on the efficiency of LF protease cleavage. As expected, the cleavage reaction proceeds more efficiently as the length of the flexible linker Gly-Gly-Ser increases. As shown if FIG. 31C, the change in half-life time is more pronounced when Applicant compared constructs 1 to 3. In each case, cleavage occurs about twice as fast with each couple of Gly-Gly-Ser repeats added, with t_{1/2} values of 33, 17, and 9 minutes, respectively. Constructs 3 to 6, however, showed very similar half-life times under the same reaction conditions, showing only a marginal increase in the rate of cleavage (t_{1/2} values for constructs 3, 4, and 5 were 9, 8, and 7 minutes, respectively). These results confirm that the presence of two Gly-Gly-Ser repeats on each side of the LF recognition sequence is enough to release most of the steric hindrance introduced by the presence of the two large fluorescent proteins.

The cleavage of the FRET-based reporter by LF was also shown to be dose-dependent on the concentration of LF thus indicating that the cleavage was specific for the consensus sequence embedded in the FRET-based reporter protein. As shown in FIG. 31D, the initial rate of cleavage for construct 3 was shown to be proportional to the concentration of LF used in a range from 10 to 100 nM. These results show the potential of these constructs to be used as a sensitive LF probe able to detect nM concentrations of LF protease in vitro.

Design of an LF Protease FRET Reporter System in Live E. coli Cells

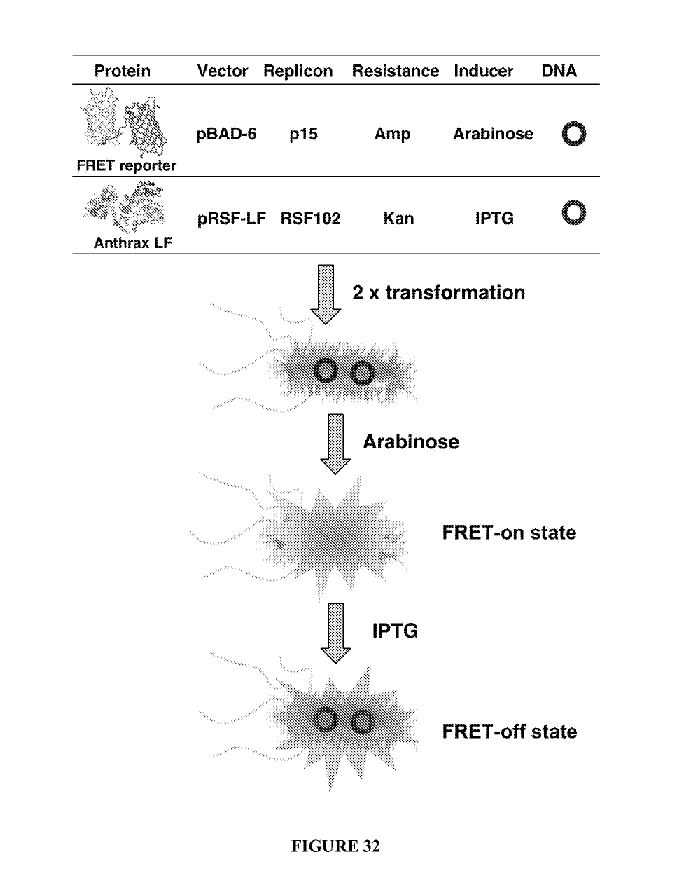

The next step was to explore the possibility of using a FRET-based reporter to work inside living E. coli cells for the in vivo screening of LF inhibitors. Key to this was the use of two orthogonal plasmids with tightly controlled inducible promoters for individual expression of LF protease and its FRET-based substrate (FIG. 32). This was accomplished using the pRSF and pBAD families of expression vectors for the selective expression of LF and its FRET-based reporter, respectively.

Based on previous results, Applicant decided to use an intracellular reporter with six Gly-Gly-Ser repeats on each side of the LF recognition sequence. This long linker allows rapid cleavage of the LF recognition sequence while still showing a relatively high dynamic range for FRET change upon cleavage. This new construct (6, see FIG. 29), which is virtually identical to 5, was cloned into an expression pBAD-derived vector to give the plasmid pBAD-6. This expression plasmid contains an araBAD-driven promoter and a p15 replicon. On the other hand, LF was cloned into a pRSF-based vector to give the expression plasmid pRSF-LF as described earlier. This expression vector contains a T7-driven promoter and an RSF origin of replication. Som (1982) Mol. Gen. Genet. 187:375-383. These two expression plasmids are fully compatible for the sequential expression of proteins in E. coli cells, and they have been used for the study of protein-protein interactions in vivo. Burz et al. (2006) Nature Methods 3:91-93.

### In Vivo Sensing of LF Activity

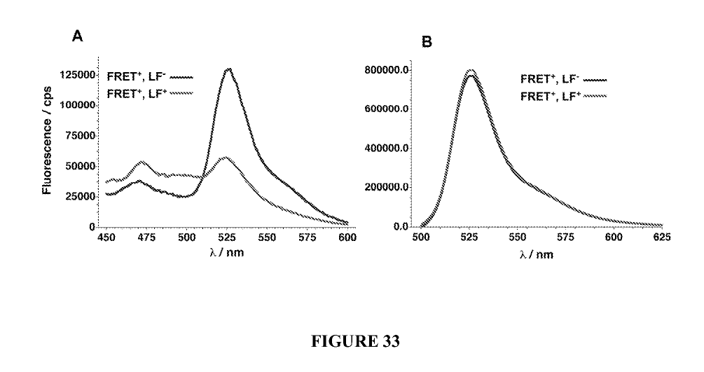

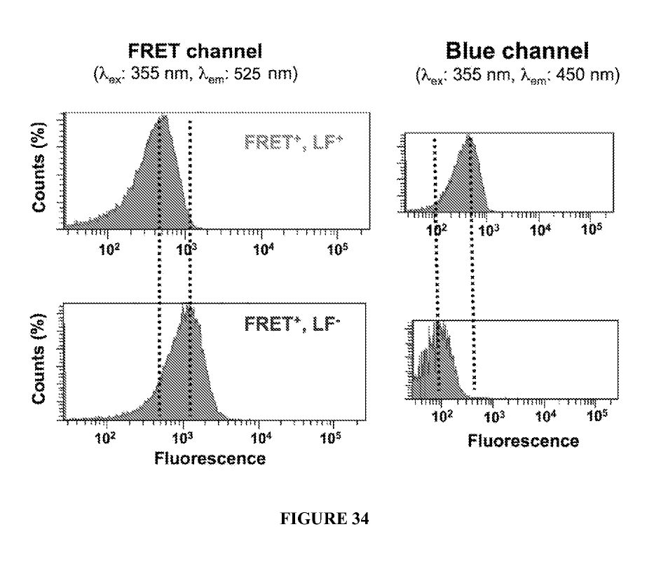

In order to explore the potential of construct 6 to optically sense LF activity inside living cells, plasmids pBAD-6 and pRSF-6 were co-transformed into E. coli BL21(DE3) cells. Induction of FRET reporter 6 was performed first by inducing the cells overnight with L-arabinose. Because the cells employed in this experiment were capable of metabolizing L-arabinose, two more aliquots of L-arabinose were added every 4 hours during the first 8 hours of induction. The cells were also supplemented with glucose to repress any residual expression of LF during this time. The presence of glucose is known to repress both araBAD and T7/lac promoters (Grossman et al. (1998) Gene 209:95-103; Boomershine et al. (2003) Protein Expr. Purif. 28:246-251), but in the presence of L-arabinose the reporter protein was expressed, albeit at a slower rate. Burz et al. (2006) Nature Methods 3:91-93; Boomershine et al. (2003) Protein Expr. Purif. 28:246-251. At this point the cells were harvested and resuspended in minimal media M9. An aliquot of these cells was analyzed by fluorescence spectroscopy (FIG. 33). The fluorescence spectrum revealed the presence of a strong FRET emission signal at 525 nm indicating the presence of reporter protein 6. The in vivo FRET value (estimated as the ratio between the fluorescence intensities at 525 nm and 475 nm) for construct 6, however, was smaller than the reported for construct 5 in vitro (3.4 versus 6.2). Intrigued by this difference, because reporters 5 and 6 share the same linker composition, Applicants decided to express reporter 6 using the same conditions as before but employing E. coli cells transformed only with pBAD-6 instead. The fluorescence analysis of the resulting cells provided a similar FRET value thus ruling out the presence of any prematurely expressed LF as the cause for the smaller FRET value observed for construct 6 in vivo. Based on these results, it is very likely that the observed decrease in FRET efficiency inside living cells could be due to a non-specific interaction between the reporter protein and some unidentified component of the cellular background.

Applicant then decided to express LF and evaluate the ability of construct 6 to sense its activity in vivo. The previously induced fluorescent cells resuspended in M9 were complemented with glycerol and the appropriate antibiotic, and then induced with IPTG at 30° C. The proteolytic reaction was monitored by taking small aliquots of cells at different times and measuring their fluorescence spectra. Within 1 hour of induction, the FRET signal decreased from a value of 3.4 (“FRET-on” state) to 0.9 (“FRET-off” state) as shown in FIG. 33. Longer induction times of up to 5 hours gave similar fluorescence spectra and FRET ratios, indicating the cleavage reaction was completed in less than 1 hour. Moreover, the FRET-off state observed in vivo was practically identical to the value observed in vitro, further confirming the total cleavage of the reporter protein in vivo.

Quantification of the YPet protein before and after LF induction indicated that the concentration of YPet remains constant and therefore no more reporter protein was expressed during the induction of LF (FIG. 33B). Furthermore, the intracellular concentration of construct 6 was estimated to be about 20 μM. At these concentration levels, the propensity of cleaved CyPet and YPet to heterodimerize, thus increasing the FRET signal, is minimal. Nguyen et al. (2005) Nat. Biotechnol. 23:355-360; You et al. (2006) Proc. Natl. Acad. Sci. USA 103:18458-18463. These results confirm that the change in FRET signal was due only to proteolytic cleavage.

These results show that E. coli cell strains co-transformed with pBAD-6 and pRSF-LF can be efficiently used for in vivo screening of libraries of compounds. Key to this approach is to maintain LF and its substrate under the control of two tightly regulated and inducible promoters. This allows us to distinguish the FRET-on state, where only the substrate is expressed, from the FRET-off estate, where LF is expressed in the presence of the substrate (FIG. 33). Hence, addition or expression, by the same cell, of any potential LF inhibitor during the FRET-on state can be readily screened by measuring the FRET ratio at different times during the induction of LF. Potential inhibitors will inhibit the cleavage of the substrate at early times of LF induction. In contrast, cells containing non-inhibitors will efficiently cleave the substrate, rapidly reaching the FRET-off state.

### Materials and Methods

### Solid-Phase Peptide Synthesis of the LF Consensus Peptide (H-RRKKVYPMEGTIA-OH)

Peptide synthesis was manually performed using the HBTU activation protocol for Fmoc solid-phase peptide synthesis on a 4-Fmoc-Rink amide resin (Novabiochem). Coupling yields were monitored by the quantitative ninhydrin determination of residual free amine. Protected N^{a}-Fmoc amino acids were purchased from Novabiochem. Side-chain protection was employed as previously described for the Fmoc protocol. Methionine was introduced as unprotected Fmoc-Met-OH.

### Construction of CyPet-YPet Fusion Protein Reporters (1 to 6)

The CyPet-YPet parent bacterial expression construct containing both optimized CyPet and YPet was engineered as follows. First, DNA encoding CyPet was obtained by PCR amplification using the pCyPet-His plasmid as a template. The DNA amplicon was then inserted into the NheI and BamHI sites of the T7 expression vector pET28a(+) (Novagen) to give plasmid pET28a-CyPet. In a second step, DNA encoding YPet was produced by PCR amplification using the pYPet-His plasmid as a template. This DNA was inserted in frame into the HindIII and XhoI sites of pET28a-CyPet to give plasmid pET28a-CyPet-YPet. The forward and reverse primers employed for the PCR amplification of the DNA encoding for CyPet are 5′-GGC CAG GAG TGC TAG CAT GTC TAA AGG TG-3′ and 5′-GGT GGT GGT GGG ATC CTT TGT ACA ATT CAT CC-3′, respectively. The forward and reverse primers employed for PCR amplification of the DNA encoding for YPet are 5′-CAC TAA GGC CAG GAA AGC TTC GAT GTC TAA AGG-3′ and 5′-CCT TAG TGG TGG TGC TCG AGT TAT TTG TAC AAT-3′, respectively. In all the cases, the resulting PCR amplicons and plasmids were digested with their respective restriction endonucleases (NEB) and purified by gel using the QIAquick Gel Extraction Kit (Qiagen) prior to ligation with T4 DNA ligase (NEB). The DNA encoding the LF substrate consensus sequence was cloned into the plasmid pET28a-CyPet-YPet using the BamHI and HindIII sites. Different inserts encoding the LF substrate flanked by various repeats of the flexible tripeptide GGS were prepared (FIG. 29). Constructs 1, 2, 3, 4, and 5 encoded 0, 1, 2, 4, and 6 pairs of the tripeptide GGS, respectively, and were prepared as follows. 5′-Phosphorylated top (p5) and bottom (p3) strand oligonucleotides were synthesized by IDT DNA (Coralville, Iowa). Complementary strands were annealed in 20 mM sodium phosphate, 0.3 M NaCl buffer at pH 7.4 and the resulting double-stranded DNA (dsDNA) was purified using QIAquick PCR Purification Kit (Qiagen). In plasmids encoding constructs 4 and 5, the dsDNA inserts were constructed by first ligating the dsDNA resulting from annealing oligonucleotides p5a-p3a and p5b-p3b (see Table 7). The resulting dsDNA was 5′-phosphorylated with T4 PNK (NEB). This strategy was employed due to the decreased yield and purity associated with synthetic oligonucleotides larger than 100 bases. All the dsDNA inserts were introduced in frame into the BamHI and XhoIII sites of pET28a-CyPet-Ypet to give plasmids pET28a-1 to pET28a-5.

Reporter protein construct 6 is similar to construct 5 but it was cloned into the expression vector pBAD/Myc-HisA (Invitrogen) using the SacI and KpnI restriction sites to give plasmid pBAD-6. Forward and reverse primers containing SacI and KpnI, respectively, were used to amplify by PCR the DNA encoding construct 5 using pET28a-5 as a template. The resulting amplicon was inserted into the SacI and KpnI sites of the expression vector pBAD/Myc-HisA. The forward and reverse primer sequences used in the PCR amplification are 5′-ATA TAT GAG CTC TAG CAT GTC TAA AGG TGA AGA-3′ and 5′-AAT ATA GGT ACC TTG TAC AAT TCA TTC ATA CCC-3′, respectively.

All plasmids were first transformed into competent E. coli DH5a cells (Invitrogen) and plated on Luria broth (LB)-agar containing either kanamycin (34 mg/L) for the pET28a-derived vectors or ampicillin (100 mg/L) for the pBAD-derived vector. Positive colonies were grown in 5 mL of LB containing the appropriate antibiotic at 37° C. overnight and the corresponding plasmid purified using a miniprep kit (Qiagen). Plasmids were either screened by PCR using the same cloning primers or by digestion using the same restriction endonucleases used for cloning. Positive plasmids were sequenced and screened for bacterial expression.

Expression and Purification of CyPet-YPet Fusion Protein Reporters (1 to 5). E. coli

Rosetta 2(DE3) cells (Novagen) were transformed with plasmids pET28a-1 to pET28a-5. Expression was carried out in 1 L of LB medium containing ampicillin (100 mg/L) and chloramphenicol (34 mg/L) at 20° C. overnight. Briefly, 5 mL of an overnight starter culture derived from a single clone was used to inoculate 1 L of LB media. Cells were grown to an OD at 600 nm of about 0.5 at 37° C., and expression was induced by the addition of isopropyl-b-D-thiogalactosidase (IPTG) to a final concentration of 0.25 mM at 20° C. overnight. The cells were harvested by centrifugation, resuspended in 30 mL of lysis buffer (0.1 mM PMSF, 25 mM sodium phosphate, 150 mM NaCl buffer at pH 8.0 containing 5% glycerol) and lysed by sonication. The lysate was clarified by centrifugation at 15,000 rpm in a Sorval SS-34 rotor for 30 minutes. The clarified supernatant was incubated with 1 mL of Ni-NTA agarose beads (Qiagen) previously equilibrated with column buffer (25 mM sodium phosphate, 150 mM NaCl buffer at pH 8.0) at 4° C. for 1 hour with gentle rocking. The Ni-NTA agarose beads were washed sequentially with column buffer containing 10 mM imidazole (100 mL) followed by column buffer containing 20 mM imidazole (100 mL). The fusion protein was eluted with 2 mL of column buffer containing 100 mM EDTA. Proteins were characterized as the desired product by ES-MS (see FIG. 29). Quantification of the CyPet-YPet fusion proteins was carried out spectrophotometrically using an extinction coefficient per chain at 517 nm of 104,000 M⁻¹ cm⁻¹. Approximately 10 mg of FRET reporter protein were purified per 1 L of culture grown as indicated above.

Construction of LF Protease Expression Vector (pRSF-LF)

The DNA encoding LF protease from B. anthracis strain Sterne was isolated by PCR using the pTXB1-LF plasmid as a template. The forward (5′-TAA GGA TCC GGC GGG CGG TCA TGG TGA-3′) and reverse (5′-GCA TCT CCC GTG ATG CAG GAA-3′) primers contained a BamHI and NotI restriction site, respectively. The resulting amplicon was purified using Qiagen's PCR purification kit, digested and ligated into BamHI- and NotI-treated plasmid pRSFDuet-1 (Novagen) to give T7 expression vector pRSF-LF. The resulting plasmid was sequenced and shown to be free of mutations.

Expression and Purification of LF Protease. E. coli

BL21(DE3) cells (1 L) transformed with pRSF-LF plasmid were grown to mid-log phase (OD₆₀₀≈0.5) in LB medium containing kanamycin (34 mg/L) and induced with 0.15 mM IPTG at 22° C. for 16 hours. The cells were pelleted by centrifugation, resuspended in 30 mL of lysis buffer and lysed by sonication. The lysate was clarified by centrifugation and purified on Ni-NTA agarose beads as described above. The protein was eluted with column buffer containing 100 mM EDTA and dialyzed against LF reaction buffer (10 μM CaCl₂, 100 μM ZnCl₂, 10 mM MgCl₂, 20 mM NaPi, 100 NaCl buffer at pH 7.2). The purified protein was quantified by UV spectroscopy using an extinction coefficient per chain at 280 nm of 79,650 M⁻¹ cm⁻¹, and characterized by SDS-PAGE and ES-MS (calculated molecular weight: 92949.0 Da, observed mass: 92980±20 Da). The enzymatic activity of the LF protease was tested against the consensus peptide and the different FRET reporter constructs.

### In Vitro LF Proteolytic Assay Using Hplc

An HPLC-based LF proteolytic assay was performed by incubating the consensus peptide (200 μM) with LF (10 μM) in 500 μL of LF reaction buffer at 37° C. for 30 minutes and analyzed by HPLC and ES-MS.

### In-Vitro FRET-Based LF Proteolytic Assay

The enzymatic assay was performed by incubating the different FRET reporter constructs (1 to 5, 10 nM) with purified LF protease (10 nM) in 3 mL of LF reaction buffer in a quartz cuvette. The reaction was kept with gentle stirring at 37° C. for up to 180 minutes. Reaction progress was continuously monitored by fluorimetry using a Spex FluoroLog-3 spectrofluorometer with both excitation and emission slits set at 5 nm. For FRET measurements, the excitation wavelength was set to 414 nm and fluorescence scans were carried out at a rate of 2 nm/s from 450 nm to 600 nm. The relative FRET ratio change was calculated as previously reported using: FRC=[(I₀⁵²⁵/I₀⁴⁷⁵)/(Iₜ^{525/I}ₜ⁴⁷⁵)], where I₀ and Iₜ are the fluorescence intensities at time zero and at particular time (t), respectively, either at 525 nm or 475 nm.

Sequential Expression of FRET Reporter 6 and LF Protease. E. coli

BL21(DE3) cells were transformed sequentially with plasmid pBAD-6 and pRSF-LF. The resulting cells were grown at 37° C. to mid-log phase (OD₆₀₀≈0.5) in LB media containing ampicillin (100 mg/L) and kanamycin (34 mg/L), and supplemented with 0.5% glucose. Expression of FRET-based protein reporter 6 was induced by adding 1/100th culture volume of 20% w/v of L-arabinose for 8 hours at 30° C. During this period, an aliquot of L-arabinose was added every 4 hours. After the induction period, cells were incubated at 22° C. overnight and then pelleted by centrifugation. The cells were washed once and then resuspended in minimal media M9 containing glycerol (4 mL/L) as the sole carbon source, then supplemented with ampicillin (100 mg/L) and kanamycin (34 mg/L). Prior to the induction with IPTG, a 10 mL sample of cells was taken for fluorescence analysis. The rest of the cells were induced with 0.25 mM IPTG at 30° C. Small aliquots of cells were taken at different time points for fluorescence analysis.

### In Vivo FRET-Based Analysis of LF Protease Activity

Cell aliquots (1 mL) were briefly centrifuged at 5,000 rpm for 30 seconds, washed three times with 1 mL of PBS (20 mM NaPᵢ, 100 mM NaCl buffer at pH 7.2), and resuspended in 1 mL of PBS. The concentration of cells was finally adjusted to an OD₆₀₀ of 0.6 with PBS. Cell samples (100 μL) were mixed with 2.9 mL of PBS in a quartz cuvette kept at 37° C. The samples were gently stirred with a magnetic stir bar. Fluorescence analysis was performed as described in the in vitro experiments (see above). Protein concentrations were quantified by fluorescence using excitation and emission wavelengths of 490 nm and 525 nm, respectively, and then compared against the 525 nm emission of purified FRET reporter of a known concentration previously calculated by UV-vis.

TABLE 7
Oligonucleotides used to encode different LF recognition sites
containing a variable number of (Gly-Gly-Ser)ₙ repeats.

Construct 1
p5: 5′-GAT CCC GTC GTA AAA AAG TTT ATC CGT ATC CGA TGG AAG GTA CCA TCG CCC
A-3′
p3: 5′-AGC TTG GGC GAT GGT ACC TTC CAT CGG ATA CGG ATA AAC TTT TTT ACG ACG
G-3′
Construct 2
p5: 5′-GAT CCG GTG GCA GCC GTC GTA AAA AAG TTT ATC CGT ATC CGA TGG AAC CGA
CCA TCG CCG GTG GCA GCC A-3′
p3: 5′-AGC TTG GCT GCC ACC GGC GAT GGT CGG TTC CAT CGG ATA CGG ATA AAC TTT
TTT ACG ACG GCT GCC ACC G-3′
Construct: 3
p5: 5′-GAT CCG GTG GCA GCG GTG GCA GCC GTC GTA AAA AAG TTT ATC CGT ATC CGA
TGG AAC CGA CCA TCG CCG GTG GCA GCG GTG GCA GCC A-3′
p3: 5′-AGC TTG GCT GCC ACC GCT GCC ACC GGC GAT GGT CGG TTC CAT CGG ATA CGG
ATA AAC TTT TTT ACG ACG GCT GCC ACC GCT GCC ACC G-3′
Construct: 4
p5a: 5′-GAT CCG GTG GCA GCG GTG GCA GCG GTG GCA GCG GTG GCA GCC GTC GTA AAA
AAG TTT ATC C-3′
p3a:: 5′-GAT ACG GAT AAA CTT TTT TAC GAC GGC TGC CAC CGC TGC CAC CGC TGC CAC
CGC TGC CAC CG-3′
p5b: 5′-GTA TCC GAT GGA ACC GAC CAT CGC CGG TGG CAG CGG TGG CAG CGG TGG
CAG CGG TGG CAG CCA-3′
p3b: 5′-AGC TTG GCT GCC ACC GCT GCC ACC GCT GCC ACC GCT GCC ACC GGC GAT GGT
CGG TTC CAT CG-3′
Construct: 5
p5a: 5′-GAT CCG GTG GCA GCG GTG GCA GCG GTG GCA GCG GTG GCA GCG GTG GCA
GCG GTG GCA GCC GTC GTA AAA AAG TTT ATC C-3′
p3a: 5′-GAT ACG GAT AAA CTT TTT TAC GAC GGC TGC CAC CGC TGC CAC CGC TGC CAC
CGC TGC CAC CGC TGC CAC CGC TGC CAC CG-3′
p5b: 5′-GTA TCC GAT GGA ACC GAC CAT CGC CGG TGG CAG CGG TGG CAG CGG TGG
CAG CGG TGG CAG CGG TGG CAG CGG TGG CAG CCA-3′
p3b: 5′-AGC TTG GCT GCC ACC GCT GCC ACC GCT GCC ACC GCT GCC ACC GCT GCC ACC
GCT GCC ACC GGC GAT GGT CGG TTC CAT CG-3′

### Experiment No. 6

### Cloning, Expression and Characterization of a Combinatorial Library Based on Loop 2 of MCoTI-I IN E. coli.

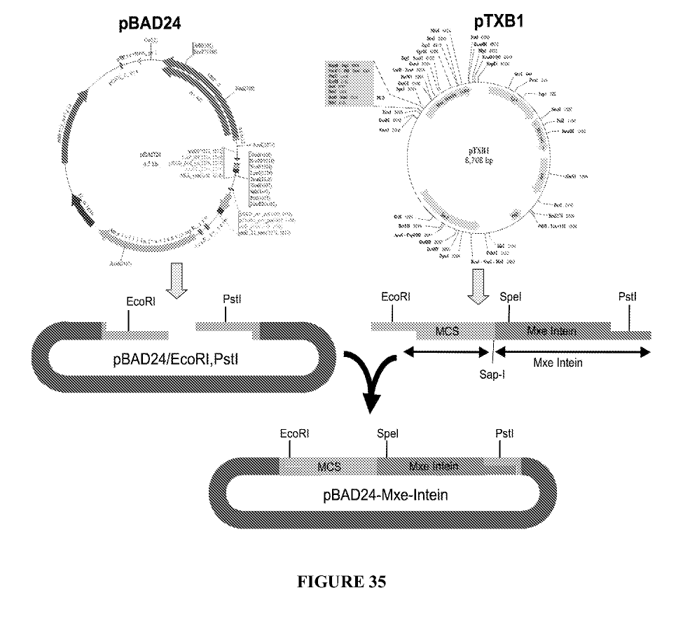

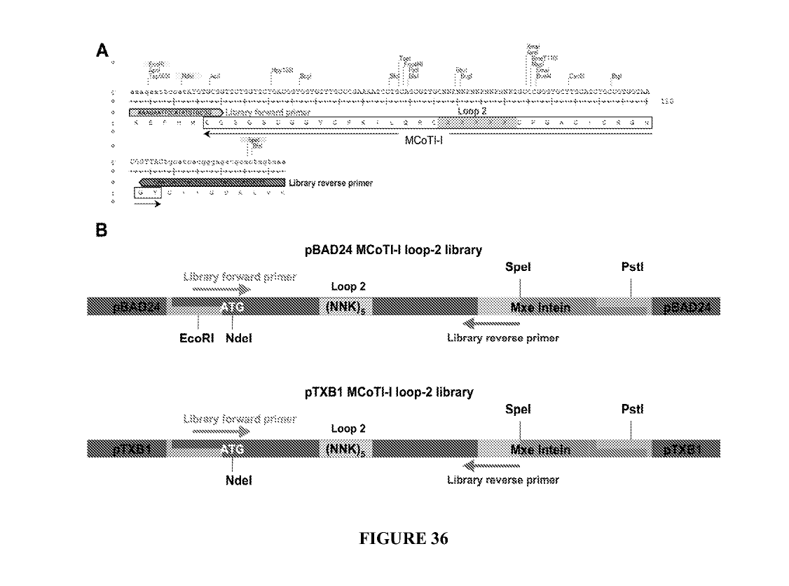

This example shows the cloning, expression and characterization of a combinatorial library based on loop 2 of cyclotide MCoTI-I. For this purpose Applicant has used two different expression vectors: pTXB1 and pBAD24. The Mxe Gyrase intein was subcloned into pBAD24 following the procedure depicted in FIG. 35. Genetically-encoded cyclotide-based libraries were generated at the DNA level using double stranded (ds) DNA inserts with degenerate sequences for the loop 2 of cyclotide MCoTI-I in combination with PCR. Briefly, a long degenerate synthetic oligonucleotide (which encodes the whole cyclotide, ≈100 nt long and the degenerate loop) template is PCR amplified using 5′- and 3′-primers corresponding to the non degenerate flanking regions (FIG. 36A). The degenerate synthetic oligonucleotide template will be synthesized using a NN(G/T) codon scheme for the randomized loops. This scheme uses 32 codons to encode all 20 amino acids and encodes only 1 stop codon. The theoretical for such library is 20⁵ or 3.2×10⁶ sequences. The resulting double stranded degenerate DNA was double digested and then ligated to a linearized intein-containing expression vector to produce a library of pTXB1- or pBAD24-based plasmids (FIG. 36B). These libraries were transformed into electrocompetent E. coli cells to finally obtain a library of cells (about 10⁶). Characterization of the libraries was carried by picking and sequencing of 50 different clones. The results for the pTXB1-based library indicated a good library with a good diversity and an estimated complexity of 1×10⁶.

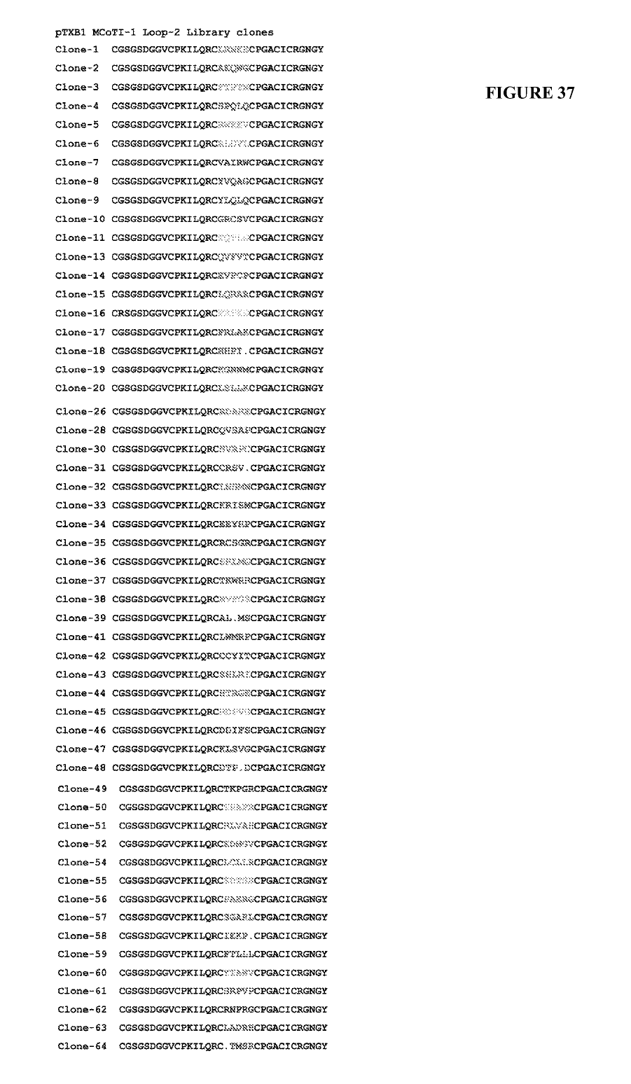

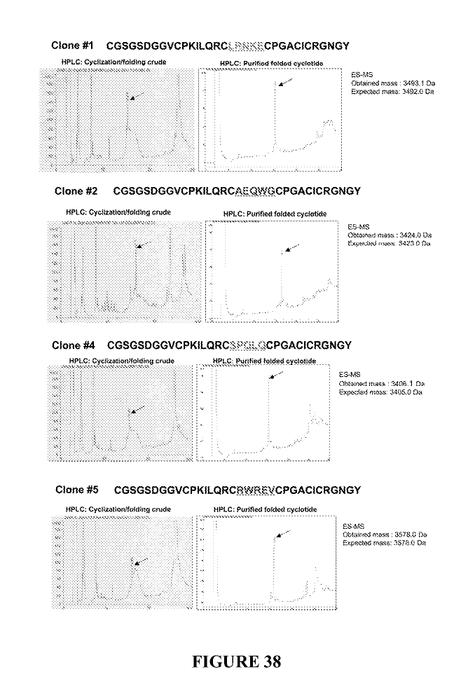

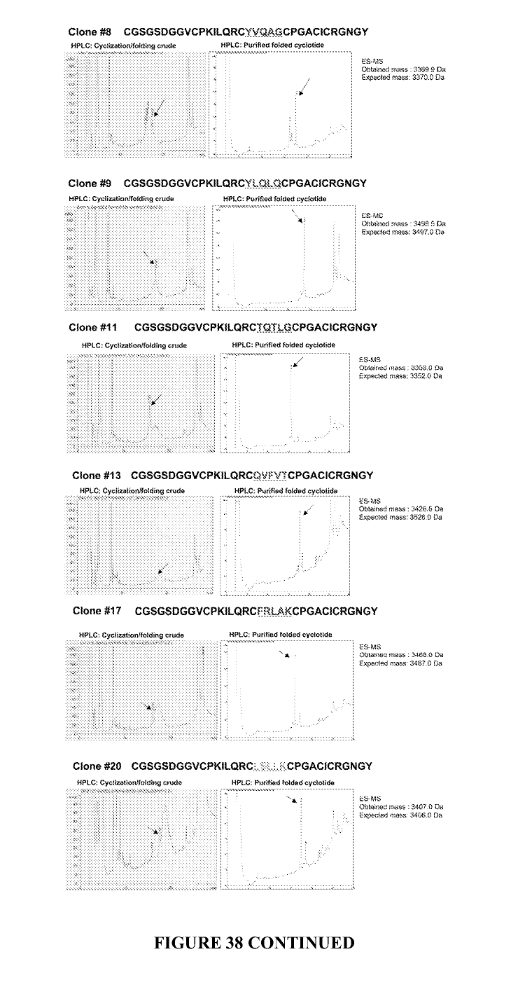

Several clones were individually expressed to check the expression of the corresponding novel cyclotides (FIGS. 37 and 38).

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying or determining if a test agent inhibits formation of a biologically relevant complex in vivo in a cell, wherein the cell comprises:
a template plasmid or vector comprising a first discreet origin of replication operatively linked to a recombinant polynucleotide encoding a N-terminal leader sequence to generate an N-terminal Cys residue, a peptide template to be cyclized and an intein modified to generate a C-terminal thioester in vivo;
a reporter plasmid or vector comprising a second discreet origin of replication operatively linked to one or more interacting domains and a lethality reporter and/or a detectable label;
and where the method comprises culturing the cell under conditions to express the peptide template and subsequently culturing the cell under conditions to express the reporter plasmid or vector; and
selecting cells, thereby determining if a test agent inhibits formation of a biologically relevant complex in vivo in the cell.

2. The method of claim 1, wherein the modified intein comprises a Gyrase or VMA intein, an equivalent or a fragment or each thereof.

3. The method of claim 1, wherein the peptide template to be cyclized further comprises a discrete protein binding domain for isolation of the a template plasmid or vector from the cell.

4. The method of claim 1, wherein the N-terminal leader sequence comprises a leader sequence from the group of methionine, ubiquitin, modified ubiquitin or an equivalent of each thereof.

5. The method of claim 1, wherein the lethality domain comprise a recombinant polynucleotide encoding a Barnase polypeptide or a fragment or an equivalent of each thereof.

6. The method of claim 1, wherein the detectable label comprises a fluorescent label.

7. The method of claim 1, wherein the reporter plasmid or vector and/or the template plasmid or vector further comprises a drug resistant gene.

8. The method of claim 1, wherein the reporter plasmid or vector comprises a first plasmid or vector comprising a fragment of the detectable label fused at the C-terminus or N-terminus of a fragment of one of two the interacting domains and a second plasmid or vector comprising a second fragment of the detectable label fused to the N-terminus or C-terminus of the other interacting domain, wherein the first and the second fragment of the detectable label emit a detectable signal when brought into proximity with each other by the binding or fusion of the interacting domains.

9. The method of claim 1, wherein the reporter plasmid or vector comprises a polycistronic vector.

10. The method of claim 1, wherein the reporter plasmid or vector further comprises a polynucleotide encoding a peptide linker between the interacting domain and the detectable label and/or the lethality reporter.

11. The method of claim 1, wherein the reporter plasmid or vector comprises a first plasmid or vector comprising a first fragment of the lethality reporter fused at the C-terminus or N-terminus of one of the two interacting domains and a second plasmid or vector comprising a second fragment of the lethality reporter fused at the C-terminus or N-terminus of the second interacting domain, and wherein the first and second fragment of the lethality reporter will kill the host cell when brought into proximity with each other by the binding or fusing of the first and second interacting domains.

12. The method of claim 11, wherein the reporter plasmid or vector comprises a polycistronic vector.

13. The method of claim 11 or 12, wherein the reporter plasmid or vector further comprises a polynucleotide encoding a peptide linker between the interacting domain and the lethality reporter.

14. The method of claim 1, wherein the template plasmid or vector and/or the reporter plasmid or vector further comprises one or more of a discrete promoter or a discrete marker.

15. The method of claim 10, wherein the interacting domain comprises a VMA-N-intein or a fragment or an equivalent of each thereof.

16. The method of claim 15, wherein the VMA-N-intein comprises amino acids 1 to 184 of the intein.

17. The method of claim 10, wherein the interacting domain comprises a VMA-C intein or a fragment or an equivalent of each thereof.

18. The method of claim 17, wherein the VMA-C intein unit comprises amino acids 390 to 454 of the VMA-C intein.

19. The method of claim 16, wherein the VMA-N intein and/or VMA-C intein further a peptide of the group: a transactivation domain of p53; a adenylate cyclase domain of EF or a CaM protein that binds the adenylate cyclase domain of EF or a fragment or an equivalent of each thereof.

20. The method of claim 1, wherein the cell is a prokaryotic or a eukaryotic cell.

21. The method of claim 1, wherein the cell is an E. coli cell.

22. The method of claim 1, wherein the cells are selected by selecting cells that survive or remain viable and/or express the detectable label.

23. The method of claim 1, wherein the cells are selected by selecting cells that do not survive or remain viable and/or do not express the detectable label.

24. The method of claim 1, further comprising isolating and sequencing the peptide template from the cell.

25. A template plasmid or vector comprising a first discreet origin of replication operatively linked to a recombinant polynucleotide encoding a N-terminal leader sequence to generate an N-terminal Cys residue in vivo, a peptide template to be cyclized and an intein modified to generate a C-terminal thioester in vivo.

26. A reporter plasmid or vector comprising a discreet origin of replication operatively linked to an interacting domain and a lethality reporter and/or a detectable label.

27. The template plasmid or vector of claim 25, wherein the modified intein comprises a cyclotide or a fragment thereof of the group of Gyrase or VMA intein or a fragment or an equivalent of each thereof.

28. The template plasmid or vector of claim 25, wherein the peptide template to be cyclized further comprises a discrete protein binding domain for isolation of a template plasmid or vector from the cell.

29. The template plasmid or vector of claim 25, wherein the N-terminal leader sequence comprises methionine, ubiquitin, modified ubiquitin or an equivalent of each thereof.

30. The reporter plasmid or vector of claim 26, wherein the lethality reporter comprises a recombinant polynucleotide encoding a Barnase polypeptide or a fragment or an equivalent of each thereof.

31. The reporter plasmid or vector of claim 26 or 30, wherein the detectable label comprises a fluorescent label.

32. The template or reporter plasmid or vector of claim 25, further comprising a drug resistant gene.

33. The reporter plasmid or vector of claim 26, wherein the reporter plasmid or vector comprises a first plasmid or vector comprising a fragment of the detectable label fused at the C-terminus or N-terminus of one of the interacting domain and a second plasmid or vector comprising a second fragment of the detectable label fused to the N-terminus or C-terminus of the other interacting domain, wherein the first and the second fragment of the detectable label emit a detectable signal when brought into proximity with each other by the binding or fusion of the interacting domains.

34. The reporter plasmid or vector of claim 33, wherein the vector or vector comprises a polycistronic vector.

35. The reporter plasmid or vector of claim 26, wherein the reporter plasmid or vector further comprises a polynucleotide encoding a peptide linker between the interacting domain and the detectable label.

36. The reporter plasmid or vector of claim 26, wherein the reporter plasmid or vector comprises a first plasmid or vector comprising a first fragment of the lethality reporter fused at the C-terminus or N-terminus of a first fragment of the interacting domain and a second plasmid or vector comprising a second fragment of the lethality reporter fused at the C-terminus of the other interacting domain, and wherein the first and second fragment of the lethality reporter will kill the host cell when brought into proximity with each other by the binding or fusing of the interacting domains.

37. The reporter plasmid or vector of claim 36, wherein the reporter plasmid or vector comprises a polycistronic vector.

38. The reporter plasmid or vector of claim 26, wherein the reporter plasmid or vector further comprises a polynucleotide encoding a peptide linker between the interacting domain and the detectable label.

39. The template plasmid or vector and/or the reporter plasmid or vector of claim 25, further comprising one or more of a discrete promoter or a discrete marker.

40. The template plasmid or vector of claim 25, wherein the interacting domain comprises a VMA-N-intein or a fragment or an equivalent of each thereof.

41. The template plasmid or vector of claim 40, wherein the VMA-N-intein comprises amino acids 1 to 184 of the intein.

42. The template plasmid or vector of claim 40, wherein the interacting domain comprises a VMA-C intein or a fragment or an equivalent or each thereof.

43. The template plasmid or vector of claim 42, wherein the VMA-C intein unit comprises amino acids 390 to 454 of the VMA-C intein.

44. The template plasmid or vector of claim 40, wherein the VMA-N intein and/or VMA-C intein further a peptide of the group: a transactivation domain of p53; a adenylate cyclase domain of EF or a CaM protein that binds the adenylase cyclase domain of EF or a fragment or an equivalent of each thereof.

45. An isolated host cell comprising one or more of the plasmid or vector of claim 25.

46. The isolated host cell of claim 45, wherein the host cell is a eukaryotic cell or a prokaryotic cell.

47. The isolated host cell of claim 45, wherein the cell is an E. coli cell.

48. An isolated host cell comprising the reporter plasmid or vector of claim 26 and a small molecule.

49. A kit for determining identifying or determining if a test agent inhibits formation of a biologically relevant complex in vivo in a cell comprising the recombinant plasmid or vector of claim 25 and instructions for use.
